(19) United States
(12) Patent Application Publication
(10) Pub. No.: US 2021/0106651 A1
(43) Pub. Date: Apr. 15, 2021

(54) BIO-MIMETIC FORMULATION

(71) Applicant: CyPhi LLC, Miami, FL (US)

(72) Inventors: Abhinav GUATAM, Miami Beach, FL (US); Sean CHENG, Boston, MA (US)

(21) Appl. No.: 17/130,152

(22) Filed: Dec. 22, 2020

Related U.S. Application Data
(62) Division of application No. 16/254,337, filed on Jan. 22, 2019, now abandoned.
(60) Provisional application No. 62/695,238, filed on Jul. 9, 2018.
(60) Provisional application No. 62/619,959, filed on Jan. 22, 2018.

(51) Int. Cl. A61K 38/17; A61P 17/02; A61K 38/21; A61K 38/18; A61K 38/20; A61K 38/19; C07K 14/47

(52) U.S. Cl. CPC A61K 38/1709; A61K 45/06; A61K 38/217; A61K 38/215; A61K 38/1841; A61K 38/1808; A61K 38/2026; A61K 38/204; A61K 38/2053; A61K 38/2066; A61K 38/193; A61K 38/2006; A61K 38/2033; A61K 38/2046; A61K 38/185; A61K 38/1866; C07K 14/47; A61P 17/02

(57) ABSTRACT

The present application provides bio-mimetic formulations, including formulations that mimic mammalian amniotic membrane and/or fluid, but with optimized amounts and formulas of various recombinant peptides. These formulations are stable for extended periods and can be used in various treatment methods including wound healing.

### CROSS-REFERENCE TO RELATED APPLICATIONS

The present application is a Divisional of U.S. application Ser. No. 16/254,337, filed Jan. 22, 2019, which claims the benefit of U.S. Provisional Patent Application Nos. 62/619,959, filed Jan. 22, 2018, and 62/695,238, filed Jul. 9, 2018, the disclosures of each of which are incorporated by reference herein in their entireties.

### FIELD OF THE INVENTION

The present application provides bio-mimetic formulations, including formulations that mimic mammalian amniotic membrane and/or fluid, but with optimized amounts and formulas of various recombinant peptides. These formulations are stable for extended periods and can be used in various treatment methods including wound healing.

### BACKGROUND OF THE INVENTION

The mammalian amniotic membrane is derived from the inner layer of the placenta and is composed of conjoined amnion and chorion membranes. (See, e.g., Koob et al., “Properties of Dehydrated Human and Amnion/Chorion Composite Grafts: Implications for Wound Repair and Soft Tissue Regeneration,” Journal of Biomedical Materials Research B: Applied Biomaterials 00B:000-000 (2014)). The amniotic membrane holds the developing fetus and amniotic fluid, so this thin membrane must possess the structural integrity to support the pregnancy through term. It is a metabolically active tissue which continually remodels the extracellular matrix (ECM) through processes governed by paracrine growth factors. Required nutrients are supplied to the amniotic membranes directly by diffusion out of the amniotic fluid or from the underlying decidua. In addition to physically encasing the amniotic fluid and developing fetus, amniotic membranes play an integral biological role in fetal development and progression of pregnancy; therefore, amniotic membrane grafts harbor significant biological activity, including a number of developmental cytokines. Id.

Storage and preservation is crucial for the success of many applications involving biological materials. To date, a variety of devices and methods have been used to prepare biological materials in a dehydrated form in order to confer benefits such as reduced weight and reduced storage space, and also increased chemical and/or structural stability. While each of these devices have certain benefits, they also come with certain detriments including one or more of the following, prolonged drying time, limited capacity, uneven drying, use of chemicals which can modify the biological materials and compromise their functions. Other methods use sugars to stabilize biological materials prior to freeze-drying. These types of processes, however, can produce ice crystals and damage biological material structures.

What is needed is a bio-mimetic formulation that provides the medical benefits of amniotic fluid and the amniotic membrane, in a more accessible, easier to control embodiment.

### BRIEF SUMMARY OF THE INVENTION

The present invention fulfills these needs by obviated the logistic and cost associated with human tissue recovery and processing. Additionally, the formulations described herein can be normalized to contain precise, repeatable concentrations of any of the components.

In embodiments, provided herein are bio-mimetic formulations, comprising a carrier and any of the following recombinant peptides, including at least 20 of the following recombinant peptides.
Basic fibroblast growth factor (bFGF) at about 4440 (pg/mL) to about 44400 (pg/mL);
Epidermal growth factor (EGF) at about 16.4 (pg/mL) to about 164 (pg/mL);
Granulocyte colony-stimulating factor (GCSF) at about 144 (pg/mL) to about 1440 (pg/mL);
Platelet-derived growth factor (PDGF-AA) at about 34327 (pg/mL) to about 343270 (pg/mL);
Platelet-derived growth factor (PDGF-BB) at about 106 (pg/mL) to about 1060 (pg/mL);
Placental growth factor (PLGF) at about 370 (pg/mL) to about 3700 (pg/mL);
Transforming growth factor alpha (TGF-alpha) at about 3.4 (pg/mL) to about 34 (pg/mL);
Transforming growth factor beta 1 (TGF-B1) at about 1180 (pg/mL) to about 11800 (pg/mL);
interleukin 4 (IL-4) at about 2.2 (pg/mL) to about 22 (pg/mL);
interleukin 6 (IL-6) at about 74 (pg/mL) to about 740 (pg/mL);
interleukin 8 (IL-8) at about 2875 (pg/mL) to about 28750 (pg/mL);
interleukin 10 (IL-10) at about 4.1 (pg/mL) to about 41 (pg/mL);
Tissue inhibitor of metalloproteinase (TIMP-1) at about 16630 (pg/mL) to about 166300 (pg/mL);
Tissue inhibitor of metalloproteinase (TIMP-2) at about 2960 (pg/mL) to about 29600 (pg/mL);
Tissue inhibitor of metalloproteinase (TIMP-4) at about 111 (pg/mL) to about 1110 (pg/mL);
Growth Differentiation Factor (GDF-15) at about 81.25 (pg/mL) to about 812.5 (pg/mL);
Granulocyte macrophage colony-stimulating factor (GM-CSF) at about 0.21 (pg/mL) to about 2.1 (pg/mL);
Interferon gamma (IFNγ) at about 2.75 (pg/mL) to about 27.5 (pg/mL);
Interleukin 1 alpha (IL1-alpha) at about 12.5 (pg/mL) to about 125 (pg/mL);
Interleukin 1 Beta (IFN-β) at about 27.8 (pg/mL) to about 278 (pg/mL);
Interleukin 1 receptor antagonist (IL-1ra) at about 78.5 (pg/mL) to about 785 (pg/mL);
Interleukin 5 (IL-5) at about 2.88 (pg/mL) to about 28.8 (pg/mL);
Interleukin 7 (IL-7) at about 1.37 (pg/mL) to about 13.7 (pg/mL);
Interleukin 12 p40 (IL-12p40) at about 9.55 (pg/mL) to about 95.5 (pg/mL);
Interleukin 12 p70 (IL-12p70) at about 0.79 (pg/mL) to about 7.9 (pg/mL);
Interleukin 15 (IL-15) at about 1.35 (pg/mL) to about 13.5 (pg/mL);
Interleukin 17 (IL-17) at about 1.1 (pg/mL) to about 11 (pg/mL);
Interleukin 16 (IL-16) at about 34.4 (pg/mL) to about 344 (pg/mL);
Macrophage colony-stimulating factor (MCSF) at about 4.3 (pg/mL) to about 43 (pg/mL);
Osteoprotegerin (OPG) at about 319.69 (pg/mL) to about 3196.9 (pg/mL);
B lymphocyte chemoattractant (CXCL13) (BLC) at about 60 (pg/mL) to about 600 (pg/mL);
Chemokine ligand 1 (CCL1) (1-309) at about 4 (pg/mL) to about 40 (pg/mL);
Eotaxin-2 at about 0.13 (pg/mL) to about 1.3 (pg/mL);
Monocyte chemotactic protein 1 (CCL2) (MCP-1) at about 76.95 (pg/mL) to about 769.5 (pg/mL);
Monokine induced by gamma interferon (CXCL9) (MIG) at about 780 (pg/mL) to about 7800 (pg/mL);
Macrophage inflammatory protein 1 alpha (CCL3) (MIP-1α) at about 13.08 (pg/mL) to about 130.8 (pg/mL);
Macrophage inflammatory protein 1 beta (CCL4) (MIP-1β) at about 6.56 (pg/mL) to about 65.6 (pg/mL);
Macrophage inflammatory protein 1D (MIP-5, CCL15) (MIP-1d) at about 4.3 (pg/mL) to about 43 (pg/mL);
Regulated on activation, normal T cell expressed and secreted (CCL5) (RANTES) at about 203 (pg/mL) to about 2030 (pg/mL);
Brain-derived neurotrophic factor (BDNF) at about 45.03 (pg/mL) to about 450.3 (pg/mL);
Bone morphogenetic protein 5 (BMP-5) at about 90.55 (pg/mL) to about 905.5 (pg/mL);
Endocrine gland-derived vascular endothelial growth factor (EG-VEGF) at about 496.68 (pg/mL) to about 4966.8 (pg/mL);
Fibroblast growth factor 4 (FGF-4) at about 353.37 (pg/mL) to about 3533.7 (pg/mL);
Keratinocyte growth factor (FGF-7) at about 46.7 (pg/mL) to about 467 (pg/mL);
Growth hormone (GH) at about 114.23 (pg/mL) to about 1142.3 (pg/mL);
Insulin-like growth factor (IGF-I) at about 27.65 (pg/mL) to about 276.5 (pg/mL);
Insulin-like growth factor binding protein-1 (IGFBP-1) at about 353.51 (pg/mL) to about 3535.1 (pg/mL);
Insulin-like growth factor binding protein-2 (IGFBP-2) at about 1072.52 (pg/mL) to about 10725.2 (pg/mL);
Insulin-like growth factor binding protein-3 (IGFBP-3) at about 7701.19 (pg/mL) to about 77011.9 (pg/mL);
Insulin-like growth factor binding protein-4 (IGFBP-4) at about 2954.34 (pg/mL) to about 29543.4 (pg/mL); and
Insulin-like growth factor binding protein-6 (IGFBP-6) at about 5162.16 (pg/mL) to about 51621.6 (pg/mL).

In embodiments, the bio-mimetic formulations comprise any of the recited recombinant peptides, and in embodiments at least 30 of the recited recombinant peptides.

In further embodiments, the bio-mimetic formulations can further comprise one or more of the following additional agents: Mānuka honey, cannabidiol (CBD—a non-narcotic fraction of Cannabis sativa), or psilocybin (i.e., prodrug derived from mushrooms).

In additional embodiments, the bio-mimetic formulations can consisting essentially of any of the recombinant peptides, including formulations that consist essentially of 20-50 or 30-40 of the recombinant peptides, or can include or consist of each of the recombinant peptides.

In embodiments, the carrier is a lyophilized or dried carrier. In other embodiments, the carrier is a liquid carrier, which can include one or more of a buffer, a salt, water and serum. The liquid carrier can also be an emulsion.

In additional embodiments, the bio-mimetic formulations can include amounts of the recombinant proteins within the ranges recited herein.

In still further embodiments, the bio-mimetic formulations can consist of a carrier and any of the recited recombinant peptides, in additional embodiments 20-45 of the recited recombinant peptides, but without a specified amount of the recombinant peptides.

In yet further embodiments, the bio-mimetic formulations can comprise a carrier and the following recombinant peptides:
at least one of:
   Basic fibroblast growth factor (bFGF) at about 4440 (pg/mL) to about 44400 (pg/mL); or
   Epidermal growth factor (EGF) at about 16.4 (pg/mL) to about 164 (pg/mL); or
   Granulocyte colony-stimulating factor (GCSF) at about 144 (pg/mL) to about 1440 (pg/mL); and
at least one of:
   Platelet-derived growth factor (PDGF-AA) at about 34327 (pg/mL) to about 343270 (pg/mL); or
   Platelet-derived growth factor (PDGF-BB) at about 106 (pg/mL) to about 1060 (pg/mL); or
   Placental growth factor (PLGF) at about 370 (pg/mL) to about 3700 (pg/mL); and
at least one of:
   Transforming growth factor alpha (TGF)-alpha at about 3.4 (pg/mL) to about 34 (pg/mL); or
   Transforming growth factor beta 1 (TGF-B1) at about 1180 (pg/mL) to about 11800 (pg/mL); and
at least one of:
   interleukin 4 (IL-4) at about 2.2 (pg/mL) to about 22 (pg/mL); or
   interleukin 6 (IL-6) at about 74 (pg/mL) to about 740 (pg/mL); or
   interleukin 8 (IL-8) at about 2875 (pg/mL) to about 28750 (pg/mL); or
   interleukin 10 (IL-10) at about 4.1 (pg/mL) to about 41 (pg/mL); and
at least one of:
   Tissue inhibitor of metalloproteinase (TIMP-1) at about 16630 (pg/mL) to about 166300 (pg/mL); or
   Tissue inhibitor of metalloproteinase (TIMP-2) at about 2960 (pg/mL) to about 29600 (pg/mL); or
   Tissue inhibitor of metalloproteinase (TIMP-4) at about 111 (pg/mL) to about 1110 (pg/mL); and
at least one of:
   Growth Differentiation Factor (GDF-15) at about 81.25 (pg/mL) to about 812.5 (pg/mL); or
   Granulocyte macrophage colony-stimulating factor (GM-CSF) at about 0.21 (pg/mL) to about 2.1 (pg/mL); and
Interferon gamma (IFNγ) at about 2.75 (pg/mL) to about 27.5 (pg/mL); and
at least one of:
   Interleukin 1 alpha (IL1-alpha) at about 12.5 (pg/mL) to about 125 (pg/mL); or
   Interleukin 1 Beta (IFN-β) at about 27.8 (pg/mL) to about 278 (pg/mL); or
   Interleukin 1 receptor antagonist (IL-1ra) at about 78.5 (pg/mL) to about 785 (pg/mL); or
   Interleukin 5 (IL-5) at about 2.88 (pg/mL) to about 28.8 (pg/mL); or
   Interleukin 7 (IL-7) at about 1.37 (pg/mL) to about 13.7 (pg/mL); or
   Interleukin 12 p40 (IL-12p40) at about 9.55 (pg/mL) to about 95.5 (pg/mL); or
   Interleukin 12 p70 (IL-12p70) at about 0.79 (pg/mL) to about 7.9 (pg/mL); or
   Interleukin 15 (IL-15) at about 1.35 (pg/mL) to about 13.5 (pg/mL); or
   Interleukin 17 (IL-17) at about 1.1 (pg/mL) to about 11 (pg/mL); or
   Interleukin 16 (IL-16) at about 34.4 (pg/mL) to about 344 (pg/mL); and
Macrophage colony-stimulating factor (MCSF) at about 4.3 (pg/mL) to about 43 (pg/mL); and
Osteoprotegerin (OPG) at about 319.69 (pg/mL) to about 3196.9 (pg/mL); and
B lymphocyte chemoattractant (CXCL13) (BLC) at about 60 (pg/mL) to about 600 (pg/mL); and
Chemokine ligand 1 (CCL1) (1-309) at about 4 (pg/mL) to about 40 (pg/mL); and
Eotaxin-2 at about 0.13 (pg/mL) to about 1.3 (pg/mL); and
Monocyte chemotactic protein 1 (CCL2) (MCP-1) at about 76.95 (pg/mL) to about 769.5 (pg/mL); and
Monokine induced by gamma interferon (CXCL9) (MIG) at about 780 (pg/mL) to about 7800 (pg/mL); and
at least one of:
   Macrophage inflammatory protein 1 alpha (CCL3) (MIP-1α) at about 13.08 (pg/mL) to about 130.8 (pg/mL); or
   Macrophage inflammatory protein 1 beta (CCL4) (MIP-1β) at about 6.56 (pg/mL) to about 65.6 (pg/mL); or
   Macrophage inflammatory protein 1D (MIP-5, CCL15) (MIP-1d) at about 4.3 (pg/mL) to about 43 (pg/mL); and
Regulated on activation, normal T cell expressed and secreted (CCL5) (RANTES) at about 203 (pg/mL) to about 2030 (pg/mL); and
Brain-derived neurotrophic factor (BDNF) at about 45.03 (pg/mL) to about 450.3 (pg/mL); and
Bone morphogenetic protein 5 (BMP-5) at about 90.55 (pg/mL) to about 905.5 (pg/mL); and
Endocrine gland-derived vascular endothelial growth factor (EG-VEGF) at about 496.68 (pg/mL) to about 4966.8 (pg/mL); and
Fibroblast growth factor 4 (FGF-4) at about 353.37 (pg/mL) to about 3533.7 (pg/mL); and
Keratinocyte growth factor (FGF-7) at about 46.7 (pg/mL) to about 467 (pg/mL); and
Growth hormone (GH) at about 114.23 (pg/mL) to about 1142.3 (pg/mL); and
at least one of:
   Insulin-like growth factor (IGF-I) at about 27.65 (pg/mL) to about 276.5 (pg/mL); or
   Insulin-like growth factor binding protein-1 (IGFBP-1) at about 353.51 (pg/mL) to about 3535.1 (pg/mL); or
   Insulin-like growth factor binding protein-2 (IGFBP-2) at about 1072.52 (pg/mL) to about 10725.2 (pg/mL); or
   Insulin-like growth factor binding protein-3 (IGFBP-3) at about 7701.19 (pg/mL) to about 77011.9 (pg/mL); or
   Insulin-like growth factor binding protein-4 (IGFBP-4) at about 2954.34 (pg/mL) to about 29543.4 (pg/mL); or
   Insulin-like growth factor binding protein-6 (IGFBP-6) at about 5162.16 (pg/mL) to about 51621.6 (pg/mL).

Additional embodiments include bio-mimetic formulations that include amounts of the recombinant peptides within these recited ranges.

Also provided herein are methods of preparing the bio-mimetic formulations, as well as methods of treatment, comprising administering the bio-mimetic formulations to a mammalian patient.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

In embodiments, provided herein are various bio-mimetic formulations that include a carrier and various combinations of any of the following recombinant peptides:

TABLE 1
Recombinant Peptides

Basic fibroblast growth factor (bFGF);
Epidermal growth factor (EGF);
Granulocyte colony-stimulating factor (GCSF);
Platelet-derived growth factor (PDGF-AA);
Platelet-derived growth factor (PDGF-BB);
Placental growth factor (PLGF);
Transforming growth factor alpha (TGF - alpha);
Transforming growth factor beta 1 (TGF-B1);
interleukin 4 (IL-4);
interleukin 6 (IL-6);
interleukin 8 (IL-8);
interleukin 10 (IL-10);
Tissue inhibitor of metalloproteinase (TIMP-1);
Tissue inhibitor of metalloproteinase (TIMP-2);
Tissue inhibitor of metalloproteinase (TIMP-4);
Growth Differentiation Factor (GDF-15);
Granulocyte macrophage colony-stimulating factor (GM-CSF);
Interferon gamma (IFNγ);
Interleukin 1 alpha (IL1-alpha);
Interleukin 1 Beta (IFN-β);
Interleukin 1 receptor antagonist (IL-1ra);
Interleukin 5 (IL-5);
Interleukin 7 (IL-7);
Interleukin 12 p40 (IL-12p40);
Interleukin 12 p70 (IL-12p70);
Interleukin 15 (IL-15);
Interleukin 17 (IL-17);
Interleukin 16 (IL-16);
Macrophage colony- stimulating factor (MCSF);
Osteoprotegerin (OPG);
B lymphocyte chemoattractant (CXCL13) (BLC);
Chemokine ligand 1 (CCL1) (I-309);
Eotaxin-2;
Monocyte chemotactic protein 1 (CCL2) (MCP-1);
Monokine induced by gamma interferon (CXCL9) (MIG);
Macrophage inflammatory protein 1 alpha (CCL3) (MIP-1α);
Macrophage inflammatory protein 1 beta (CCL4) (MIP-1β);
Macrophage inflammatory protein 1D (MIP-5, CCL15) (MIP-1d);
Regulated on activation, normal T cell expressed and secreted (CCL5)
(RANTES);
Brain-derived neurotrophic factor (BDNF);
Bone morphogenetic protein 5 (BMP-5);
Endocrine gland- derived vascular endothelial growth factor (EG-VEGF);
Fibroblast growth factor 4 (FGF-4);
Keratinocyte growth factor (FGF-7);
Growth hormone (GH);
Insulin-like growth factor (IGF-I);
Insulin-like growth factor binding protein - 1 (IGFBP-1);
Insulin-like growth factor binding protein - 2 (IGFBP-2);
Insulin-like growth factor binding protein - 3 (IGFBP-3);
Insulin-like growth factor binding protein - 4 (IGFBP-4);
Insulin-like growth factor binding protein - 6 (IGFBP-6)

As used herein, the term “bio-mimetic formulation” refers to a composition that includes components designed to mimic or imitate a biological structure or solution, including for example, the amniotic fluid and/or the amniotic membrane.

The bio-mimetic formulations described herein include a carrier. As used herein, “carrier” means a non-active element(s), ingredient(s) or excipient(s) of the formulations, and can include a liquid, a foam, an emulsion (oil-in-water or water-in-oil), a solid, a gel, a lotion, a semi-solid, and other suitable forms. In embodiments, the carrier of the formulations is a liquid carrier. That is, the carrier has the form of a flowable substance, and suitably includes components such as an aqueous primary component (e.g., water), along with other components dissolved within the primary component, including various pharmaceutically acceptable excipients. Examples of components that can be dissolved within the primary component, include for example, various salts, buffers, diluents, electrolytes, bulking agents, etc. Exemplary excipients which can be used in the carriers include, but are not limited to, a diluent, vehicle, preservative, binder or stabilizing agent. Examples of excipients include, but are not limited to, proteins (e.g., serum albumin), amino acids (e.g., aspartic acid, glutamic acid, lysine, arginine, glycine and histidine), surfactants (e.g., SDS, polysorbate and nonionic surfactant), saccharides (e.g., glucose, sucrose, maltose and trehalas), polyols (e.g., mannitol and sorbitol), fatty acids and phospholipids (e.g., alkyl sulfonates and caprylate). For additional information regarding excipients, see Remington's Pharmaceutical Sciences (by Joseph P. Remington, 18th ed., Mack Publishing Co., Easton, Pa.), which is incorporated herein in its entirety.

In embodiments, the liquid carrier includes serum, which refers to the fluid remaining after blood as coagulated. In additional embodiments, a plasma-like solution can be used as the liquid carrier. For example, a solution such as PLASMA-LYTE A Injection pH 7.4 (Multiple Electrolytes Injection, Type 1, USP) which is a sterile, nonpyrogenic isotonic solution in a single dose container for intravenous administration can be utilized. In embodiments, 100 mL contains 526 mg of Sodium Chloride, USP (NaCl); 502 mg of Sodium Gluconate (C6H11NaO7); 368 mg of Sodium Acetate Trihydrate, USP (C₂H₃NaO₂.3H₂O); 37 mg of Potassium Chloride, USP (KCl); and 30 mg of Magnesium Chloride, USP (MgCl₂.6H₂O). Suitably, it contains no antimicrobial agents. The pH is adjusted with sodium hydroxide to about 7.4 (6.5 to 8.0). One liter has an ionic concentration of 140 mEq sodium, 5 mEq potassium, 3 mEq magnesium, 98 mEq chloride, 27 mEq acetate, and 23 mEq gluconate. The osmolarity is 294 mOsmol/L (calc). Normal physiologic osmolarity range is 280 to 310 mOsmol/L. The caloric content is 21 lcal/L.

Solid carriers can also be used in the bio-mimetic formulations described herein, and can take the forms of various pills, capsules, suppositories, creams, lotions, depot forms and any successor forms, etc. In addition, a solid carrier can also include a bio-mimetic formulation which was prepared in a liquid form, and then the liquid removed via dehydration, lyophilization, evaporation, or the formulation freeze dried, or otherwise prepared, as a solid form.

The terms “recombinant polypeptide,” “recombinant peptide,” “recombinant protein,” and “recombinant protein fragment” are used interchangeably herein to refer to a polymer of amino acid residues that is synthesized synthetically (i.e., not simply isolated from a biological sample), suitably produced by expression of a recombinant (synthetic) nucleic acid. The terms apply to amino acid polymers in which one or more amino acid residue is an artificial chemical mimetic of a corresponding naturally occurring amino acid, as well as to naturally occurring amino acid polymers and non-naturally occurring amino acid polymers.

Methods of preparing recombinant peptides are well known in the art, and include various vector and protein expression systems, including viral vectors, plasmids, liposome based-transfection, etc., and the use of bacterial (e.g., E. Coli) or other host cells for replication and protein production. Exemplary methods can be found for example in Protein Expression Technologies: Current Status and Future Trends, Baneyx ed., Horizon Bioscience, Norfolk, UK (2004), the disclosure of which is incorporated by reference herein in its entirety, including the methods disclosed therein. Methods of preparing recombinant peptides also include various filtration and purification steps, such as column filtration, dialysis, etc.

In embodiments, the bio-mimetic formulations include at least 10 of the recombinant peptides listed in Table 1. In further embodiments, the bio-mimetic formulations include all of the recombinant peptides, or between 1-51 of the recombinant peptides, and in other embodiments at least 1, at least 2, at least 3, at least 4, at least 5, at least 10, at least 15, at least 20, at least 25, at least 30, at least 35, at least 40, at least 45, at least 50, or in some embodiments, the bio-mimetic formulations include each of the 51 recombinant peptides listed in Table 1.

In additional embodiments, the bio-mimetic formulations include all of the recombinant peptides, or between 1-51 of the recombinant peptides, and in other embodiments at least 1, at least 2, at least 3, at least 4, at least 5, at least 10, at least 15, at least 20, at least 25, at least 30, at least 35, at least 40, at least 45, at least 50, or in some embodiments, the bio-mimetic formulations include each of the 51 recombinant peptides of the recombinant peptides listed below, at the amounts set forth in the various Tables herein. The amounts of the recombinant peptides described herein are expressed as picograms of recombinant protein/milliliter of formulation (pg/mL). The volume of the formulation includes the volume of the carrier(s) used to prepare the formulation. In further embodiments, the amounts of the recombinant peptides can be measured in weight/weight, with the weight of the formulation including the weight of the carrier(s) used to prepare the formulation.

TABLE 2
Recombinant Proteins and Exemplary Amounts

Basic fibroblast growth factor (bFGF) at about 4440 (pg/mL) to about 44400
(pg/mL);
Epidermal growth factor (EGF) at about 16.4 (pg/mL) to about 164 (pg/mL);
Granulocyte colony-stimulating factor (GCSF) at about 144 (pg/mL) to about
1440 (pg/mL);
Platelet-derived growth factor (PDGF-AA) at about 34327 (pg/mL) to about
343270 (pg/mL);
Platelet-derived growth factor (PDGF-BB) at about 106 (pg/mL) to about 1060
(pg/mL);
Placental growth factor (PLGF) at about 370 (pg/mL) to about 3700 (pg/mL);
Transforming growth factor alpha (TGF - alpha) at about 3.4 (pg/mL) to about
34 (pg/mL);
Transforming growth factor beta 1 (TGF-B1) at about 1180 (pg/mL) to about
11800 (pg/mL);
interleukin 4 (IL-4) at about 2.2 (pg/mL) to about 22 (pg/mL);
interleukin 6 (IL-6) at about 74 (pg/mL) to about 740 (pg/mL);
interleukin 8 (IL-8) at about 2875 (pg/mL) to about 28750 (pg/mL);
interleukin 10 (IL-10) at about 4.1 (pg/mL) to about 41 (pg/mL);
Tissue inhibitor of metalloproteinase (TIMP-1) at about 16630 (pg/mL) to
about 166300 (pg/mL);
Tissue inhibitor of metalloproteinase (TIMP-2) at about 2960 (pg/mL) to about
29600 (pg/mL);
Tissue inhibitor of metalloproteinase (TIMP-4) at about 111 (pg/mL) to about
1110 (pg/mL);
Growth Differentiation Factor (GDF-15) at about 81.25 (pg/mL) to about 812.5
(pg/mL);
Granulocyte macrophage colony-stimulating factor (GM-CSF) at about 0.21
(pg/mL) to about 2.1 (pg/mL);
Interferon gamma (IFNγ) at about 2.75 (pg/mL) to about 27.5 (pg/mL);
Interleukin 1 alpha (IL1-alpha) at about 12.5 (pg/mL) to about 125 (pg/mL);
Interleukin 1 Beta (IFN-β) at about 27.8 (pg/mL) to about 278 (pg/mL);
Interleukin 1 receptor antagonist (IL-1ra) at about 78.5 (pg/mL) to about 785
(pg/mL);
Interleukin 5 (IL-5) at about 2.88 (pg/mL) to about 28.8 (pg/mL);
Interleukin 7 (IL-7) at about 1.37 (pg/mL) to about 13.7 (pg/mL);
Interleukin 12 p40 (IL-12p40) at about 9.55 (pg/mL) to about 95.5 (pg/mL);
Interleukin 12 p70 (IL-12p70) at about 0.79 (pg/mL) to about 7.9 (pg/mL);
Interleukin 15 (IL-15) at about 1.35 (pg/mL) to about 13.5 (pg/mL);
Interleukin 17 (IL-17) at about 1.1 (pg/mL) to about 11 (pg/mL);
Interleukin 16 (IL-16) at about 34.4 (pg/mL) to about 344(pg/mL);
Macrophage colony- stimulating factor (MCSF) at about 4.3 (pg/mL) to about
43 (pg/mL);
Osteoprotegerin (OPG) at about 319.69 (pg/mL) to about 3196.9 (pg/mL);
B lymphocyte chemoattractant (CXCL13) (BLC) at about 60 (pg/mL) to about
600 (pg/mL);
Chemokine ligand 1 (CCL1) (I-309) at about 4 (pg/mL) to about 40 (pg/mL);
Eotaxin-2 at about 0.13 (pg/mL) to about 1.3 (pg/mL);
Monocyte chemotactic protein 1 (CCL2) (MCP-1) at about 76.95 (pg/mL) to
about 769.5 (pg/mL);
Monokine induced by gamma interferon (CXCL9) (MIG) at about 780 (pg/mL)
to about 7800 (pg/mL);
Macrophage inflammatory protein 1 alpha (CCL3) (MIP-1α) at about 13.08
(pg/mL) to about 130.8 (pg/mL);
Macrophage inflammatory protein 1 beta (CCL4) (MIP-1β) at about 6.56
(pg/mL) to about 65.6 (pg/mL);
Macrophage inflammatory protein ID (MIP-5, CCL15) (MIP-1d) at about 4.3
(pg/mL) to about 43 (pg/mL);
Regulated on activation, normal T cell expressed and secreted (CCL5)
(RANTES) at about 203 (pg/mL) to about 2030 (pg/mL);
Brain-derived neurotrophic factor (BDNF) at about 45.03 (pg/mL) to about
450.3 (pg/mL);
Bone morphogenetic protein 5 (BMP-5) at about 90.55 (pg/mL) to about 905.5
(pg/mL);
Endocrine gland- derived vascular endothelial growth factor (EG-VEGF) at
about 496.68 (pg/mL) to about 4966.8 (pg/mL);
Fibroblast growth factor 4 (FGF-4) at about 353.37 (pg/mL) to about 3533.7
(pg/mL);
Keratinocyte growth factor (FGF-7) at about 46.7 (pg/mL) to about 467
(pg/mL);
Growth hormone (GH) at about 114.23 (pg/mL) to about 1142.3 (pg/mL);
Insulin-like growth factor (IGF-I) at about 27.65 (pg/mL) to about 276.5
(pg/mL);
Insulin-like growth factor binding protein - 1 (IGFBP-1) at about 353.51
(pg/mL) to about 3535.1 (pg/mL);
Insulin-like growth factor binding protein - 2 (IGFBP-2) at about 1072.52
(pg/mL) to about 10725.2 (pg/mL);
Insulin-like growth factor binding protein - 3 (IGFBP-3) at about 7701.19
(pg/mL) to about 77011.9 (pg/mL);
Insulin-like growth factor binding protein - 4 (IGFBP-4) at about 2954.34
(pg/mL) to about 29543.4 (pg/mL); and
Insulin-like growth factor binding protein - 6 (IGFBP-6) at about 5162.16
(pg/mL) to about 51621.6 (pg/mL).

As described herein, it has been surprisingly found that the amounts (pg/mL) of the recombinant peptides included in the bio-mimetic formulations, which are approximately three or more times above those found naturally in the amniotic membrane or amniotic fluid, can be prepared together and provided to a mammalian patient for the treatment of various conditions, ailments, diseases, etc.

In further embodiments, the bio-mimetic formulations described herein include one or more additional proteins or other active agents. Exemplary proteins and other active agents include, but are not limited to, Manuka honey, cannabidiol (CBD—a non-narcotic fraction of Cannabis sativa), psilocybin (i.e., prodrug derived from mushrooms), etc.

In additional embodiments, the bio-mimetic formulations can consist essentially of 1-51, or in other embodiments 5-51, or 10-51 of the recombinant peptides listed in Table 1, or 10-51 of the recombinant peptides at the amounts specified in the various Tables disclosed herein. In bio-mimetic formulations that consist essentially of a number of recombinant peptides (e.g., 1051, 5-51, 10-51, 20-51, 30-51, 40-51, 10-50, 10-45, 10-40, 10-35, 10-30, 10-25, 10-20, 10-15, 20-40, 20-30, 30-40, or 1, 2, 3, 4, 5, 10, 15, 20, 25, 30, 35, 40, 45, 50, or 51, etc., of the recombinant peptides, including values and ranges within these ranges), the bio-mimetic formulations do not include any additional recombinant peptides from those recited in Table 1, or any additional active peptides or proteins, or other active agents, but the bio-mimetic formulations can include additional non-active ingredients or excipients (including those listed herein), that do not affect the basic and novel characteristics of the formulations.

In still further embodiments, the bio-mimetic formulations can include any of, and in embodiments, at least 10 of or at least 20 of the recombinant peptides described herein at the following amounts:

TABLE 3
Recombinant Proteins and Exemplary Amounts (Lower Range)

Basic fibroblast growth factor (bFGF) at about 13320 (pg/mL) to about 44400
(pg/mL);
Epidermal growth factor (EGF) at about 49.2 (pg/mL) to about 164 (pg/mL);
Granulocyte colony-stimulating factor (GCSF) at about 432 (pg/mL) to about
1440 (pg/mL);
Platelet-derived growth factor (PDGF-AA) at about 102981 (pg/mL) to about
343270 (pg/mL);
Platelet-derived growth factor (PDGF-BB) at about 318 (pg/mL) to about 1060
(pg/mL);
Placental growth factor (PLGF) at about 1110 (pg/mL) to about 3700 (pg/mL);
Transforming growth factor alpha (TGF - alpha) at about 10.2 (pg/mL) to about
34 (pg/mL);
Transforming growth factor beta 1 (TGF-B1) at about 3540 (pg/mL) to about
11800 (pg/mL);
interleukin 4 (IL-4) at about 6.6 (pg/mL) to about 22 (pg/mL);
interleukin 6 (IL-6) at about 222 (pg/mL) to about 740 (pg/mL);
interleukin 8 (IL-8) at about 8625 (pg/mL) to about 28750 (pg/mL);
interleukin 10 (IL-10) at about 12.3 (pg/mL) to about 41 (pg/mL);
Tissue inhibitor of metalloproteinase (TIMP-1) at about 49890 (pg/mL) to
about 166300 (pg/mL);
Tissue inhibitor of metalloproteinase (TIMP-2) at about 8880 (pg/mL) to about
29600 (pg/mL);
Tissue inhibitor of metalloproteinase (TIMP-4) at about 333 (pg/mL) to about
1110 (pg/mL);
Growth Differentiation Factor (GDF-15) at about 243.75 (pg/mL) to about
812.5 (pg/mL);
Granulocyte macrophage colony-stimulating factor (GM-CSF) at about 0.63
(pg/mL) to about 2.1 (pg/mL);
Interferon gamma (IFNγ) at about 8.25 (pg/mL) to about 27.5 (pg/mL);
Interleukin 1 alpha (IL1-alpha) at about 37.5 (pg/mL) to about 125 (pg/mL);
Interleukin 1 Beta (IFN-β) at about 83.4 (pg/mL) to about 278 (pg/mL);
Interleukin 1 receptor antagonist (IL-1ra) at about 235.5 (pg/mL) to about 785
(pg/mL);
Interleukin 5 (IL-5) at about 8.64 (pg/mL) to about 28.8 (pg/mL);
Interleukin 7 (IL-7) at about 4.11 (pg/mL) to about 13.7 (pg/mL);
Interleukin 12 p40 (IL-12p40) at about 28.65 (pg/mL) to about 95.5 (pg/mL);
Interleukin 12 p70 (IL-12p70) at about 2.37 (pg/mL) to about 7.9 (pg/mL);
Interleukin 15 (IL-15) at about 4.05 (pg/mL) to about 13.5 (pg/mL);
Interleukin 17 (IL-17) at about 3.3 (pg/mL) to about 11 (pg/mL);
Interleukin 16 (IL-16) at about 103.2 (pg/mL) to about 344(pg/mL);
Macrophage colony- stimulating factor (MCSF) at about 12.9 (pg/mL) to about
43 (pg/mL);
Osteoprotegerin (OPG) at about 959.07 (pg/mL) to about 3196.9 (pg/mL);
B lymphocyte chemoattractant (CXCL13) (BLC) at about 180 (pg/mL) to about
600 (pg/mL);
Chemokine ligand 1 (CCL1) (I-309) at about 12 (pg/mL) to about 40 (pg/mL);
Eotaxin-2 at about 0.39 (pg/mL) to about 1.3 (pg/mL);
Monocyte chemotactic protein 1 (CCL2) (MCP-1) at about 230.85 (pg/mL) to
about 769.5 (pg/mL);
Monokine induced by gamma interferon (CXCL9) (MIG) at about 2340
(pg/mL) to about 7800 (pg/mL);
Macrophage inflammatory protein 1 alpha (CCL3) (MIP-1α) at about 39.24
(pg/mL) to about 130.8 (pg/mL);
Macrophage inflammatory protein 1 beta (CCL4) (MIP-1β) at about 19.68
(pg/mL) to about 65.6 (pg/mL);
Macrophage inflammatory protein ID (MIP-5, CCL15) (MIP-1d) at about 12.9
(pg/mL) to about 43 (pg/mL);
Regulated on activation, normal T cell expressed and secreted (CCL5)
(RANTES) at about 609 (pg/mL) to about 2030 (pg/mL);
Brain-derived neurotrophic factor (BDNF) at about 135.09 (pg/mL) to about
450.3 (pg/mL);
Bone morphogenetic protein 5 (BMP-5) at about 271.65 (pg/mL) to about 905.5
(pg/mL);
Endocrine gland- derived vascular endothelial growth factor (EG-VEGF) at
about 1490.04 (pg/mL) to about 4966.8 (pg/mL);
Fibroblast growth factor 4 (FGF-4) at about 1060.11 (pg/mL) to about 3533.7
(pg/mL);
Keratinocyte growth factor (FGF-7) at about 140.1 (pg/mL) to about 467
(pg/mL);
Growth hormone (GH) at about 342.69 (pg/mL) to about 1142.3 (pg/mL);
Insulin-like growth factor (IGF-I) at about 82.95 (pg/mL) to about 276.5
(pg/mL);
Insulin-like growth factor binding protein - 1 (IGFBP-1) at about 1060.53
(pg/mL) to about 3535.1 (pg/mL);
Insulin-like growth factor binding protein - 2 (IGFBP-2) at about 3217.56
(pg/mL) to about 10725.2 (pg/mL);
Insulin-like growth factor binding protein - 3 (IGFBP-3) at about 23103.57
(pg/mL) to about 77011.9 (pg/mL);
Insulin-like growth factor binding protein - 4 (IGFBP-4) at about 8863.02
(pg/mL) to about 29543.4 (pg/mL); and
Insulin-like growth factor binding protein - 6 (IGFBP-6) at about 15486.48
(pg/mL) to about 51621.6 (pg/mL).

In still further embodiments, the bio-mimetic formulations can include any of, or in embodiments at least 10 or or at least 20 of the recombinant peptides described herein at the following amounts:

TABLE 4
Recombinant Proteins and Exemplary Amounts (Upper Range)

Basic fibroblast growth factor (bFGF) at about 13320 (pg/mL) to about 44400
(pg/mL);
Epidermal growth factor (EGF) at about 49.2 (pg/mL) to about 164 (pg/mL);
Granulocyte colony-stimulating factor (GCSF) at about 432 (pg/mL) to about
1440 (pg/mL);
Platelet-derived growth factor (PDGF-AA) at about 102981 (pg/mL) to about
343270 (pg/mL);
Platelet-derived growth factor (PDGF-BB) at about 318 (pg/mL) to about 1060
(pg/mL);
Placental growth factor (PLGF) at about 1110 (pg/mL) to about 3700 (pg/mL);
Transforming growth factor alpha (TGF - alpha) at about 10.2 (pg/mL) to about
34 (pg/mL);
Transforming growth factor beta 1 (TGF-B1) at about 3540 (pg/mL) to about
11800 (pg/mL);
interleukin 4 (IL-4) at about 6.6 (pg/mL) to about 22 (pg/mL);
interleukin 6 (IL-6) at about 222 (pg/mL) to about 740 (pg/mL);
interleukin 8 (IL-8) at about 8625 (pg/mL) to about 28750 (pg/mL);
interleukin 10 (IL-10) at about 12.3 (pg/mL) to about 41 (pg/mL);
Tissue inhibitor of metalloproteinase (TIMP-1) at about 49890 (pg/mL) to
about 166300 (pg/mL);
Tissue inhibitor of metalloproteinase (TIMP-2) at about 8880 (pg/mL) to about
29600 (pg/mL);
Tissue inhibitor of metalloproteinase (TIMP-4) at about 333 (pg/mL) to about
1110 (pg/mL);
Growth Differentiation Factor (GDF-15) at about 243.75 (pg/mL) to about
812.5 (pg/mL);
Granulocyte macrophage colony-stimulating factor (GM-CSF) at about 0.63
(pg/mL) to about 2.1 (pg/mL);
Interferon gamma (IFNγ) at about 8.25 (pg/mL) to about 27.5 (pg/mL);
Interleukin 1 alpha (IL1-alpha) at about 37.5 (pg/mL) to about 125 (pg/mL);
Interleukin 1 Beta (IFN-β) at about 83.4 (pg/mL) to about 278 (pg/mL);
Interleukin 1 receptor antagonist (IL-1ra) at about 235.5 (pg/mL) to about 785
(pg/mL);
Interleukin 5 (IL-5) at about 8.64 (pg/mL) to about 28.8 (pg/mL);
Interleukin 7 (IL-7) at about 4.11 (pg/mL) to about 13.7 (pg/mL);
Interleukin 12 p40 (IL-12p40) at about 28.65 (pg/mL) to about 95.5 (pg/mL);
Interleukin 12 p70 (IL-12p70) at about 2.37 (pg/mL) to about 7.9 (pg/mL);
Interleukin 15 (IL-15) at about 4.05 (pg/mL) to about 13.5 (pg/mL);
Interleukin 17 (IL-17) at about 3.3 (pg/mL) to about 11 (pg/mL);
Interleukin 16 (IL-16) at about 103.2 (pg/mL) to about 344(pg/mL);
Macrophage colony- stimulating factor (MCSF) at about 12.9 (pg/mL) to about
43 (pg/mL);
Osteoprotegerin (OPG) at about 959.07 (pg/mL) to about 3196.9 (pg/mL);
B lymphocyte chemoattractant (CXCL13) (BLC) at about 180 (pg/mL) to about
600 (pg/mL);
Chemokine ligand 1 (CCL1) (I-309) at about 12 (pg/mL) to about 40 (pg/mL);
Eotaxin-2 at about 0.39 (pg/mL) to about 1.3 (pg/mL);
Monocyte chemotactic protein 1 (CCL2) (MCP-1) at about 230.85 (pg/mL) to
about 769.5 (pg/mL);
Monokine induced by gamma interferon (CXCL9) (MIG) at about 2340
(pg/mL) to about 7800 (pg/mL);
Macrophage inflammatory protein 1 alpha (CCL3) (MIP-1α) at about 39.24
(pg/mL) to about 130.8 (pg/mL);
Macrophage inflammatory protein 1 beta (CCL4) (MIP-1β) at about 19.68
(pg/mL) to about 65.6 (pg/mL);
Macrophage inflammatory protein ID (MIP-5, CCL15) (MIP-1d) at about 12.9
(pg/mL) to about 43 (pg/mL);
Regulated on activation, normal T cell expressed and secreted (CCL5)
(RANTES) at about 609 (pg/mL) to about 2030 (pg/mL);
Brain-derived neurotrophic factor (BDNF) at about 135.09 (pg/mL) to about
450.3 (pg/mL);
Bone morphogenetic protein 5 (BMP-5) at about 271.65 (pg/mL) to about 905.5
(pg/mL);
Endocrine gland- derived vascular endothelial growth factor (EG-VEGF) at
about 1490.04 (pg/mL) to about 4966.8 (pg/mL);
Fibroblast growth factor 4 (FGF-4) at about 1060.11 (pg/mL) to about 3533.7
(pg/mL);
Keratinocyte growth factor (FGF-7) at about 140.1 (pg/mL) to about 467
(pg/mL);
Growth hormone (GH) at about 342.69 (pg/mL) to about 1142.3 (pg/mL);
Insulin-like growth factor (IGF-I) at about 82.95 (pg/mL) to about 276.5
(pg/mL);
Insulin-like growth factor binding protein - 1 (IGFBP-1) at about 1060.53
(pg/mL) to about 3535.1 (pg/mL);
Insulin-like growth factor binding protein - 2 (IGFBP-2) at about 3217.56
(pg/mL) to about 10725.2 (pg/mL);
Insulin-like growth factor binding protein - 3 (IGFBP-3) at about 23103.57
(pg/mL) to about 77011.9 (pg/mL);
Insulin-like growth factor binding protein - 4 (IGFBP-4) at about 8863.02
(pg/mL) to about 29543.4 (pg/mL); and
Insulin-like growth factor binding protein - 6 (IGFBP-6) at about 15486.48
(pg/mL) to about 51621.6 (pg/mL)

In addition to those picogram/milligram amounts and ranges of the recombinant peptides described herein, the bio-mimetic formulations can also include various different amounts, ranges and ratios of the recited recombinant peptides, including specific amounts within those ranges described herein. In embodiments, the amounts of the recombinant peptides can be optimized for use in various patient populations, for treatment of various conditions or diseases, etc. Modification of the amounts of the recombinant peptides can be achieved by reducing or increasing the amounts of the recombinant peptides (including increasing the amount of certain recombinant peptides while reducing the amount of other recombinant peptides), by various amounts. For example, one or more of the recombinant peptides in the bio-mimetic formulations can be increased or reduced relative to one or more other recombinant peptides in the bio-mimetic formulations by about 1-500%, about 1-200%, about 1-100%, about 1-90%, about 1-80%, about 1-70%, about 1-60%, about 1-50%, about 1-40%, about 1-30%, about 1-20%, about 1-10%, about 1-5%, about 10%-20%, about 20%-30%, about 30%-40%, about 40%-50%, about 50%-60%, about 60%-70%, about 70%-80%, about 80%-90%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, etc. In addition, the amounts of various recombinant peptides can be increased by the amounts noted, while the amounts of other recombinant peptides are reduced.

In further embodiments, the amounts of the various recombinant peptides can be increased or reduced by 0.1-times, 2-times, 5-times, 10-times, 100-times, or even 1000-times above and below those ranges provided herein. For example Table 5 below shows ranges 10-times higher and 10-times lower than those listed above:

TABLE 5
Recombinant Proteins and Exemplary Amounts (10X lower and higher)

Basic fibroblast growth factor (bFGF) at about 444 (pg/mL) to about 444000
(pg/mL);
Epidermal growth factor (EGF) at about 1.64 (pg/mL) to about 1640 (pg/mL);
Granulocyte colony-stimulating factor (GCSF) at about 14.4 (pg/mL) to about
14400 (pg/mL);
Platelet-derived growth factor (PDGF-AA) at about 3432.7 (pg/mL) to about
3432700 (pg/mL);
Platelet-derived growth factor (PDGF-BB) at about 10.6 (pg/mL) to about
10600 (pg/mL);
Placental growth factor (PLGF) at about 37.0 (pg/mL) to about 37000 (pg/mL);
Transforming growth factor alpha (TGF - alpha) at about 0.34 (pg/mL) to about
340 (pg/mL);
Transforming growth factor beta 1 (TGF-B1) at about 118.0 (pg/mL) to about
118000 (pg/mL);
interleukin 4 (IL-4) at about 0.22 (pg/mL) to about 220 (pg/mL);
interleukin 6 (IL-6) at about 7.4 (pg/mL) to about 7400 (pg/mL);
interleukin 8 (IL-8) at about 287.5 (pg/mL) to about 287500 (pg/mL);
interleukin 10 (IL-10) at about 0.41 (pg/mL) to about 410 (pg/mL);
Tissue inhibitor of metalloproteinase (TIMP-1) at about 1663.0 (pg/mL) to
about 1663000 (pg/mL);
Tissue inhibitor of metalloproteinase (TIMP-2) at about 296.0 (pg/mL) to about
296000 (pg/mL);
Tissue inhibitor of metalloproteinase (TIMP-4) at about 11.1 (pg/mL) to about
11100 (pg/mL);
Growth Differentiation Factor (GDF-15) at about 8.125 (pg/mL) to about 8125
(pg/mL);
Granulocyte macrophage colony-stimulating factor (GM-CSF) at about 0.021
(pg/mL) to about 21 (pg/mL);
Interferon gamma (IFNγ) at about 0.275 (pg/mL) to about 275 (pg/mL);
Interleukin 1 alpha (IL1-alpha) at about 1.25 (pg/mL) to about 125. (pg/mL);
Interleukin 1 Beta (IFN-β) at about 2.78 (pg/mL) to about 2780 (pg/mL);
Interleukin 1 receptor antagonist (IL-1ra) at about 7.85 (pg/mL) to about 7850
(pg/mL);
Interleukin 5 (IL-5) at about 0.288 (pg/mL) to about 288 (pg/mL);
Interleukin 7 (IL-7) at about 0.137 (pg/mL) to about 1.37 (pg/mL);
Interleukin 12 p40 (IL-12p40) at about 0.955 (pg/mL) to about 955 (pg/mL);
Interleukin 12 p70 (IL-12p70) at about 0.079 (pg/mL) to about 79 (pg/mL);
Interleukin 15 (IL-15) at about 0.135 (pg/mL) to about 135 (pg/mL);
Interleukin 17 (IL-17) at about 0.11 (pg/mL) to about 110 (pg/mL);
Interleukin 16 (IL-16) at about 3.44 (pg/mL) to about 3440 (pg/mL);
Macrophage colony- stimulating factor (MCSF) at about 0.43 (pg/mL) to about
430 (pg/mL);
Osteoprotegerin (OPG) at about 31.969 (pg/mL) to about 31969 (pg/mL);
B lymphocyte chemoattractant (CXCL13) (BLC) at about 6.0 (pg/mL) to about
6000 (pg/mL);
Chemokine ligand 1 (CCL1) (I-309) at about 0.4 (pg/mL) to about 400
(pg/mL);
Eotaxin-2 at about 0.013 (pg/mL) to about 13 (pg/mL);
Monocyte chemotactic protein 1 (CCL2) (MCP-1) at about 7.695 (pg/mL) to
about 7695 (pg/mL);
Monokine induced by gamma interferon (CXCL9) (MIG) at about 78.0
(pg/mL) to about 78000 (pg/mL);
Macrophage inflammatory protein 1 alpha (CCL3) (MIP-1α) at about 1.308
(pg/mL) to about 1308 (pg/mL);
Macrophage inflammatory protein 1 beta (CCL4) (MIP-Iβ) at about 0.656
(pg/mL) to about 656 (pg/mL);
Macrophage inflammatory protein ID (MIP-5, CCL15) (MIP-1d) at about 0.43
(pg/mL) to about 430 (pg/mL);
Regulated on activation, normal T cell expressed and secreted (CCL5)
(RANTES) at about 20.3 (pg/mL) to about 20300 (pg/mL);
Brain-derived neurotrophic factor (BDNF) at about 4.503 (pg/mL) to about
45030 (pg/mL);
Bone morphogenetic protein 5 (BMP-5) at about 9.055 (pg/mL) to about 9055
(pg/mL);
Endocrine gland- derived vascular endothelial growth factor (EG-VEGF) at
about 49.668 (pg/mL) to about 49668 (pg/mL);
Fibroblast growth factor 4 (FGF-4) at about 35.337 (pg/mL) to about 35337
(pg/mL);
Keratinocyte growth factor (FGF-7) at about 4.67 (pg/mL) to about 4670
(pg/mL);
Growth hormone (GH) at about 11.423 (pg/mL) to about 114230 (pg/mL);
Insulin-like growth factor (IGF-I) at about 2.765 (pg/mL) to about 2765
(pg/mL);
Insulin-like growth factor binding protein - 1 (IGFBP-1) at about 35.351
(pg/mL) to about 35351 (pg/mL);
Insulin-like growth factor binding protein - 2 (IGFBP-2) at about 107.252
(pg/mL) to about 107252 (pg/mL);
Insulin-like growth factor binding protein - 3 (IGFBP-3) at about 770.119
(pg/mL) to about 770119 (pg/mL);
Insulin-like growth factor binding protein - 4 (IGFBP-4) at about 295.434
(pg/mL) to about 295434 (pg/mL); and
Insulin-like growth factor binding protein - 6 (IGFBP-6) at about 516.216
(pg/mL) to about 516216 (pg/mL).

Ratios of the amounts of the various recombinant proteins can be modified from those disclosed herein to provide formulations that have improved or specific biological effects in a target patient or population. For example, the ratio of one or more of the recited growth factors to one or more of the recited interleukins can be modified (increased or decreased) to provide an improve or enhanced effect.

In additional embodiments, the bio-mimetic formulation consists of a carrier and each of the recombinant peptides recited in Table 1, or each of the recombinant peptides recited in Table 2 at the amounts recited, or each of the recombinant peptides recited in Table 3 at the amounts recited, or each of the recombinant peptides recited in Table 4 at the amounts recited. As described herein, preparation of bio-mimetic formulations consisting of each of the recombinant peptides at amounts that are least 2-fold, for example at least 3-fold higher (including about 10-times higher) than those found in nature, provide unexpected benefits to patients. In addition, it has been unexpectedly determined that utilizing the recombinant proteins described herein at the elevated amounts (2 or 3-fold or more above that found in a biological sample), still allows for the recombinant proteins to work and interact as needed with their biological targets to exert the required or desired effects. Increasing the amount of one or more recombinant proteins above that found in nature, while still allowing for the proteins to function as required, without causing deleterious side effects, was an unexpected finding. Similarly, modifying the ratios of the recombinant proteins in a manner that is different from that found in nature, while still maintaining a desired biological outcome, was also unexpected.

In still further embodiments, provided herein are bio-mimetic formulations consisting of a carrier and 1-51, 1-50, 5-50, 10-50, or 10-40, or 20-45, or 20-40, or 20-30, or 2, 3, 4, 5, 10, 15, 20, 25, 30, 35, 40, 45 or 50 of the recombinant peptides recited in Table 1.

Still additional embodiments provide a bio-mimetic formulation comprising a carrier and the following recombinant peptides. Table 6 separates the recombinant proteins in to similar “classes” or “types” of recombinant proteins.

TABLE 6
Select Recombinant Proteins for Bio-Mimetic Formulations

at least one of:
Basic fibroblast growth factor (bFGF) at about 4440 (pg/mL) to about
44400 (pg/mL); or
Epidermal growth factor (EGF) at about 16.4 (pg/mL) to about 164
(pg/mL); or
Granulocyte colony-stimulating factor (GCSF) at about 144 (pg/mL) to
about 1440 (pg/mL); and
at least one of:
Platelet-derived growth factor (PDGF-AA) at about 34327 (pg/mL) to
about 343270 (pg/mL); or
Platelet-derived growth factor (PDGF-BB) at about 106 (pg/mL) to
about 1060 (pg/mL); or
Placental growth factor (PLGF) at about 370 (pg/mL) to about 3700
(pg/mL); and
at least one of:
Transforming growth factor alpha (TGF) - alpha at about 3.4 (pg/mL) to
about 34 (pg/mL); or
Transforming growth factor beta 1 (TGF-B1) at about 1180 (pg/mL) to
about 11800 (pg/mL); and
at least one of:
interleukin 4 (IL-4) at about 2.2 (pg/mL) to about 22 (pg/mL); or
interleukin 6 (IL-6) at about 74 (pg/mL) to about 740 (pg/mL); or
interleukin 8 (IL-8) at about 2875 (pg/mL) to about 28750 (pg/mL); or
interleukin 10 (IL-10) at about 4.1 (pg/mL) to about 41 (pg/mL); and
at least one of:
Tissue inhibitor of metalloproteinase (TIMP-1) at about 16630 (pg/mL)
to about 166300 (pg/mL); or
Tissue inhibitor of metalloproteinase (TIMP-2) at about 2960 (pg/mL)
to about 29600 (pg/mL); or
Tissue inhibitor of metalloproteinase (TIMP-4) at about 111 (pg/mL) to
about 1110 (pg/mL); and
at least one of:
Growth Differentiation Factor (GDF-15) at about 81.25 (pg/mL) to
about 812.5 (pg/mL); or
Granulocyte macrophage colony-stimulating factor (GM-CSF) at about
0.21 (pg/mL) to about 2.1 (pg/mL); and
Interferon gamma (IFNγ) at about 2.75 (pg/mL) to about 27.5 (pg/mL); and
at least one of:
Interleukin 1 alpha (IL1-alpha) at about 12.5 (pg/mL) to about 125
(pg/mL); or
Interleukin 1 Beta (IFN-β) at about 27.8 (pg/mL) to about 278 (pg/mL);
or
Interleukin 1 receptor antagonist (IL-1ra) at about 78.5 (pg/mL) to about
785 (pg/mL); or
Interleukin 5 (IL-5) at about 2.88 (pg/mL) to about 28.8 (pg/mL); or
Interleukin 7 (IL-7) at about 1.37 (pg/mL) to about 13.7 (pg/mL); or
Interleukin 12 p40 (IL-12p40) at about 9.55 (pg/mL) to about 95.5
(pg/mL); or
Interleukin 12 p70 (IL-12p70) at about 0.79 (pg/mL) to about 7.9
(pg/mL); or
Interleukin 15 (IL-15) at about 1.35 (pg/mL) to about 13.5 (pg/mL); or
Interleukin 17 (IL-17) at about 1.1 (pg/mL) to about 11 (pg/mL); or
Interleukin 16 (IL-16) at about 34.4 (pg/mL) to about 344(pg/mL); and
Macrophage colony- stimulating factor (MCSF) at about 4.3 (pg/mL) to about
43 (pg/mL); and
Osteoprotegerin (OPG) at about 319.69 (pg/mL) to about 3196.9 (pg/mL); and
B lymphocyte chemoattractant (CXCL13) (BLC) at about 60 (pg/mL) to about
600 (pg/mL); and
Chemokine ligand 1 (CCL1) (I-309) at about 4 (pg/mL) to about 40 (pg/mL);
and
Eotaxin-2 at about 0.13 (pg/mL) to about 1.3 (pg/mL); and
Monocyte chemotactic protein 1 (CCL2) (MCP-1) at about 76.95 (pg/mL) to
about 769.5 (pg/mL); and
Monokine induced by gamma interferon (CXCL9) (MIG) at about 780 (pg/mL)
to about 7800 (pg/mL); and
at least one of:
Macrophage inflammatory protein 1 alpha (CCL3) (MIP-1α) at about
13.08 (pg/mL) to about 130.8 (pg/mL); or
Macrophage inflammatory protein 1 beta (CCL4) (MIP-1β) at about
6.56 (pg/mL) to about 65.6 (pg/mL); or
Macrophage inflammatory protein ID (MIP-5, CCL15) (MIP-1d) at
about 4.3 (pg/mL) to about 43 (pg/mL); and
Regulated on activation, normal T cell expressed and secreted (CCL5)
(RANTES) at about 203 (pg/mL) to about 2030 (pg/mL); and
Brain-derived neurotrophic factor (BDNF) at about 45.03 (pg/mL) to about
450.3 (pg/mL); and
Bone morphogenetic protein 5 (BMP-5) at about 90.55 (pg/mL) to about 905.5
(pg/mL); and
Endocrine gland- derived vascular endothelial growth factor (EG-VEGF) at
about 496.68 (pg/mL) to about 4966.8 (pg/mL); and
Fibroblast growth factor 4 (FGF-4) at about 353.37 (pg/mL) to about 3533.7
(pg/mL); and
Keratinocyte growth factor (FGF-7) at about 46.7 (pg/mL) to about 467
(pg/mL); and
Growth hormone (GH) at about 114.23 (pg/mL) to about 1142.3 (pg/mL); and
at least one of:
Insulin-like growth factor (IGF-I) at about 27.65 (pg/mL) to about 276.5
(pg/mL); or
Insulin-like growth factor binding protein - 1 (IGFBP-1) at about
353.51 (pg/mL) to about 3535.1 (pg/mL); or
Insulin-like growth factor binding protein - 2 (IGFBP-2) at about
1072.52 (pg/mL) to about 10725.2 (pg/mL); or
Insulin-like growth factor binding protein - 3 (IGFBP-3) at about
7701.19 (pg/mL) to about 77011.9 (pg/mL); or
Insulin-like growth factor binding protein - 4 (IGFBP-4) at about
2954.34 (pg/mL) to about 29543.4 (pg/mL); or
Insulin-like growth factor binding protein - 6 (IGFBP-6) at about
5162.16 (pg/mL) to about 51621.6 (pg/mL)

In embodiments of the bio-mimetic formulations set forth in Table 5, the formulations can include at least 2, 3, 4, 5, etc. of the recited classes of recombinant proteins. Various combinations of the recited classes can also be prepared, for example 1, 2, 3, 4 or 5, of one class of recombinant protein, in combination with 1, 2, 3, 4 or 5, etc., of another class, or other classes, of recombinant proteins.

In further embodiments, the bio-mimetic formulations which include various combinations of classes of recombinant proteins can include combinations from Table 7 and/or Table 8, set forth below:

TABLE 7
Select Recombinant Proteins for Bio-Mimetic Formulations (Low Range)

at least one of:
Basic fibroblast growth factor (bFGF) at about 4440 (pg/mL) to about
13320 (pg/mL); or
Epidermal growth factor (EGF) at about 16.4 (pg/mL) to about 49.2
(pg/mL); or
Granulocyte colony-stimulating factor (GCSF) at about 144 (pg/mL) to
about 432 (pg/mL); and
at least one of:
Platelet-derived growth factor (PDGF-AA) at about 34327 (pg/mL) to
about 102981 (pg/mL); or
Platelet-derived growth factor (PDGF-BB) at about 106 (pg/mL) to
about 318 (pg/mL); or
Placental growth factor (PLGF) at about 370 (pg/mL) to about 1110
(pg/mL); and
at least one of:
Transforming growth factor alpha (TGF) - alpha at about 3.4 (pg/mL) to
about 10.2 (pg/mL); or
Transforming growth factor beta 1 (TGF-B1) at about 1180 (pg/mL) to
about 3540 (pg/mL); and
at least one of:
interleukin 4 (IL-4) at about 2.2 (pg/mL) to about 6.6 (pg/mL); or
interleukin 6 (IL-6) at about 74 (pg/mL) to about 222 (pg/mL); or
interleukin 8 (IL-8) at about 2875 (pg/mL) to about 8625 (pg/mL); or
interleukin 10 (IL-10) at about 4.1 (pg/mL) to about 12.3 (pg/mL); and
at least one of:
Tissue inhibitor of metalloproteinase (TIMP-1) at about 16630 (pg/mL)
to about 49890 (pg/mL); or
Tissue inhibitor of metalloproteinase (TIMP-2) at about 2960 (pg/mL)
to about 8880 (pg/mL); or
Tissue inhibitor of metalloproteinase (TIMP-4) at about 111 (pg/mL) to
about 333 (pg/mL); and
at least one of:
Growth Differentiation Factor (GDF-15) at about 81.25 (pg/mL) to
about 243.75 (pg/mL); or
Granulocyte macrophage colony-stimulating factor (GM-CSF) at about
0.21 (pg/mL) to about 0.63 (pg/mL); and
Interferon gamma (IFNγ) at about 2.75 (pg/mL) to about 8.25 (pg/mL); and
at least one of:
Interleukin 1 alpha (IL1-alpha) at about 12.5 (pg/mL) to about 37.5
(pg/mL); or
Interleukin 1 Beta (IFN-β) at about 27.8 (pg/mL) to about 83.4 (pg/mL);
or
Interleukin 1 receptor antagonist (IL-1ra) at about 78.5 (pg/mL) to about
235.5 (pg/mL); or
Interleukin 5 (IL-5) at about 2.88 (pg/mL) to about 8.64 (pg/mL); or
Interleukin 7 (IL-7) at about 1.37 (pg/mL) to about 4.11 (pg/mL); or
Interleukin 12 p40 (IL-12p40) at about 9.55 (pg/mL) to about 28.65
(pg/mL); or
Interleukin 12 p70 (IL-12p70) at about 0.79 (pg/mL) to about 2.37
(pg/mL); or
Interleukin 15 (IL-15) at about 1.35 (pg/mL) to about 4.05 (pg/mL); or
Interleukin 17 (IL-17) at about 1.1 (pg/mL) to about 3.3 (pg/mL); or
Interleukin 16 (IL-16) at about 34.4 (pg/mL) to about 103.2 (pg/mL); and
Macrophage colony- stimulating factor (MCSF) at about 4.3 (pg/mL) to about
12.9 (pg/mL); and
Osteoprotegerin (OPG) at about 319.69 (pg/mL) to about 959.07 (pg/mL); and
B lymphocyte chemoattractant (CXCL13) (BLC) at about 60 (pg/mL) to about
180 (pg/mL); and
Chemokine ligand 1 (CCL1) (I-309) at about 4 (pg/mL) to about 12 (pg/mL);
and
Eotaxin-2 at about 0.13 (pg/mL) to about 0.39 (pg/mL); and
Monocyte chemotactic protein 1 (CCL2) (MCP-1) at about 76.95 (pg/mL) to
about 230.85 (pg/mL); and
Monokine induced by gamma interferon (CXCL9) (MIG) at about 780 (pg/mL)
to about 2340 (pg/mL); and
at least one of:
Macrophage inflammatory protein 1 alpha (CCL3) (MIP-1α) at about
13.08 (pg/mL) to about 39.24(pg/mL); or
Macrophage inflammatory protein 1 beta (CCL4) (MIP-1β) at about
6.56 (pg/mL) to about 19.68 (pg/mL); or
Macrophage inflammatory protein 1D (MIP-5, CCL15) (MIP-1d) at
about 4.3 (pg/mL) to about 12.9 (pg/mL); and
Regulated on activation, normal T cell expressed and secreted (CCL5)
(RANTES) at about 203 (pg/mL) to about 609 (pg/mL); and
Brain-derived neurotrophic factor (BDNF) at about 45.03 (pg/mL) to about
135.09 (pg/mL); and
Bone morphogenetic protein 5 (BMP-5) at about 90.55 (pg/mL) to about 271.65
(pg/mL); and
Endocrine gland- derived vascular endothelial growth factor (EG-VEGF) at
about 496.68 (pg/mL) to about 1490.04 (pg/mL); and
Fibroblast growth factor 4 (FGF-4) at about 353.37 (pg/mL) to about 1060.11
(pg/mL); and
Keratinocyte growth factor (FGF-7) at about 46.7 (pg/mL) to about 140.1
(pg/mL); and
Growth hormone (GH) at about 114.23 (pg/mL) to about 342.69 (pg/mL); and
at least one of:
Insulin-like growth factor (IGF-I) at about 27.65 (pg/mL) to about 82.95
(pg/mL); or
Insulin-like growth factor binding protein - 1 (IGFBP-1) at about
353.51 (pg/mL) to about 1060.53 (pg/mL); or
Insulin-like growth factor binding protein - 2 (IGFBP-2) at about
1072.52 (pg/mL) to about 3217.56 (pg/mL); or
Insulin-like growth factor binding protein - 3 (IGFBP-3) at about
7701.19 (pg/mL) to about 23103.57 (pg/mL); or
Insulin-like growth factor binding protein - 4 (IGFBP-4) at about
2954.34 (pg/mL) to about 8863.02 (pg/mL); or
Insulin-like growth factor binding protein - 6 (IGFBP-6) at about
5162.16 (pg/mL) to about 15486.48 (pg/mL)

TABLE 8
Select Recombinant Proteins for Bio-
Mimetic Formulations (High Range)

at least one of:
Basic fibroblast growth factor (bFGF) at about 4440 (pg/mL) to about
44400 (pg/mL); or
Epidermal growth factor (EGF) at about 16.4 (pg/mL) to about 164
(pg/mL); or
Granulocyte colony-stimulating factor (GCSF) at about 144 (pg/mL) to
about 1440 (pg/mL); and
at least one of:
Platelet-derived growth factor (PDGF-AA) at about 34327 (pg/mL) to
about 343270 (pg/mL); or
Platelet-derived growth factor (PDGF-BB) at about 106 (pg/mL) to
about 1060 (pg/mL); or
Placental growth factor (PLGF) at about 370 (pg/mL) to about 3700
(pg/mL); and
at least one of:
Transforming growth factor alpha (TGF) - alpha at about 3.4 (pg/mL) to
about 34 (pg/mL); or
Transforming growth factor beta 1 (TGF-B1) at about 1180 (pg/mL) to
about 11800 (pg/mL); and
at least one of:
interleukin 4 (IL-4) at about 2.2 (pg/mL) to about 22 (pg/mL); or
interleukin 6 (IL-6) at about 74 (pg/mL) to about 740 (pg/mL); or
interleukin 8 (IL-8) at about 2875 (pg/mL) to about 28750 (pg/mL); or
interleukin 10 (IL-10) at about 4.1 (pg/mL) to about 41 (pg/mL); and
at least one of:
Tissue inhibitor of metalloproteinase (TIMP-1) at about 16630 (pg/mL)
to about 166300 (pg/mL); or
Tissue inhibitor of metalloproteinase (TIMP-2) at about 2960 (pg/mL)
to about 29600 (pg/mL); or
Tissue inhibitor of metalloproteinase (TIMP-4) at about 111 (pg/mL) to
about 1110 (pg/mL); and
at least one of:
Growth Differentiation Factor (GDF-15) at about 81.25 (pg/mL) to
about 812.5 (pg/mL); or
Granulocyte macrophage colony-stimulating factor (GM-CSF) at about
0.21 (pg/mL) to about 2.1 (pg/mL); and
Interferon gamma (IFNγ) at about 2.75 (pg/mL) to about 27.5 (pg/mL);
and
at least one of:
Interleukin 1 alpha (IL1-alpha) at about 12.5 (pg/mL) to about 125
(pg/mL); or
Interleukin 1 Beta (IFN-β) at about 27.8 (pg/mL) to about 278 (pg/mL);
or
Interleukin 1 receptor antagonist (IL-1ra) at about 78.5 (pg/mL) to about
785 (pg/mL); or
Interleukin 5 (IL-5) at about 2.88 (pg/mL) to about 28.8 (pg/mL); or
Interleukin 7 (IL-7) at about 1.37 (pg/mL) to about 13.7 (pg/mL); or
Interleukin 12 p40 (IL-12p40) at about 9.55 (pg/mL) to about 95.5
(pg/mL); or
Interleukin 12 p70 (IL-12p70) at about 0.79 (pg/mL) to about 7.9
(pg/mL); or
Interleukin 15 (IL-15) at about 1.35 (pg/mL) to about 13.5 (pg/mL); or
Interleukin 17 (IL-17) at about 1.1 (pg/mL) to about 11 (pg/mL); or
Interleukin 16 (IL-16) at about 34.4 (pg/mL) to about 344(pg/mL); and
Macrophage colony- stimulating factor (MCSF) at about 4.3 (pg/mL) to
about 43 (pg/mL); and
Osteoprotegerin (OPG) at about 319.69 (pg/mL) to about 3196.9 (pg/mL);
and
B lymphocyte chemoattractant (CXCL13) (BLC) at about 60 (pg/mL) to
about 600 (pg/mL); and
Chemokine ligand 1 (CCL1) (I-309) at about 4 (pg/mL) to about 40
(pg/mL); and
Eotaxin-2 at about 0.13 (pg/mL) to about 1.3 (pg/mL); and
Monocyte chemotactic protein 1 (CCL2) (MCP-1) at about 76.95 (pg/mL)
to about 769.5 (pg/mL); and
Monokine induced by gamma interferon (CXCL9) (MIG) at about 780
(pg/mL) to about 7800 (pg/mL); and
at least one of:
Macrophage inflammatory protein 1 alpha (CCL3) (MIP-1α) at about
13.08 (pg/mL) to about 130.8 (pg/mL); or
Macrophage inflammatory protein 1 beta (CCL4) (MIP-1β) at about
6.56 (pg/mL) to about 65.6 (pg/mL); or
Macrophage inflammatory protein 1D (MIP-5, CCL15) (MIP-1d) at
about 4.3 (pg/mL) to about 43 (pg/mL); and
Regulated on activation, normal T cell expressed and secreted (CCL5)
(RANTES) at about 203 (pg/mL) to about 2030 (pg/mL); and
Brain-derived neurotrophic factor (BDNF) at about 45.03 (pg/mL) to
about 450.3 (pg/mL); and
Bone morphogenetic protein 5 (BMP-5) at about 90.55 (pg/mL) to about
905.5 (pg/mL); and
Endocrine gland- derived vascular endothelial growth factor (EG-VEGF)
at about 496.68 (pg/mL) to about 4966.8 (pg/mL); and
Fibroblast growth factor 4 (FGF-4) at about 353.37 (pg/mL) to about
3533.7 (pg/mL); and
Keratinocyte growth factor (FGF-7) at about 46.7 (pg/mL) to about 467
(pg/mL); and
Growth hormone (GH) at about 114.23 (pg/mL) to about 1142.3 (pg/mL);
and
at least one of:
Insulin-like growth factor (IGF-I) at about 27.65 (pg/mL) to about
276.5 (pg/mL); or
Insulin-like growth factor binding protein - 1 (IGFBP-1) at about
353.51 (pg/mL) to about 3535.1 (pg/mL); or
Insulin-like growth factor binding protein - 2 (IGFBP-2) at about
1072.52 (pg/mL) to about 10725.2 (pg/mL); or
Insulin-like growth factor binding protein - 3 (IGFBP-3) at about
7701.19 (pg/mL) to about 77011.9 (pg/mL); or
Insulin-like growth factor binding protein - 4 (IGFBP-4) at about
2954.34 (pg/mL) to about 29543.4 (pg/mL); or
Insulin-like growth factor binding protein - 6 (IGFBP-6) at about
5162.16 (pg/mL) to about 51621.6 (pg/mL)

In additional embodiments, provided herein are methods of preparing the various bio-mimetic formulations disclosed throughout. In embodiments, the methods include obtaining the various combinations and mounts of the recombinant proteins, mixing the recombinant proteins in a suitable carrier, and storing the bio-mimetic formulation in a container.

In embodiments that utilize a liquid carrier, the carrier components are suitably prepared and mixed, and then the recombinant peptides are added according to the desired amounts and various combinations. The use of suitable buffer systems and stabilizers can allow the bio-mimetic formulations to be stored in a glass, plastic, metal, etc., container for periods of at least about 30 days at about 4-8° C. In other embodiments, the bio-mimetic formulations can be stored for at least about 60 days, at least about 90 days, at least about 120 days, at least about 240 days, at least about 1 year, or about 1-3 years, about 1-5 years, etc., at 4-8° C. In other embodiments, the buffer and stabilizers can be selected so as to allow for extended storage (90 days or more) at room temperature (about 20-25° C.), or frozen (below 0° C.).

As described herein, methods of producing recombinant proteins are known in the art, and in embodiments, the methods of preparation described herein include preparing the various peptides using vector systems, including viral plasmids, bacterial production systems, etc. to generate the recombinant proteins, followed by purification/filtration if necessary, prior to mixing with the various carrier systems.

In further embodiments, methods of preparing a dried, bio-mimetic formulation are provided. In such embodiments, various amounts and combinations of the recombinant proteins described herein are obtained and mixed with a liquid carrier. This liquid carrier can then be dried (i.e., the aqueous component removed), for example by using dehydration, lyophilization, desiccation, etc., resulting in a dried carrier formulation.

In other embodiments, a dried, bio-mimetic formulation can be prepared by obtaining various amounts and combinations of the recited recombinant proteins, and mixing into a liquid carrier. A solid scaffold matrix or extracellular matrix can also be prepared. The recombinant peptides in the carrier are then applied to the solid scaffold matrix. The liquid (e.g., water) in the carrier can then be removed, leaving a dried composition of the recombinant proteins on the solid scaffold matrix. Methods of drying the recombinant protein compositions onto the solid scaffold matrix include evaporation, dehydration, desiccation, lyophilization, freeze drying, etc.

As used herein “solid scaffold matrix” refers to a material capable of being formed into an appropriate shape to receive a liquid carrier and also be used for drying the bio-mimetic formulation, and can also be referred to as an extracellular matrix. In embodiments, the solid scaffold matrix can be an acetal resin engineering plastic, such as TEFLON® or DELRIN® (DuPont, Wilmington, Del.). Additional materials that can be used as a solid scaffold matrix include, but are not limited to, allograft pericardium, allograft acellular dermis, Wharton's jelly, purified xenograft Type-1 collagen, bio cellulose polymers or copolymers, biocompatible synthetic polymer or copolymer films, purified small intestinal submucosa, bladder acellular matrix, cadaveric fascia, or any combination thereof.

In exemplary embodiments, the solid scaffold matrix can include the following components, in addition to various additional structural components:

TABLE 9
Exemplary Components of Solid Scaffold Matrix

| Component | Range of Amount | Suitable Amount |
|---|---|---|
| MMP1 (Matrix | about 0.24 to about 2.4 | 0.42 ± 0.18 (moles |
| metalloproteinase-1) | (moles component/mg of | component/mg of solid |
| | solid scaffold matrix) | scaffold matrix) |
| MMP2 (Matrix | about 0.5 to about 5.0 | 0.94 ± 0.987 (moles |
| metalloproteinase-2) | (moles component/mg of | component/mg of solid |
| | solid scaffold matrix) | scaffold matrix) |
| MMP3 (Matrix | about 0.18 to about 2.8 | 0.47 ± 0.29 (moles |
| metalloproteinase-3) | (moles component/mg of | component/mg of solid |
| | solid scaffold matrix) | scaffold matrix) |
| MMP7 (Matrix | | |
| metalloproteinase-7) | | |
| MMP8 (Matrix | about 0.24 to about 2.4 | 1.03 ± 0.79 (moles |
| metalloproteinase-8) | (moles component/mg of | component/mg of solid |
| | solid scaffold matrix) | scaffold matrix) |
| MMP9 (Matrix | about 0.68 to about 6.8 | 2.67 ± 1.99 (moles |
| metalloproteinase-9) | (moles component/mg of | component/mg of solid |
| | solid scaffold matrix) | scaffold matrix) |
| MMP10 (Matrix | about 0.05 to about 0.5 | 0.10 ± 0.15 (moles |
| metalloproteinase-10) | (moles component/mg of | component/mg of solid |
| | solid scaffold matrix) | scaffold matrix) |
| MMP11 (Matrix | | |
| metalloproteinase-11) | | |
| MMP12 (Matrix | | |
| metalloproteinase-12) | | |
| MMP13 (Matrix | about 0.0092 to about | 0.01 ± 0.0008 (moles |
| metalloproteinase-13) | 0.092 (moles component/ | component/mg of solid |
| | mg of solid scaffold | scaffold matrix) |
| | matrix) | |
| MMP14 (Matrix | | |
| metalloproteinase-14) | | |
| MMP15 (Matrix | | |
| metalloproteinase-15) | | |
| MMP16 (Matrix | | |
| metalloproteinase-16) | | |
| MMP17 (Matrix | | |
| metalloproteinase-17) | | |
| MMP18 (Matrix | | |
| metalloproteinase-18) | | |
| MMP19 (Matrix | | |
| metalloproteinase-19) | | |
| MMP20 (Matrix | | |
| metalloproteinase-20) | | |
| MMP21 (Matrix | | |
| metalloproteinase-21) | | |
| MMP22 (Matrix | | |
| metalloproteinase-22) | | |
| MMP23 (Matrix | | |
| metalloproteinase-23) | | |
| MMP24 (Matrix | | |
| metalloproteinase-24) | | |
| MMP25 (Matrix | | |
| metalloproteinase-25) | | |
| MMP26 (Matrix | | |
| metalloproteinase-26) | | |
| MMP27 (Matrix | | |
| metalloproteinase-27) | | |
| MMP28 (Matrix | | |
| metalloproteinase-28) | | |
| Laminin | about 0.552 to about | 1.46 ± 0.908 (ng |
| | 5.552 (ng component/mg | component/mg of solid |
| | of solid scaffold matrix) | scaffold matrix) |
| Fibronectin | about 0.79 to about 7.9 | 1.84 ± 1.05 × 10³ (ng |
| | (ng component/mg of | component/mg of solid |
| | solid scaffold matrix) | scaffold matrix) |
| alpha 2 macroglobulin | about 37.85 to about | 92.18 ± 53.33 (moles |
| | 378.5 (moles component/ | component/mg of solid |
| | mg of solid scaffold | scaffold matrix) |
| | matrix) | |

In further embodiments, the bio-mimetic formulations which include various combinations of classes of recombinant proteins can include combinations from Table 9, set for the below, including the various ranges and amounts of the recombinant proteins listed below:

TABLE 9

| | | Exemplary | Low End | Upper End |
|---|---|---|---|---|
| Recombinant | | Amount | Value | Value |
| Protein | Expanded Name | (pg/mL) | (pg/mL) | (pg/mL) |
| ENA-78 | | 1.6 | 0.2 | 1608.3517 |
| GRO | | 0.6-1.7 | 0.1 | 1700 |
| IL-18 BPa | | 4.1 | 0.4 | 4081.5124 |
| IL-31 | | 0.2 | 0.1 | 196.84694 |
| I-TAC | | 4.2 | 0.4 | 4233.881 |
| LIF | | 3.4 | 0.3 | 3378.533 |
| LIGHT | | 2.5 | 0.3 | 2500.93 |
| Lymphotactin | | 23.9 | 2.4 | 23932.025 |
| MCP-2 | | 0.6 | 0.1 | 551.96996 |
| MCP-3 | | 0.6 | 0.1 | 609.13575 |
| MCP-4 | | 0.3 | 0.1 | 292.47905 |
| MIP-3a | | 0.2 | 0.1 | 229.96335 |
| MIP-3b | | 1.1-3.5 | 0.1 | 3500 |
| MPIF-1 | | 0.4 | 0.1 | 399.99117 |
| MSP | | 21.0 | 2.1 | 20958.324 |
| NAP-2 | | 0.7 | 0.1 | 689.13343 |
| OPN | | 0.8 | 0.1 | 756.41221 |
| PARC | | 0.6-7.3 | 0.1 | 7300 |
| PF4 | Platelet factor 4 (PF-4) | 0.2-433.4 | 0.1 | 433400 |
| | (C-X-C motif chemokine | | | |
| | 4) (Iroplact) (Oncostatin- | | | |
| | A) [Cleaved into: Platelet | | | |
| | factor 4, short form] | | | |
| SDF-1a | | 1.6 | 0.2 | 1562.9177 |
| TECK | | 9.4 | 0.9 | 9440.3426 |
| ADAM9 | Disintegrin and | 399.8 | 40.0 | 399782.39 |
| | metalloproteinase | | | |
| | domain-containing | | | |
| | protein 9 (ADAM 9) (EC | | | |
| | 3.4.24.—) (Cellular | | | |
| | disintegrin-related | | | |
| | protein) (Meltrin-gamma) | | | |
| | (Metalloprotease/disintegrin/ | | | |
| | cysteine-rich protein 9) | | | |
| | (Myeloma cell | | | |
| Cathepsin L | | 50.8 | 5.1 | 50758.58 |
| CD97 | CD97 antigen | 1,657.4 | 165.7 | 1657408.3 |
| | (Leukocyte antigen | | | |
| | CD97) (CD antigen | | | |
| | CD97) [Cleaved into: | | | |
| | CD97 antigen subunit | | | |
| | alpha; CD97 antigen | | | |
| | subunit beta] | | | |
| FAP | | 8.6 | 0.9 | 8578.3726 |
| Galectin-3 | Galectin-3 (Gal-3) (35 | 372.7 | 37.3 | 372658.09 |
| | kDa lectin) | | | |
| | (Carbohydrate-binding | | | |
| | protein 35) (CBP 35) | | | |
| | (Galactose-specific | | | |
| | lectin 3) (Galactoside- | | | |
| | binding protein) | | | |
| | (GALBP) (IgE-binding | | | |
| | protein) (L-31) (Laminin- | | | |
| | binding protein) (Lectin | | | |
| | L-29) | | | |
| IGF-2R | Cation-independent | 5,984.9 | 598.5 | 5984898.6 |
| | mannose-6-phosphate | | | |
| | receptor (CI Man-6-P | | | |
| | receptor) (CI-MPR) | | | |
| | (M6PR) (300 kDa | | | |
| | mannose 6-phosphate | | | |
| | receptor) (MPR 300) | | | |
| | (Insulin-like growth | | | |
| | factor 2 receptor) | | | |
| | (Insulin-like growth | | | |
| | factor II receptor) (IGF-II | | | |
| | receptor) (M6P/IGF2 | | | |
| | receptor) (M6P/IGF2R) | | | |
| | (CD antigen CD222) | | | |
| Neprilysin | Neprilysin (EC | 832.1 | 83.2 | 832141.8 |
| | 3.4.24.11) | | | |
| | (Atriopeptidase) | | | |
| | (Common acute | | | |
| | lymphocytic leukemia | | | |
| | antigen) (CALLA) | | | |
| | (Enkephalinase) (Neutral | | | |
| | endopeptidase 24.11) | | | |
| | (NEP) (Neutral | | | |
| | endopeptidase) (Skin | | | |
| | fibroblast elastase) | | | |
| | (SFE) (CD antigen | | | |
| | CD10) | | | |
| NOV | | 2.5 | 0.2 | 2458.9006 |
| PGRP-S | Peptidoglycan | 9.4 | 0.9 | 9359.735 |
| | recognition protein 1 | | | |
| | (Peptidoglycan | | | |
| | recognition protein short) | | | |
| | (PGRP-S) | | | |
| Serpin A4 | Kallistatin (Kallikrein | 4,640.8 | 464.1 | 4640831.7 |
| | inhibitor) (Peptidase | | | |
| | inhibitor 4) (PI-4) (Serpin | | | |
| | A4) | | | |
| TLR2 | Toll-like receptor 2 | 122.0 | 12.2 | 122039.83 |
| | (Toll/interleukin-1 | | | |
| | receptor-like protein 4) | | | |
| | (CD antigen CD282) | | | |
| Transferrin | Serotransferrin | 4625.7-16238.9 | 462.6 | 16238900 |
| | (Transferrin) (Beta-1 | | | |
| | metal-binding globulin) | | | |
| | (Siderophilin) | | | |
| Albumin | Serum albumin | 239-540.9 | 23.9 | 540900 |
| CA19-9 | Carbohydrate antigen 19- | 1,511.4 | 151.1 | 1511427.3 |
| | 9, also called cancer | | | |
| | antigen 19-9, or sialylated | | | |
| | Lewis (a) antigen | | | |
| CD163 | | 8.4 | 0.8 | 8373.8074 |
| Cystatin C | | 80.6 | 8.1 | 80573.142 |
| Decorin | Decorin (Bone | 887.1 | 88.7 | 887080.79 |
| | proteoglycan II) (PG-S2) | | | |
| | (PG40) | | | |
| Dkk-3 | Dickkopf-related protein 3 | 43.1 | 4.3 | 43053.353 |
| | (Dickkopf-3) (Dkk-3) (hDkk- | | | |
| | 3) | | | |
| Fetuin A | Alpha-2-HS-glycoprotein | 225.6-199374 | 22.6 | 199374000 |
| | (Alpha-2-Z-globulin) (Ba- | | | |
| | alpha-2-glycoprotein) | | | |
| | (Fetuin-A) [Cleaved into: | | | |
| | Alpha-2-HS-glycoprotein | | | |
| | chain A; Alpha-2-HS- | | | |
| | glycoprotein chain B] | | | |
| FOLR1 | Folate receptor alpha (FR- | 57.6 | 5.8 | 57601.162 |
| | alpha) (Adult folate- | | | |
| | binding protein) (FBP) | | | |
| | (Folate receptor 1) (Folate | | | |
| | receptor, adult) (KB cells | | | |
| | FBP) (Ovarian tumor- | | | |
| | associated antigen | | | |
| | MOv18) | | | |
| HAI-2 | | 7.8 | 0.8 | 7764.0163 |
| IL-27 | | 1.4 | 0.1 | 1411.1913 |
| LAG-3 | | 20.6 | 2.1 | 20627.085 |
| RBP4 | Retinol-binding protein 4 | 1654.5-5745.2 | 165.5 | 5745200 |
| | (Plasma retinol-binding | | | |
| | protein) (PRBP) (RBP) | | | |
| | [Cleaved into: Plasma | | | |
| | retinol-binding protein(1- | | | |
| | 182); Plasma retinol- | | | |
| | binding protein(1-181); | | | |
| | Plasma retinol-binding | | | |
| | protein(1-179); Plasma | | | |
| | retinol-binding protein(1- | | | |
| | 176)] | | | |
| TACI | Tumor necrosis factor | 141.3 | 14.1 | 141318.11 |
| | receptor superfamily | | | |
| | member 13B | | | |
| | (Transmembrane activator | | | |
| | and CAML interactor) (CD | | | |
| | antigen CD267) | | | |
| Adiponectin | | 244.6 | 24.5 | 244589.78 |
| ANGPTL4 | | 33.0 | 3.3 | 32962.016 |
| B2M | Beta-2-microglobulin | 68.6-520.1 | 6.9 | 520100 |
| | [Cleaved into: Beta-2- | | | |
| | microglobulin form pI | | | |
| | 5.3] | | | |
| BCAM | | 19.2 | 1.9 | 19230.662 |
| CA125 | Mucin-16 (MUC-16) | 108.8-138194.8 | 10.9 | 138194000 |
| | (Ovarian cancer-related | | | |
| | tumor marker CA125) | | | |
| | (CA-125) (Ovarian | | | |
| | carcinoma antigen | | | |
| | CA125) | | | |
| CEA | Carcinoembryonic antigen | 102.5 | 10.2 | 102497.22 |
| | (CEA). In humans, the | | | |
| | carcinoembryonic antigen | | | |
| | family consists of 29 | | | |
| | genes, 18 of which are | | | |
| | normally expressed. | | | |
| CRP | C-reactive protein [Cleaved | 10.6 | 1.1 | 10624.726 |
| | into: C-reactive protein(1- | | | |
| | 205)] | | | |
| Ferritin | | 635.9 | 63.6 | 635916.3 |
| FSH | | 7.1 | 0.7 | 7136.6774 |
| hCGb | | 22.5 | 2.3 | 22543.625 |
| IL-3 | | 15.6 | 1.6 | 15602.928 |
| IL-21 | | 324.6 | 32.5 | 324558.31 |
| Leptin | | 9.8 | 1.0 | 9811.1399 |
| MMP-1 | | 38.3 | 3.8 | 38265.885 |
| MMP-2 | 72 kDa type IV | 493.4 | 49.3 | 493442.5 |
| | collagenase (EC | | | |
| | 3.4.24.24) (72 kDa | | | |
| | gelatinase) (Gelatinase | | | |
| | A) (Matrix | | | |
| | metalloproteinase-2) | | | |
| | (MMP-2) (TBE-1) | | | |
| | [Cleaved into: PEX] | | | |
| MMP-3 | | 16.9 | 1.7 | 16884.21 |
| MMP-8 | | 14.7-18.9 | 1.5 | 18900 |
| MMP-9 | Matrix metalloproteinase- | 33.2 | 3.3 | 33175.025 |
| | 9 (MMP-9) (EC 3.4.24.35) | | | |
| | (92 kDa gelatinase) (92 | | | |
| | kDa type IV collagenase) | | | |
| | (Gelatinase B) (GELB) | | | |
| | [Cleaved into: 67 kDa | | | |
| | matrix metalloproteinase- | | | |
| | 9; 82 kDa matrix | | | |
| | metalloproteinase-9] | | | |
| MMP-10 | Stromelysin-2 (SL-2) (EC | 0.5-1.3 | 0.1 | 1300 |
| | 3.4.24.22) (Matrix | | | |
| | metalloproteinase-10) | | | |
| | (MMP-10) (Transin-2) | | | |
| MMP-13 | Collagenase 3 (EC | 9.9-59.9 | 1.0 | 59900 |
| | 3.4.24.—) (Matrix | | | |
| | metalloproteinase-13) | | | |
| | (MMP-13) | | | |
| NCAM-1 | | 74.7 | 7.5 | 74728.399 |
| Nidogen-1 | Nidogen-1 (NID-1) | 10.5-10663.7 | 1.1 | 10663700 |
| | (Entactin) | | | |
| NSE | | 16.2 | 1.6 | 16204.701 |
| OSM | | 19.6 | 2.0 | 19589.185 |
| Prolactin | | 444.3 | 44.4 | 444334.55 |
| PSA-free | | 16.9 | 1.7 | 16933.554 |
| Siglec-9 | | 18.8 | 1.9 | 18778.24 |
| TACE | Disintegrin and | 17.7-174.8 | 1.8 | 174800 |
| | metalloproteinase | | | |
| | domain-containing | | | |
| | protein 17 (ADAM 17) | | | |
| | (EC 3.4.24.86) (Snake | | | |
| | venom-like protease) | | | |
| | (TNF-alpha convertase) | | | |
| | (TNF-alpha-converting | | | |
| | enzyme) (CD antigen | | | |
| | CD156b) | | | |
| TIMP-4 | | 5.9 | 0.6 | 5910.0444 |
| Activin A | Inhibin beta A chain | 366.6 | 36.7 | 366604.67 |
| | (Activin beta-A chain) | | | |
| | (Erythroid differentiation | | | |
| | protein) (EDF) | | | |
| AgRP | | 1.2 | 0.1 | 1235.4788 |
| Angiogenin | Angiogenin (EC 3.1.27.—) | 4.6-64.4 | 0.5 | 64400 |
| | (Ribonuclease 5) | | | |
| | (RNase 5) | | | |
| ANG-1 | | 10.1 | 1.0 | 10092.204 |
| Angiostatin | Plasminogen (EC | 413.3-5376.2 | 41.3 | 5376000 |
| | 3.4.21.7) [Cleaved into: | | | |
| | Plasmin heavy chain A; | | | |
| | Activation peptide; | | | |
| | Angiostatin; Plasmin | | | |
| | heavy chain A, short | | | |
| | form; Plasmin light chain | | | |
| | B] | | | |
| Cathepsin S | Cathepsin S (EC | 9.6-10.4 | 1.0 | 10400 |
| | 3.4.22.27) | | | |
| CD40 | | 15.0 | 1.5 | 14967.535 |
| Cripto-1 | | 5.0 | 0.5 | 4975.1248 |
| DAN | | 105.1 | 10.5 | 105061.11 |
| DKK-1 | | 27.1 | 2.7 | 27128.196 |
| E-Cadherin | | 141.7 | 14.2 | 141675.42 |
| EpCAM | | 3.2 | 0.3 | 3198.2786 |
| FAS L | Tumor necrosis factor | 30.7 | 3.1 | 30735.803 |
| | ligand superfamily | | | |
| | member 6 (Apoptosis | | | |
| | antigen ligand) (APTL) | | | |
| | (CD95 ligand) (CD95-L) | | | |
| | (Fas antigen ligand) | | | |
| | (Fas ligand) (FasL) (CD | | | |
| | antigen CD178) | | | |
| | [Cleaved into: Tumor | | | |
| | necrosis factor ligand | | | |
| Fcg RIIBC | Low affinity | 81.1 | 8.1 | 81141.464 |
| | immunoglobulin gamma | | | |
| | Fc region receptor II-b | | | |
| | (IgG Fc receptor II-b) | | | |
| | (CDw32) (Fc-gamma | | | |
| | RII-b) (Fc-gamma-RIIb) | | | |
| | (FcRII-b) (CD antigen | | | |
| | CD32); Low affinity | | | |
| | immunoglobulin gamma | | | |
| | Fc region receptor II-c | | | |
| Follistatin | | 1.8-12.5 | 1.7 | 12500 |
| Galectin-7 | Galectin-7 (Gal-7) (HKL- | 38.8-292.4 | 3.9 | 292400 |
| | 14) (PI7) (p53-induced | | | |
| | gene 1 protein) | | | |
| ICAM-2 | Intercellular adhesion | 149.3 | 14.9 | 149308.12 |
| | molecule 2 (ICAM-2) | | | |
| | (CD antigen CD102) | | | |
| IL-13 R1 | | 1.4-6.3 | 0.1 | 6300 |
| IL-13 R2 | Interleukin-13 receptor | 47.4-209.0 | 4.7 | 209000 |
| | subunit alpha-2 (IL-13 | | | |
| | receptor subunit alpha- | | | |
| | 2) (IL-13R subunit alpha- | | | |
| | 2) (IL-13R-alpha-2) (IL- | | | |
| | 13RA2) (Interleukin-13- | | | |
| | binding protein) (CD | | | |
| | antigen CD213a2) | | | |
| IL-17B | | 5.2 | 0.5 | 5206.2476 |
| IL-2 Ra | | 6.0 | 0.6 | 6040.3327 |
| IL-23 | | 2.7-34.8 | 0.3 | 34800 |
| LAP(TGFb1) | Human TGF-beta 1 | 0.1-52.7 | 0.1 | 52700 |
| | cDNA encodes a 390 | | | |
| | amino acid (aa) | | | |
| | precursor that contains a | | | |
| | 29 aa signal peptide and | | | |
| | a 361 aa proprotein, | | | |
| | which is further cleaved | | | |
| | to an N-terminal 249 aa | | | |
| | latency-associated | | | |
| | peptide (LAP) and | | | |
| NrCAM | Neuronal cell adhesion | 31.4 | 3.1 | 31375.447 |
| | molecule (Nr-CAM) | | | |
| | (Neuronal surface | | | |
| | protein Bravo) (hBravo) | | | |
| | (NgCAM-related cell | | | |
| | adhesion molecule) (Ng- | | | |
| | CAM-related) | | | |
| PAI-1 | Plasminogen activator | 0.6-2103.3 | 0.1 | 2103300 |
| | inhibitor 1 (PAI) (PAI-1) | | | |
| | (Endothelial | | | |
| | plasminogen activator | | | |
| | inhibitor) (Serpin E1) | | | |
| PDGF-AB | | 12.6 | 1.3 | 12601.852 |
| Resistin | | 29.6 | 3.0 | 29595.005 |
| SDF-1b | | 3.7 | 0.4 | 3688.2262 |
| gp130 | | 157.2 | 15.7 | 157247.2 |
| Shh-N | Sonic hedgehog protein | 23.9 | 2.4 | 23906.235 |
| | (SHH) (HHG-1) [Cleaved | | | |
| | into: Sonic hedgehog | | | |
| | protein N-product; Sonic | | | |
| | hedgehog protein C- | | | |
| | product] | | | |
| Siglec-5 | | 23.9 | 2.4 | 23926.46 |
| IL-1 R4 | | 7.1 | 0.7 | 7107.8063 |
| TGFb2 | | 27.5 | 2.8 | 27547.662 |
| Tie-2 | Angiopoietin-1 receptor | 76.8 | 7.7 | 76819.804 |
| | (EC 2.7.10.1) | | | |
| | (Endothelial tyrosine | | | |
| | kinase) (Tunica interna | | | |
| | endothelial cell kinase) | | | |
| | (Tyrosine kinase with Ig | | | |
| | and EGF homology | | | |
| | domains-2) (Tyrosine- | | | |
| | protein kinase receptor | | | |
| | TEK) (Tyrosine-protein | | | |
| | kinase receptor TIE-2) | | | |
| | (hTIE2) (p140 TEK) (CD | | | |
| | antigen CD202b) | | | |
| TPO | | 67.4 | 6.7 | 67405.385 |
| TRAIL R4 | Tumor necrosis factor | 57.8 | 5.8 | 57815.572 |
| | receptor superfamily | | | |
| | member 10D (Decoy | | | |
| | receptor 2) (DcR2) | | | |
| | (TNF-related apoptosis- | | | |
| | inducing ligand receptor | | | |
| | 4) (TRAIL receptor 4) | | | |
| | (TRAIL-R4) (TRAIL | | | |
| | receptor with a truncated | | | |
| | death domain) | | | |
| TREM-1 | | 24.0 | 2.4 | 23962.94 |
| VEGF-C | | 11.7 | 1.2 | 11670.245 |
| VEGF R1 | Vascular endothelial | 59.4-444.7 | 5.9 | 444700 |
| | growth factor receptor 1 | | | |
| | (VEGFR-1) (EC | | | |
| | 2.7.10.1) (Fms-like | | | |
| | tyrosine kinase 1) (FLT- | | | |
| | 1) (Tyrosine-protein | | | |
| | kinase FRT) (Tyrosine- | | | |
| | protein kinase receptor | | | |
| | FLT) (FLT) (Vascular | | | |
| | permeability factor | | | |
| ADAM8 | | 5.9 | 0.6 | 5937.2801 |
| B7-H3 | CD276 antigen (4Ig-B7- | 70.9-107.4 | 7.1 | 107400 |
| | H3) (B7 homolog 3) (B7- | | | |
| | H3) (Costimulatory | | | |
| | molecule) (CD antigen | | | |
| | CD276) | | | |
| Cadherin-13 | | 11.0 | 1.1 | 10951.828 |
| CD48 | | 233.3 | 23.3 | 233338.03 |
| CD99 | CD99 antigen (12E7) | 3.3-83.7 | 0.3 | 83700 |
| | (E2 antigen) (Protein | | | |
| | MIC2) (T-cell surface | | | |
| | glycoprotein E2) (CD | | | |
| | antigen CD99) | | | |
| CD229 | | 42.3 | 4.2 | 42331.572 |
| CEACAM-5 | Carcinoembryonic | 207.0-366.7 | 20.7 | 366700 |
| | antigen-related cell | | | |
| | adhesion molecule 5 | | | |
| | (Carcinoembryonic | | | |
| | antigen) (CEA) | | | |
| | (Meconium antigen 100) | | | |
| | (CD antigen CD66e) | | | |
| Cystatin A | | 1.4-2.5 | 0.1 | 2500 |
| Cystatin B | Cystatin-B (CPI-B) (Liver | 5.8-77.3 | 0.6 | 77300 |
| | thiol proteinase inhibitor) | | | |
| | (Stefin-B) | | | |
| Cystatin E M | Cystatin-M (Cystatin-6) | 2.6-247.9 | 0.5 | 247900 |
| | (Cystatin-E) | | | |
| Desmoglein 2 | | 0.5-12.8 | 0.1 | 12800 |
| DR3 | | 212.2 | 21.2 | 212172.13 |
| ErbB4 | | 1.1 | 0.1 | 1070.307 |
| ESAM | | 2.7 | 0.3 | 2735.6701 |
| FGF-21 | | 6.8 | 0.7 | 6824.9236 |
| Galectin-2 | Galectin-2 (Gal-2) (Beta- | 119.1 | 11.9 | 119140.34 |
| | galactoside-binding lectin | | | |
| | L-14-II) (HL14) (Lactose- | | | |
| | binding lectin 2) (S-Lac | | | |
| | lectin 2) | | | |
| Galectin-9 | Galectin-9 (Gal-9) | 18.8-367.6 | 1.9 | 367600 |
| | (Ecalectin) (Tumor antigen | | | |
| | HOM-HD-21) | | | |
| JAM-B | | 22.1 | 2.2 | 22105.271 |
| Kallikrein 5 | Kallikrein-5 (EC 3.4.21.—) | 129.4 | 12.9 | 129388.03 |
| | (Kallikrein-like protein 2) | | | |
| | (KLK-L2) (Stratum | | | |
| | corneum tryptic enzyme) | | | |
| Midkine | | 19.8-32 | 2.0 | 32000 |
| Pentraxin 3 | Pentraxin-related protein | 31.4-100.9 | 3.1 | 100900 |
| | PTX3 (Pentaxin-related | | | |
| | protein PTX3) (Tumor | | | |
| | necrosis factor alpha- | | | |
| | induced protein 5) (TNF | | | |
| | alpha-induced protein 5) | | | |
| | (Tumor necrosis factor- | | | |
| | inducible gene 14 | | | |
| | protein) (TSG-14) | | | |
| Pref-1 | | 554.8 | 55.5 | 554824.44 |
| Siglec-10 | | 266.8 | 26.7 | 266781.06 |
| SLAM | | 138.2 | 13.8 | 138165.88 |
| SP-D | Pulmonary surfactant- | 109.7 | 11.0 | 109685.54 |
| | associated protein D | | | |
| | (PSP-D) (SP-D) | | | |
| | (Collectin-7) (Lung | | | |
| | surfactant protein D) | | | |
| Syndecan-4 | Syndecan-4 (SYND4) | 3.4-77.9 | 0.3 | 77900 |
| | (Amphiglycan) | | | |
| | (Ryudocan core protein) | | | |
| Testican 2 | | 30.2 | 3.0 | 30239.317 |
| TIM-3 | | 25.4 | 2.5 | 25444.14 |
| TLR4 | | 11.7 | 1.2 | 11729.567 |
| TRAIL | | 4.2 | 0.4 | 4190.4283 |
| ULBP-1 | | 54.3 | 5.4 | 54340.852 |
| ADAMTS13 | | 38.7 | 3.9 | 38692.371 |
| Aggrecan | | 0.4 | 0.0 | 421.92648 |
| Angiotensinogen | | 32.7-368.6 | 3.3 | 368600 |
| B7-H1 | Programmed cell death | 69.8 | 7.0 | 69781.9 |
| | 1 ligand 1 (PD-L1) | | | |
| | (PDCD1 ligand 1) | | | |
| | (Programmed death | | | |
| | ligand 1) (B7 homolog 1) | | | |
| | (B7-H1) (CD antigen | | | |
| | CD274) | | | |
| BMPR-IA | | 92.0 | 9.2 | 92023.533 |
| BMPR-II | | 109.1 | 10.9 | 109051.17 |
| Cadherin-11 | | 3.6-4.5 | 0.4 | 4500 |
| CD27 | | 4.0 | 0.4 | 4013.197 |
| Ck beta 8-1 | | 1.5 | 0.2 | 1524.2229 |
| EMMPRIN | Basigin (5F7) | 21.0-1796.5 | 2.1 | 1796500 |
| | (Collagenase stimulatory | | | |
| | factor) (Extracellular | | | |
| | matrix metalloproteinase | | | |
| | inducer) (EMMPRIN) | | | |
| | (Leukocyte activation | | | |
| | antigen M6) (OK blood | | | |
| | group antigen) (Tumor | | | |
| | cell-derived collagenase | | | |
| | stimulatory | | | |
| Follistatin-like 1 | | 61.4 | 6.1 | 61435.008 |
| Fractalkine | | 18.3 | 1.8 | 18345.526 |
| Galectin-1 | Galectin-1 (Gal-1) (14 | 49.6-1499.5 | 5.0 | 1499500 |
| | kDa laminin-binding | | | |
| | protein) (HLBP14) (14 | | | |
| | kDa lectin) (Beta- | | | |
| | galactoside-binding | | | |
| | lectin L-14-I) (Galaptin) | | | |
| | (HBL) (HPL) (Lactose- | | | |
| | binding lectin 1) (Lectin | | | |
| | galactoside-binding | | | |
| | soluble 1) (Putative | | | |
| | MAPK-activating protein | | | |
| | PM12) (S-Lac lectin 1) | | | |
| GITR L | | 63.5 | 6.3 | 63489.982 |
| Granulysin | Granulysin (Lymphokine | 37.4-15016.5 | 3.7 | 15016500 |
| | LAG-2) (Protein NKG5) | | | |
| | (T-cell activation protein | | | |
| | 519) | | | |
| IL-1 R3 | Interleukin-1 receptor | 0.5-47.3 | 0.1 | 47300 |
| | accessory protein (IL-1 | | | |
| | receptor accessory | | | |
| | protein) (IL-1RAcP) | | | |
| | (Interleukin-1 receptor 3) | | | |
| | (IL-1R-3) (IL-1R3) | | | |
| IL-15 R | | 2.3 | 0.2 | 2281.6196 |
| IL-17E | | 1.4 | 0.1 | 1424.0357 |
| L1CAM-2 | | 280.3 | 28.0 | 280303.55 |
| LRIG3 | | 46.2 | 4.6 | 46162.447 |
| MEPE | | 2.5 | 0.3 | 2501.2571 |
| Nectin-4 | | 19.7 | 2.0 | 19745.357 |
| Periostin | Periostin (PN) | 0.9-11094.9 | 0.1 | 11094900 |
| | (Osteoblast-specific | | | |
| | factor 2) (OSF-2) | | | |
| Persephin | | 65.8 | 6.6 | 65802.305 |
| Renin | | 2.0-2.4 | 0.2 | 2400 |
| RGM-B | | 19.2 | 1.9 | 19158.505 |
| ROBO3 | | 9.1 | 0.9 | 9057.6684 |
| S100A8 | | 4.9 | 0.5 | 4948.3702 |
| Siglec-7 | | 4.4 | 0.4 | 4398.0532 |
| Syndecan-3 | | 110.1 | 11.0 | 110129.64 |
| Thrombospondin-2 | | 24.6 | 2.5 | 24586.146 |
| Thrombospondin-5 | | 0.6 | 0.1 | 616.61911 |
| Tie-1 | | 26.4 | 2.6 | 26428.685 |
| ULBP-2 | NKG2D ligand 2 (N2DL- | 66.1 | 6.6 | 66122.681 |
| | 2) (NKG2DL2) (ALCAN- | | | |
| | alpha) (Retinoic acid | | | |
| | early transcript 1H) | | | |
| | (UL16-binding protein 2) | | | |
| ANGPTL3 | | 59.8 | 6.0 | 59846.266 |
| bIG-H3 | Transforming growth | 119.6-4221.2 | 12.0 | 4221200 |
| | factor-beta-induced | | | |
| | protein ig-h3 (Beta ig-h3) | | | |
| | (Kerato-epithelin) (RGD- | | | |
| | containing collagen- | | | |
| | associated protein) | | | |
| | (RGD-CAP) | | | |
| CA9 | | 12.0 | 1.2 | 12034.621 |
| Cathepsin B | | 23.2 | 2.3 | 23154.156 |
| CD23 | | 6.5 | 0.7 | 6539.8579 |
| CHI3L1 | | 21.1 | 2.1 | 21118.734 |
| Dkk-4 | | 264.7 | 26.5 | 264677.25 |
| DPPIV | Dipeptidyl peptidase 4 | 486.4-35659.0 | 48.6 | 35659000 |
| | (EC 3.4.14.5) (ADABP) | | | |
| | (Adenosine deaminase | | | |
| | complexing protein 2) | | | |
| | (ADCP-2) (Dipeptidyl | | | |
| | peptidase IV) (DPP IV) | | | |
| | (T-cell activation antigen | | | |
| | CD26) (TP103) (CD | | | |
| | antigen CD26) [Cleaved | | | |
| | into: Dipeptidyl | | | |
| | peptidase 4 membrane | | | |
| | form (Dipeptidyl | | | |
| | peptidase IV membrane | | | |
| | form); Dipeptidyl | | | |
| | peptidase 4 soluble form | | | |
| | (Dipeptidyl peptidase IV | | | |
| | soluble form)] | | | |
| EDA-A2 | Ectodysplasin-A | 144.0 | 14.4 | 144004.74 |
| | (Ectodermal dysplasia | | | |
| | protein) (EDA protein) | | | |
| | [Cleaved into: | | | |
| | Ectodysplasin-A, | | | |
| | membrane form; | | | |
| | Ectodysplasin-A, | | | |
| | secreted form] | | | |
| Epo R | | 33.1 | 3.3 | 33108.103 |
| FGF-6 | | 17.0 | 1.7 | 17005.984 |
| FGF-9 | Fibroblast growth factor | 26.7 | 2.7 | 26713.438 |
| | 9 (FGF-9) (Glia- | | | |
| | activating factor) (GAF) | | | |
| | (Heparin-binding growth | | | |
| | factor 9) (HBGF-9) | | | |
| Gas 1 | | 11.6 | 1.2 | 11557.807 |
| IGFBP-5 | Insulin-like growth | 1,245.9 | 124.6 | 1245948 |
| | factor-binding protein 5 | | | |
| | (IBP-5) (IGF-binding | | | |
| | protein 5) (IGFBP-5) | | | |
| IL-1 F5 | | 6.3 | 0.6 | 6349.2077 |
| IL-1 F6 | | 102.7 | 10.3 | 102653.98 |
| IL-1 F8 | | 1.6 | 0.2 | 1561.463 |
| IL-1 F9 | | 268.1 | 26.8 | 268132.23 |
| IL-1 F10 | | 706.3 | 70.6 | 706256.28 |
| IL-1 R5 | | 15.7 | 1.6 | 15748.91 |
| IL-17C | | 15.8 | 1.6 | 15815.29 |
| IL-18 | | 12.2 | 1.2 | 12203.999 |
| IL-20 | Interleukin-20 (IL-20) | 51-1194.5 | 5.1 | 1194000 |
| | (Cytokine Zcyto10) | | | |
| IL-34 | | 3.5 | 0.4 | 3547.4731 |
| IL-5 Ra | Interleukin-5 receptor | 1,040.2 | 104.0 | 1040223.9 |
| | subunit alpha (IL-5 | | | |
| | receptor subunit alpha) (IL- | | | |
| | 5R subunit alpha) (IL-5R- | | | |
| | alpha) (IL-5RA) (CDw125) | | | |
| | (CD antigen CD125) | | | |
| IL-10 Ra | Interleukin-10 receptor | 5,066.5 | 506.6 | 5066467.2 |
| | subunit alpha (IL-10 | | | |
| | receptor subunit alpha) (IL- | | | |
| | 10R subunit alpha) (IL- | | | |
| | 10RA) (CDw210a) | | | |
| | (Interleukin-10 receptor | | | |
| | subunit 1) (IL-10R subunit | | | |
| | 1) (IL-10R1) (CD antigen | | | |
| | CD210) | | | |
| Layilin | | 14.9 | 1.5 | 14863.552 |
| Leptin R | Leptin receptor (LEP-R) | 693.4 | 69.3 | 693409.21 |
| | (HuB219) (OB receptor) | | | |
| | (OB-R) (CD antigen | | | |
| | CD295) | | | |
| Marapsin | | 25.7 | 2.6 | 25733.96 |
| Mer | Tyrosine-protein kinase | 191.4 | 19.1 | 191389.77 |
| | Mer (EC 2.7.10.1) | | | |
| | (Proto-oncogene c-Mer) | | | |
| | (Receptor tyrosine | | | |
| | kinase MerTK) | | | |
| MMP-7 | | 21.5 | 2.2 | 21509.624 |
| P-Cadherin | | 12.6 | 1.3 | 12621.709 |
| Prostasin | | 32.5 | 3.3 | 32547.02 |
| PSMA | | 2,629.6 | 263.0 | 2629597.7 |
| SIGIRR | Single Ig IL-1-related | 409.4 | 40.9 | 409390.78 |
| | receptor (Single Ig IL- | | | |
| | 1R-related molecule) | | | |
| | (Single immunoglobulin | | | |
| | domain-containing IL1R- | | | |
| | related protein) | | | |
| | (Toll/interleukin-1 | | | |
| | receptor 8) (TIR8) | | | |
| TGFb RIII | Transforming growth | 362.2 | 36.2 | 362176 |
| | factor beta receptor type | | | |
| | 3 (TGF-beta receptor | | | |
| | type 3) (TGFR-3) | | | |
| | (Betaglycan) | | | |
| | (Transforming growth | | | |
| | factor beta receptor III) | | | |
| | (TGF-beta receptor type | | | |
| | III) | | | |
| TF | Tissue factor (TF) | 5.9-88.4 | 0.6 | 88400 |
| | (Coagulation factor III) | | | |
| | (Thromboplastin) (CD | | | |
| | antigen CD142) | | | |
| Albumin | | 239.0-540.9 | 23.9 | 540900 |
| CA19-9 | | 1,511.4 | 151.1 | 1511427.3 |
| CD163 | | 8.4 | 0.8 | 8373.8074 |
| Cystatin C | | 80.6 | 8.1 | 80573.142 |
| Decorin | | 887.1 | 88.7 | 887080.79 |
| Dkk-3 | | 43.1 | 4.3 | 43053.353 |
| Fetuin A | | 225.6-199374.2 | 22.6 | 199374200 |
| FOLR1 | | 57.6 | 5.8 | 57601.162 |
| HAI-2 | | 7.8 | 0.8 | 7764.0163 |
| IL-27 | | 1.4 | 0.1 | 1411.1913 |
| LAG-3 | | 20.6 | 2.1 | 20627.085 |
| RBP4 | | 164.5-5745.2 | 16.5 | 5745200 |
| TACI | | 141.3 | 14.1 | 141318.11 |
| AR | Amphiregulin (AR) | 27.9 | 2.8 | 27899.188 |
| | (Colorectum cell-derived | | | |
| | growth factor) (CRDGF) | | | |
| BDNF | | 0.5 | 0.1 | 480.40447 |
| bFGF | Fibroblast growth factor | 20.1-25.2 | 2.0 | 25200 |
| | 2 (FGF-2) (Basic | | | |
| | fibroblast growth factor) | | | |
| | (bFGF) (Heparin-binding | | | |
| | growth factor 2) (HBGF- | | | |
| | 2) | | | |
| BMP-4 | | 1.7 | 0.2 | 1748.5354 |
| BMP-5 | Bone morphogenetic | 2,007.8 | 200.8 | 2007757.6 |
| | protein 5 (BMP-5) | | | |
| BMP-7 | Bone morphogenetic | 233.5 | 23.3 | 233472.15 |
| | protein 7 (BMP-7) | | | |
| | (Osteogenic protein 1) | | | |
| | (OP-1) (Eptotermin alfa) | | | |
| b-NGF | | 0.1 | 0.1 | 50.686799 |
| EGF | Pro-epidermal growth | 0.1-0.5 | 0.1 | 500 |
| | factor (EGF) [Cleaved into: | | | |
| | Epidermal growth factor | | | |
| | (Urogastrone)] | | | |
| EGF R | Epidermal growth factor | 125.3-187.6 | 12.5 | 187600 |
| | receptor (EC 2.7.10.1) | | | |
| | (Proto-oncogene c-ErbB-1) | | | |
| | (Receptor tyrosine-protein | | | |
| | kinase erbB-1) | | | |
| FGF-4 | | 84.9 | 8.5 | 84904.202 |
| GDF-15 | Growth/differentiation | 0.4-19.7 | 0.1 | 19700 |
| | factor 15 (GDF-15) | | | |
| | (Macrophage inhibitory | | | |
| | cytokine 1) (MIC-1) | | | |
| | (NSAID-activated gene 1 | | | |
| | protein) (NAG-1) | | | |
| | (NSAID-regulated gene | | | |
| | 1 protein) (NRG-1) | | | |
| | (Placental TGF-beta) | | | |
| | (Placental bone | | | |
| | morphogenetic protein) | | | |
| | (Prostate differentiation | | | |
| | factor) | | | |
| GH | | 3.7 | 0.4 | 3732.1841 |
| HB-EGF | | 2.4 | 0.2 | 2360.5215 |
| HGF | Hepatocyte growth | 26.0-81.8 | 2.6 | 81800 |
| | factor (Hepatopoietin-A) | | | |
| | (Scatter factor) (SF) | | | |
| | [Cleaved into: | | | |
| | Hepatocyte growth | | | |
| | factor alpha chain; | | | |
| | Hepatocyte growth | | | |
| | factor beta chain] | | | |
| IGFBP-1 | Insulin-like growth factor- | 1.4-1781.2 | 0.1 | 1781200 |
| | binding protein 1 (IBP-1) | | | |
| | (IGF-binding protein 1) | | | |
| | (IGFBP-1) (Placental | | | |
| | protein 12) (PP12) | | | |
| IGFBP-2 | Insulin-like growth factor- | 25.3-91.3 | 2.5 | 91300 |
| | binding protein 2 (IBP-2) | | | |
| | (IGF-binding protein 2) | | | |
| | (IGFBP-2) | | | |
| IGFBP-3 | Insulin-like growth factor- | 533.0 | 53.3 | 532982.09 |
| | binding protein 3 (IBP-3) | | | |
| | (IGF-binding protein 3) | | | |
| | (IGFBP-3) | | | |
| IGFBP-4 | Insulin-like growth factor- | 1,861.6 | 186.2 | 1861630.9 |
| | binding protein 4 (IBP-4) | | | |
| | (IGF-binding protein 4) | | | |
| | (IGFBP-4) | | | |
| IGFBP-6 | Insulin-like growth factor- | 45.1-183.1 | 4.5 | 183100 |
| | binding protein 6 (IBP-6) | | | |
| | (IGF-binding protein 6) | | | |
| | (IGFBP-6) | | | |
| Insulin | Insulin [Cleaved into: | 10,326.3 | 1,032.6 | 10326254 |
| | Insulin B chain; Insulin A | | | |
| | chain] | | | |
| MCSF R | Macrophage colony- | 393.0 | 39.3 | 393031.19 |
| | stimulating factor 1 | | | |
| | receptor (CSF-1 | | | |
| | receptor) (CSF-1-R) | | | |
| | (CSF-1R) (M-CSF-R) | | | |
| | (EC 2.7.10.1) (Proto- | | | |
| | oncogene c-Fms) (CD | | | |
| | antigen CD115) | | | |
| NGF R | | 1.1 | 0.1 | 1138.7885 |
| NT-3 | | 1.7 | 0.2 | 1683.605 |
| PDGF-AA | | 4.8 | 0.5 | 4770.0148 |
| PlGF | Placenta growth factor | 0.8-10.6 | 0.1 | 10600 |
| | (PlGF) | | | |
| SCF | | 0.8 | 0.1 | 841.7876 |
| SCF R | Mast/stem cell growth | 108.5 | 10.8 | 108461.9 |
| | factor receptor Kit | | | |
| | (SCFR) (EC 2.7.10.1) | | | |
| | (Piebald trait protein) | | | |
| | (PBT) (Proto-oncogene | | | |
| | c-Kit) (Tyrosine-protein | | | |
| | kinase Kit) (p145 c-kit) | | | |
| | (v-kit Hardy-Zuckerman | | | |
| | 4 feline sarcoma viral | | | |
| | oncogene homolog) (CD | | | |
| | antigen CD117) | | | |
| TGFa | Protransforming growth | 28.4 | 2.8 | 28394.755 |
| | factor alpha [Cleaved into: | | | |
| | Transforming growth | | | |
| | factor alpha (TGF-alpha) | | | |
| | (EGF-like TGF) (ETGF) (TGF | | | |
| | type 1)] | | | |
| TGFb1 | Transforming growth | 281.2-3166.1 | 28.1 | 3166100 |
| | factor beta-1 (TGF-beta-1) | | | |
| | [Cleaved into: Latency- | | | |
| | associated peptide (LAP)] | | | |
| TGFb3 | | 61.6 | 6.2 | 61590.355 |
| VEGF | Vascular endothelial | 2.0-109.0 | 0.2 | 109000 |
| | growth factor A (VEGF- | | | |
| | A) (Vascular | | | |
| | permeability factor) | | | |
| | (VPF) | | | |
| VEGF R2 | | 24.4 | 2.4 | 24401.273 |
| BCMA | Tumor necrosis factor | 261.3 | 26.1 | 261322.53 |
| | receptor superfamily | | | |
| | member 17 (B-cell | | | |
| | maturation protein) (CD | | | |
| | antigen CD269) | | | |
| CEACAM-1 | Carcinoembryonic | 88.7 | 8.9 | 88745.144 |
| | antigen-related cell | | | |
| | adhesion molecule 1 | | | |
| | (Biliary glycoprotein 1) | | | |
| | (BGP-1) (CD antigen | | | |
| | CD66a) | | | |
| E-Selectin | | 17.9 | 1.8 | 17903.141 |
| Fas | Tumor necrosis factor | 6.3 | 0.6 | 6328.2205 |
| | receptor superfamily | | | |
| | member 6 (Apo-1 | | | |
| | antigen) (Apoptosis- | | | |
| | mediating surface | | | |
| | antigen FAS) (FASLG | | | |
| | receptor) (CD antigen | | | |
| | CD95) | | | |
| Flt-3L | | 1.7 | 0.2 | 1690.4949 |
| HVEM | | 7.6 | 0.8 | 7586.0775 |
| IL-2 Rg | | 24.2 | 2.4 | 24170.021 |
| IL-10 Rb | | 0.5 | 0.1 | 507.00864 |
| IL-17R | | 2.0 | 0.2 | 2040.8122 |
| IL-21R | | 5.5 | 0.6 | 5513.407 |
| LIMPII | Lysosome membrane | 689.9 | 69.0 | 689881.16 |
| | protein 2 (85 kDa | | | |
| | lysosomal membrane | | | |
| | sialoglycoprotein) (LGP85) | | | |
| | (CD36 antigen-like 2) | | | |
| | (Lysosome membrane | | | |
| | protein II) (LIMP II) | | | |
| | (Scavenger receptor class | | | |
| | B member 2) (CD antigen | | | |
| | CD36) | | | |
| Lipocalin-2 | Neutrophil gelatinase- | 27.7-390.9 | 2.8 | 390300 |
| | associated lipocalin (NGAL) | | | |
| | (25 kDa alpha-2- | | | |
| | microglobulin-related | | | |
| | subunit of MMP-9) | | | |
| | (Lipocalin-2) (Oncogene | | | |
| | 24p3) (Siderocalin LCN2) | | | |
| | (p25) | | | |
| LYVE-1 | Lymphatic vessel | 185.3 | 18.5 | 185333.66 |
| | endothelial hyaluronic | | | |
| | acid receptor 1 (LYVE-1) | | | |
| | (Cell surface retention | | | |
| | sequence-binding | | | |
| | protein 1) (CRSBP-1) | | | |
| | (Extracellular link | | | |
| | domain-containing | | | |
| | protein 1) (Hyaluronic | | | |
| | acid receptor) | | | |
| NRG1-b1 | | 0.6 | 0.1 | 643.53448 |
| PECAM-1 | | 6.0 | 0.6 | 6049.5214 |
| TRAIL R3 | | 1.8 | 0.2 | 1773.4435 |
| Trappin-2 | | 1.0-28.9 | 0.1 | 28900 |
| uPAR | Urokinase plasminogen | 6.2-191.0 | 0.6 | 191000 |
| | activator surface | | | |
| | receptor (U-PAR) | | | |
| | (uPAR) (Monocyte | | | |
| | activation antigen Mo3) | | | |
| | (CD antigen CD87) | | | |
| XEDAR | | 2.0 | 0.2 | 2036.9134 |

As described herein, in embodiments, the bio-mimetic formulations include at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, etc. of the recombinant peptides listed in Table 9 above. In further embodiments, the bio-mimetic formulations include all of the recombinant peptides, or between 1-280 of the recombinant peptides, or between 10-280, between 50-280, between 100-280, between 150-280, between 200-280, between about 50-200, between about 5-100, etc., of the recombinant peptides listed above in Table 9.

The amounts of the recombinant peptides listed above in Table 9 represent an exemplary amount, as well as a suitably lower and upper bound for the amounts of the recombinant peptides. In addition, one or more of the recombinant peptides in the bio-mimetic formulations (including those from Table 9) can be increased or reduced relative to one or more other recombinant peptides in the bio-mimetic formulations by about 1-500%, about 1-200%, about 1-100%, about 1-90%, about 1-80%, about 1-70%, about 1-60%, about 1-50%, about 1-40%, about 1-30%, about 1-20%, about 1-10%, about 1-5%, about 10%-20%, about 20%-30%, about 30%-40%, about 40%-50%, about 50%-60%, about 60%-70%, about 70%-80%, about 80%-90%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, etc. In addition, the amounts of various recombinant peptides can be increased by the amounts noted, while the amounts of other recombinant peptides are reduced. In further embodiments, the amounts of the various recombinant peptides, including those listed in Table 9, can be increased or reduced by 0.1-times, 2-times, 5-times, 10-times, 100-times, or even 1000-times above and below those exemplary amounts and exemplary ranges provided herein.

For example, in still further embodiments, the amounts of the recombinant peptides from Table 9 can be adjusted by about 0.5 times below to about 50 times above the exemplary amounts, as set forth below in Table 10. Additional amounts above and below these ranges, as described herein, can also be utilized in the formulations.

TABLE 10

| | |
|---|---|
| Recombinant Protein | About (pg/mL) to About (pg/mL) |

| | | |
|---|---|---|
| ENA-78 | 0.804176 | 80.41759 |
| GRO | 0.3 | 85 |
| IL-18 BPa | 2.040756 | 204.0756 |
| IL-31 | 0.098423 | 9.842347 |
| I-TAC | 2.11694 | 211.694 |
| LIF | 1.689267 | 168.9267 |
| LIGHT | 1.250465 | 125.0465 |
| Lymphotactin | 11.96601 | 1196.601 |
| MCP-2 | 0.275985 | 27.5985 |
| MCP-3 | 0.304568 | 30.45679 |
| MCP-4 | 0.14624 | 14.62395 |
| MIP-3a | 0.114982 | 11.49817 |
| MIP-3b | 0.55 | 175 |
| MPIF-1 | 0.199996 | 19.99956 |
| MSP | 10.47916 | 1047.916 |
| NAP-2 | 0.344567 | 34.45667 |
| OPN | 0.378206 | 37.82061 |
| PARC | 0.3 | 365 |
| PF4 | 0.1 | 21670 |
| SDF-1a | 0.781459 | 78.14588 |
| TECK | 4.720171 | 472.0171 |
| ADAM9 | 199.8912 | 19989.12 |
| Cathepsin L | 25.37929 | 2537.929 |
| CD97 | 828.7042 | 82870.42 |
| FAP | 4.289186 | 428.9186 |
| Galectin-3 | 186.329 | 18632.9 |
| IGF-2R | 2992.449 | 299244.9 |
| Neprilysin | 416.0709 | 41607.09 |
| NOV | 1.22945 | 122.945 |
| PGRP-S | 4.679867 | 467.9867 |
| Serpin A4 | 2320.416 | 232041.6 |
| TLR2 | 61.01992 | 6101.992 |
| Transferrin | 2312.85 | 811945 |
| Albumin | 119.5 | 27045 |
| CA19-9 | 755.7136 | 75571.36 |
| CD163 | 4.186904 | 418.6904 |
| Cystatin C | 40.28657 | 4028.657 |
| Decorin | 443.5404 | 44354.04 |
| Dkk-3 | 21.52668 | 2152.668 |
| Fetuin A | 112.8 | 9968700 |
| FOLR1 | 28.80058 | 2880.058 |
| HAI-2 | 3.882008 | 388.2008 |
| IL-27 | 0.705596 | 70.55956 |
| LAG-3 | 10.31354 | 1031.354 |
| RBP4 | 827.25 | 287260 |
| TACI | 70.65906 | 7065.906 |
| Adiponectin | 122.2949 | 12229.49 |
| ANGPTL4 | 16.48101 | 1648.101 |
| B2M | 34.3 | 26005 |
| BCAM | 9.615331 | 961.5331 |
| CA125 | 54.4 | 690740 |
| CEA | 51.24861 | 5124.861 |
| CRP | 5.312363 | 531.2363 |
| Ferritin | 317.9582 | 31795.82 |
| FSH | 3.568339 | 356.8339 |
| hCGb | 11.27181 | 1127.181 |
| IL-3 | 7.801464 | 780.1464 |
| IL-21 | 162.2792 | 16227.92 |
| Leptin | 4.90557 | 490.557 |
| MMP-1 | 19.13294 | 1913.294 |
| MMP-2 | 246.7212 | 24672.12 |
| MMP-3 | 8.442105 | 844.2105 |
| MMP-8 | 7.35 | 945 |
| MMP-9 | 16.58751 | 1658.751 |
| MMP-10 | 0.25 | 65 |
| MMP-13 | 4.95 | 2995 |
| NCAM-1 | 37.3642 | 3736.42 |
| Nidogen-1 | 5.25 | 533185 |
| NSE | 8.10235 | 810.235 |
| OSM | 9.794592 | 979.4592 |
| Prolactin | 222.1673 | 22216.73 |
| PSA-free | 8.466777 | 846.6777 |
| Siglec-9 | 9.38912 | 938.912 |
| TACE | 8.85 | 8740 |
| TIMP-4 | 2.955022 | 295.5022 |
| Activin A | 183.3023 | 18330.23 |
| AgRP | 0.617739 | 61.77394 |
| Angiogenin | 2.3 | 3220 |
| ANG-1 | 5.046102 | 504.6102 |
| Angiostatin | 206.65 | 268810 |
| Cathepsin S | 4.8 | 520 |
| CD40 | 7.483767 | 748.3767 |
| Cripto-1 | 2.487562 | 248.7562 |
| DAN | 52.53055 | 5253.055 |
| DKK-1 | 13.5641 | 1356.41 |
| E-Cadherin | 70.83771 | 7083.771 |
| EpCAM | 1.599139 | 159.9139 |
| FAS L | 15.3679 | 1536.79 |
| Fcg RIIBC | 40.57073 | 4057.073 |
| Follistatin | 0.9 | 625 |
| Galectin-7 | 19.4 | 14620 |
| ICAM-2 | 74.65406 | 7465.406 |
| IL-13 R1 | 0.7 | 315 |
| IL-13 R2 | 23.7 | 10450 |
| IL-17B | 2.603124 | 260.3124 |
| IL-2 Ra | 3.020166 | 302.0166 |
| IL-23 | 1.35 | 1740 |
| LAP(TGFb1) | 0.1 | 2635 |
| NrCAM | 15.68772 | 1568.772 |
| PAI-1 | 0.3 | 105165 |
| PDGF-AB | 6.300926 | 630.0926 |
| Resistin | 14.7975 | 1479.75 |
| SDF-1b | 1.844113 | 184.4113 |
| gp130 | 78.6236 | 7862.36 |
| Shh-N | 11.95312 | 1195.312 |
| Siglec-5 | 11.96323 | 1196.323 |
| IL-1 R4 | 3.553903 | 355.3903 |
| TGFb2 | 13.77383 | 1377.383 |
| Tie-2 | 38.4099 | 3840.99 |
| TPO | 33.70269 | 3370.269 |
| TRAIL R4 | 28.90779 | 2890.779 |
| TREM-1 | 11.98147 | 1198.147 |
| VEGF-C | 5.835122 | 583.5122 |
| VEGF R1 | 29.7 | 22235 |
| ADAM8 | 2.96864 | 296.864 |
| B7-H3 | 35.45 | 5370 |
| Cadherin-13 | 5.475914 | 547.5914 |
| CD48 | 116.669 | 11666.9 |
| CD99 | 1.65 | 4185 |
| CD229 | 21.16579 | 2116.579 |
| CEACAM-5 | 103.5 | 18335 |
| Cystatin A | 0.7 | 125 |
| Cystatin B | 2.9 | 3865 |
| Cystatin E M | 1.3 | 12395 |
| Desmoglein 2 | 0.25 | 640 |
| DR3 | 106.0861 | 10608.61 |
| ErbB4 | 0.535154 | 53.51535 |
| ESAM | 1.367835 | 136.7835 |
| FGF-21 | 3.412462 | 341.2462 |
| Galectin-2 | 59.57017 | 5957.017 |
| Galectin-9 | 9.4 | 18380 |
| JAM-B | 11.05264 | 1105.264 |
| Kallikrein 5 | 64.69401 | 6469.401 |
| Midkine | 9.9 | 1600 |
| Pentraxin 3 | 15.7 | 5045 |
| Pref-1 | 277.4122 | 27741.22 |
| Siglec-10 | 133.3905 | 13339.05 |
| SLAM | 69.08294 | 6908.294 |
| SP-D | 54.84277 | 5484.277 |
| Syndecan-4 | 1.7 | 3895 |
| Testican 2 | 15.11966 | 1511.966 |
| TIM-3 | 12.72207 | 1272.207 |
| TLR4 | 5.864784 | 586.4784 |
| TRAIL | 2.095214 | 209.5214 |
| ULBP-1 | 27.17043 | 2717.043 |
| ADAMTS13 | 19.34619 | 1934.619 |
| Aggrecan | 0.210963 | 21.09632 |
| Angiotensinogen | 16.35 | 18430 |
| B7-H1 | 34.89095 | 3489.095 |
| BMPR-IA | 46.01177 | 4601.177 |
| BMPR-II | 54.52558 | 5452.558 |
| Cadherin-11 | 1.8 | 225 |
| CD27 | 2.006599 | 200.6599 |
| Ck beta 8-1 | 0.762111 | 76.21114 |
| EMMPRIN | 10.5 | 89825 |
| Follistatin-like 1 | 30.7175 | 3071.75 |
| Fractalkine | 9.172763 | 917.2763 |
| Galectin-1 | 24.8 | 74975 |
| GITR L | 31.74499 | 3174.499 |
| Granulysin | 18.7 | 750825 |
| IL-1 R3 | 0.25 | 2365 |
| IL-15 R | 1.14081 | 114.081 |
| IL-17E | 0.712018 | 71.20179 |
| L1CAM-2 | 140.1518 | 14015.18 |
| LRIG3 | 23.08122 | 2308.122 |
| MEPE | 1.250629 | 125.0629 |
| Nectin-4 | 9.872678 | 987.2678 |
| Periostin | 0.45 | 554745 |
| Persephin | 32.90115 | 3290.115 |
| Renin | 1 | 120 |
| RGM-B | 9.579253 | 957.9253 |
| ROBO3 | 4.528834 | 452.8834 |
| S100A8 | 2.474185 | 247.4185 |
| Siglec-7 | 2.199027 | 219.9027 |
| Syndecan-3 | 55.06482 | 5506.482 |
| Thrombospondin-2 | 12.29307 | 1229.307 |
| Thrombospondin-5 | 0.30831 | 30.83096 |
| Tie-1 | 13.21434 | 1321.434 |
| ULBP-2 | 33.06134 | 3306.134 |
| ANGPTL3 | 29.92313 | 2992.313 |
| bIG-H3 | 59.8 | 211060 |
| CA9 | 6.017311 | 601.7311 |
| Cathepsin B | 11.57708 | 1157.708 |
| CD23 | 3.269929 | 326.9929 |
| CHI3L1 | 10.55937 | 1055.937 |
| Dkk-4 | 132.3386 | 13233.86 |
| DPPIV | 243.2 | 1782950 |
| EDA-A2 | 72.00237 | 7200.237 |
| Epo R | 16.55405 | 1655.405 |
| FGF-6 | 8.502992 | 850.2992 |
| FGF-9 | 13.35672 | 1335.672 |
| Gas 1 | 5.778903 | 577.8903 |
| IGFBP-5 | 622.974 | 62297.4 |
| IL-1 F5 | 3.174604 | 317.4604 |
| IL-1 F6 | 51.32699 | 5132.699 |
| IL-1 F8 | 0.780731 | 78.07315 |
| IL-1 F9 | 134.0661 | 13406.61 |
| IL-1 F10 | 353.1281 | 35312.81 |
| IL-1 R5 | 7.874455 | 787.4455 |
| IL-17C | 7.907645 | 790.7645 |
| IL-18 | 6.101999 | 610.1999 |
| IL-20 | 25.5 | 59725 |
| IL-34 | 1.773737 | 177.3737 |
| IL-5 Ra | 520.112 | 52011.2 |
| IL-10 Ra | 2533.234 | 253323.4 |
| Layilin | 7.431776 | 743.1776 |
| Leptin R | 346.7046 | 34670.46 |
| Marapsin | 12.86698 | 1286.698 |
| Mer | 95.69488 | 9569.488 |
| MMP-7 | 10.75481 | 1075.481 |
| P-Cadherin | 6.310855 | 631.0855 |
| Prostasin | 16.27351 | 1627.351 |
| PSMA | 1314.799 | 131479.9 |
| SIGIRR | 204.6954 | 20469.54 |
| TGFb RIII | 181.088 | 18108.8 |
| TF | 2.95 | 4420 |
| Albumin | 119.5 | 27045 |
| CA19-9 | 755.7136 | 75571.36 |
| CD163 | 4.186904 | 418.6904 |
| Cystatin C | 40.28657 | 4028.657 |
| Decorin | 443.5404 | 44354.04 |
| Dkk-3 | 21.52668 | 2152.668 |
| Fetuin A | 112.8 | 9968710 |
| FOLR1 | 28.80058 | 2880.058 |
| HAI-2 | 3.882008 | 388.2008 |
| IL-27 | 0.705596 | 70.55956 |
| LAG-3 | 10.31354 | 1031.354 |
| RBP4 | 82.25 | 287260 |
| TACI | 70.65906 | 7065.906 |
| AR | 13.94959 | 1394.959 |
| BDNF | 0.240202 | 24.02022 |
| bFGF | 10.05 | 1260 |
| BMP-4 | 0.874268 | 87.42677 |
| BMP-5 | 1003.879 | 100387.9 |
| BMP-7 | 116.7361 | 11673.61 |
| b-NGF | 0.025343 | 2.53434 |
| EGF | 0.1 | 25 |
| EGF R | 62.65 | 9380 |
| FGF-4 | 42.4521 | 4245.21 |
| GDF-15 | 0.2 | 985 |
| GH | 1.866092 | 186.6092 |
| HB-EGF | 1.180261 | 118.0261 |
| HGF | 13 | 4090 |
| IGFBP-1 | 0.7 | 89060 |
| IGFBP-2 | 12.65 | 4565 |
| IGFBP-3 | 266.491 | 26649.1 |
| IGFBP-4 | 930.8155 | 93081.55 |
| IGFBP-6 | 22.55 | 9155 |
| Insulin | 5163.127 | 516312.7 |
| MCSF R | 196.5156 | 19651.56 |
| NGF R | 0.569394 | 56.93942 |
| NT-3 | 0.841803 | 84.18025 |
| PDGF-AA | 2.385007 | 238.5007 |
| PlGF | 0.4 | 530 |
| SCF | 0.420894 | 42.08938 |
| SCF R | 54.23095 | 5423.095 |
| TGFa | 14.19738 | 1419.738 |
| TGFb1 | 140.6 | 158305 |
| TGFb3 | 30.79518 | 3079.518 |
| VEGF | 1 | 5450 |
| VEGF R2 | 12.20064 | 1220.064 |
| BCMA | 130.6613 | 13066.13 |
| CEACAM-1 | 44.37257 | 4437.257 |
| E-Selectin | 8.95157 | 895.157 |
| Fas | 3.16411 | 316.411 |
| Flt-3L | 0.845247 | 84.52475 |
| HVEM | 3.793039 | 379.3039 |
| IL-2 Rg | 12.08501 | 1208.501 |
| IL-10 Rb | 0.253504 | 25.35043 |
| IL-17R | 1.020406 | 102.0406 |
| IL-21R | 2.756703 | 275.6703 |
| LIMPII | 344.9406 | 34494.06 |
| Lipocalin-2 | 13.85 | 19545 |
| LYVE-1 | 92.66683 | 9266.683 |
| NRG1-b1 | 0.321767 | 32.17672 |
| PECAM-1 | 3.024761 | 302.4761 |
| TRAIL R3 | 0.886722 | 88.67218 |
| Trappin-2 | 0.5 | 1445 |
| uPAR | 3.1 | 9550 |
| XEDAR | 1.018457 | 101.8457 |

Also provided herein are methods of treating a condition, illness, disease, etc., in mammal, suitably a human. In embodiments, the bio-mimetic formulations described herein are applied to a mammal, for example in the form of a liquid or as a solid membrane, etc. In further embodiments, provided herein are methods of preventing a condition, illness, disease, etc., in a mammal, suitably a human. Such embodiments include applying the formulations to a mammal or administering the formulations to a mammal, for example as a liquid, solid, or other suitably dosage form. The formulations can also be used in methods of improving the overall wellness of a mammal, i.e., rather than treating or preventing a particular disease, illness or condition, the formulations can be administered to simply increases the wellness or health state of a mammal.

In embodiments, also provided herein are methods of administering the formualtions described herein to a mammal, such as for example by a medical professional. Such methods of administration can be applying a liquid or solid dosage form to a skin surface of a mammal, or injecting a liquid dosage form, via intravenous, subcutaneous, or intramuscular injection. Exemplary solid dosage forms which can be administered include various capsule or tablet forms, including the use of non-active ingredients such as buffers, stabilizers, binding agents, lubricants, etc.

In exemplary embodiments, methods of treating a wound in a mammal, e.g., a human, are provided which include applying one of the various bio-mimetic formulations described herein, to the wound. Exemplary wounds that can be treated using the methods provided herein include an ulcer, a burn, an abrasion or a laceration. The bio-mimetic formulations can be in the form a liquid, and the bio-mimetic formulations covered with a dressing (e.g., bandage, gauze, etc.), or the bio-mimetic formulation can be in the form of a graft or wound dressing as a solid carrier, and can be applied directly to the wound.

In further embodiments, the various bio-mimetic formulations described herein can be utilized for treatment of pathologies in which connective tissue regeneration, rejuvenation or remodeling is involved, including for example, hair regrowth, wrinkle treatment and elimination, lysis of abdominal and pelvic adhesions, transformation of dsytrophic collagen back to native state (reset piezoelectric tensegrity), i.e., remodeling scar tissue, intrafascial drug delivery, tensegrity face lift, tensegrity soft tissue restoration (neck, hands, knees, chest), transformation of grey hair back to original color, resolved heterotopic bone, breast implant capsule-plasty (reduced scar tissue/capsule constricting implant), migraines, tinnitus, etc.

The various bio-mimetic formulations can be applied to a mammal in need of treatment, e.g., a wound treatment, on a repeated basis. For example, the bio-mimetic formulations can be applied daily over a period of at least two weeks to facilitate treatment of a condition, including for example, the healing of a wound, or any of the various pathologies described herein.

### EXAMPLES

### Example 1

### Wound Healing Scratch Assay

A bio-mimeitc formulation including a liquid carrier and the recombinant peptides set forth below is prepared by mixing the recombinant peptides in the liquid carrier to form a mixture. The liquid carrier includes water, and can include one or more buffers, one or more stabilizing agents and one or more preservatives. The recombinant peptides include:

### Recombinant Proteins for Bio-Mimetic Formulation

at least one of:
   Basic fibroblast growth factor (bFGF) at about 4440 (pg/mL) to about 44400 (pg/mL); or
   Epidermal growth factor (EGF) at about 16.4 (pg/mL) to about 164 (pg/mL); or
   Granulocyte colony-stimulating factor (GCSF) at about 144 (pg/mL) to about 1440 (pg/mL); and
at least one of:
   Platelet-derived growth factor (PDGF-AA) at about 34327 (pg/mL) to about 343270 (pg/mL); or
   Platelet-derived growth factor (PDGF-BB) at about 106 (pg/mL) to about 1060 (pg/mL); or
   Placental growth factor (PLGF) at about 370 (pg/mL) to about 3700 (pg/mL); and
at least one of:
   Transforming growth factor alpha (TGF)-alpha at about 3.4 (pg/mL) to about 34 (pg/mL); or
   Transforming growth factor beta 1 (TGF-B1) at about 1180 (pg/mL) to about 11800 (pg/mL); and
at least one of:
   interleukin 4 (IL-4) at about 2.2 (pg/mL) to about 22 (pg/mL); or
   interleukin 6 (IL-6) at about 74 (pg/mL) to about 740 (pg/mL); or
   interleukin 8 (IL-8) at about 2875 (pg/mL) to about 28750 (pg/mL); or
   interleukin 10 (IL-10) at about 4.1 (pg/mL) to about 41 (pg/mL); and
at least one of:
   Tissue inhibitor of metalloproteinase (TIMP-1) at about 16630 (pg/mL) to about 166300 (pg/mL); or
   Tissue inhibitor of metalloproteinase (TIMP-2) at about 2960 (pg/mL) to about 29600 (pg/mL); or
   Tissue inhibitor of metalloproteinase (TIMP-4) at about 111 (pg/mL) to about 1110 (pg/mL); and
at least one of:
   Growth Differentiation Factor (GDF-15) at about 81.25 (pg/mL) to about 812.5 (pg/mL); or
   Granulocyte macrophage colony-stimulating factor (GM-CSF) at about 0.21 (pg/mL) to about 2.1 (pg/mL); and
Interferon gamma (IFNγ) at about 2.75 (pg/mL) to about 27.5 (pg/mL); and
at least one of:
   Interleukin 1 alpha (IL1-alpha) at about 12.5 (pg/mL) to about 125 (pg/mL); or
   Interleukin 1 Beta (IFN-β) at about 27.8 (pg/mL) to about 278 (pg/mL); or
   Interleukin 1 receptor antagonist (IL-1ra) at about 78.5 (pg/mL) to about 785 (pg/mL); or
   Interleukin 5 (IL-5) at about 2.88 (pg/mL) to about 28.8 (pg/mL); or
   Interleukin 7 (IL-7) at about 1.37 (pg/mL) to about 13.7 (pg/mL); or
   Interleukin 12 p40 (IL-12p40) at about 9.55 (pg/mL) to about 95.5 (pg/mL); or
   Interleukin 12 p70 (IL-12p70) at about 0.79 (pg/mL) to about 7.9 (pg/mL); or
   Interleukin 15 (IL-15) at about 1.35 (pg/mL) to about 13.5 (pg/mL); or
   Interleukin 17 (IL-17) at about 1.1 (pg/mL) to about 11 (pg/mL); or
   Interleukin 16 (IL-16) at about 34.4 (pg/mL) to about 344 (pg/mL); and
Macrophage colony-stimulating factor (MCSF) at about 4.3 (pg/mL) to about 43 (pg/mL); and
Osteoprotegerin (OPG) at about 319.69 (pg/mL) to about 3196.9 (pg/mL); and
B lymphocyte chemoattractant (CXCL13) (BLC) at about 60 (pg/mL) to about 600 (pg/mL); and
Chemokine ligand 1 (CCL1) (1-309) at about 4 (pg/mL) to about 40 (pg/mL); and
Eotaxin-2 at about 0.13 (pg/mL) to about 1.3 (pg/mL); and
Monocyte chemotactic protein 1 (CCL2) (MCP-1) at about 76.95 (pg/mL) to about 769.5 (pg/mL); and
Monokine induced by gamma interferon (CXCL9) (MIG) at about 780 (pg/mL) to about 7800 (pg/mL); and
at least one of:
   Macrophage inflammatory protein 1 alpha (CCL3) (MIP-1α) at about 13.08 (pg/mL) to about 130.8 (pg/mL); or
   Macrophage inflammatory protein 1 beta (CCL4) (MIP-1β) at about 6.56 (pg/mL) to about 65.6 (pg/mL); or
   Macrophage inflammatory protein 1D (MIP-5, CCL15) (MIP-1d) at about 4.3 (pg/mL) to about 43 (pg/mL); and
Regulated on activation, normal T cell expressed and secreted (CCLS) (RANTES) at about 203 (pg/mL) to about 2030 (pg/mL); and
Brain-derived neurotrophic factor (BDNF) at about 45.03 (pg/mL) to about 450.3 (pg/mL); and
Bone morphogenetic protein 5 (BMP-5) at about 90.55 (pg/mL) to about 905.5 (pg/mL); and
Endocrine gland-derived vascular endothelial growth factor (EG-VEGF) at about 496.68 (pg/mL) to about 4966.8 (pg/mL); and
Fibroblast growth factor 4 (FGF-4) at about 353.37 (pg/mL) to about 3533.7 (pg/mL); and
Keratinocyte growth factor (FGF-7) at about 46.7 (pg/mL) to about 467 (pg/mL); and
Growth hormone (GH) at about 114.23 (pg/mL) to about 1142.3 (pg/mL); and
at least one of:
   Insulin-like growth factor (IGF-I) at about 27.65 (pg/mL) to about 276.5 (pg/mL); or
   Insulin-like growth factor binding protein-1 (IGFBP-1) at about 353.51 (pg/mL) to about 3535.1 (pg/mL); or
   Insulin-like growth factor binding protein-2 (IGFBP-2) at about 1072.52 (pg/mL) to about 10725.2 (pg/mL); or
   Insulin-like growth factor binding protein-3 (IGFBP-3) at about 7701.19 (pg/mL) to about 77011.9 (pg/mL); or
   Insulin-like growth factor binding protein-4 (IGFBP-4) at about 2954.34 (pg/mL) to about 29543.4 (pg/mL); or
   Insulin-like growth factor binding protein-6 (IGFBP-6) at about 5162.16 (pg/mL) to about 51621.6 (pg/mL)

In vitro experiments are carried out to determine the effect of the bio-mimetic formulation on human fibroblast and human keratinocyte migration (independently) in a Wound Healing Scratch Assay. For this assay, human keratinocyte cells are seeded to form a confluent monolayer on Day 0. After verifying existence of a monolayer, on Day 1 a scratch is made to ‘wound’ the cellular monolayer. Microscopy is used to follow the migration of the cells as the monolayer is re-established. The microscopy data is analyzed to determine cellular migratory speed as well as other important parameters.

The results are expected to demonstrate improved cellular vitality and migration in combination with the bio-mimemitic formulation, as compared to controls.

### Example 2

### Topical Application of Bio-mimeitc Formulation

The bio-mimetic formulation of Example 1 is prepared for topical application onto the skin of a mammalian patient, for example a human.

The bio-memtic formulation is applied to a wound on the skin surface of the mammalian patient twice per day, for a period of at least 4 weeks.

Following the treatment, the wound on the skin surface of the mammal is found to heal more rapidly than a control wound (i.e., untreated, or treated only with a liquid carrier composition).

### Example 3

### Injection of Bio-mimeitc Formulation

The bio-mimetic formulation of Example 1 is prepared for administration via injection into a mammalian patient.

The bio-memtic formulation is injected into an area of the mammal where connective tissue rejuvenation is desired.

Following the treatment, the connective tissue is found to rejuvinate more rapidly than a control tissue (i.e., untreated, or treated only with a liquid carrier composition).

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of treating a connective tissue, comprising:
administering via injection to the connective tissue a formulation, comprising:
a. a carrier;
b. the following 18 recombinant peptides at the specified amounts:
Basic fibroblast growth factor (bFGF) at about 4440 (pg/mL) to about 44400 (pg/mL);
Platelet-derived growth factor (PDGF-AA) at about 34327 (pg/mL) to about 343270 (pg/mL);
Platelet-derived growth factor (PDGF-BB) at about 106 (pg/mL) to about 1060 (pg/mL);
Transforming growth factor alpha (TGF-alpha) at about 3.4 (pg/mL) to about 34 (pg/mL);
Transforming growth factor beta 1 (TGF-β1) at about 3540 (pg/mL) to about 11800 (pg/mL);
Tissue inhibitor of metalloproteinase (TIMP-1) at about 16630 (pg/mL) to about 166300 (pg/mL);
Growth Differentiation Factor (GDF-15) at about 81.25 (pg/mL) to about 812.5 (pg/mL);
Interferon gamma (IFNγ) at about 2.75 (pg/mL) to about 27.5 (pg/mL);
Macrophage colony-stimulating factor (MCSF) at about 4.3 (pg/mL) to about 43 (pg/mL);
Osteoprotegerin (OPG) at about 319.69 (pg/mL) to about 3196.9 (pg/mL);
B lymphocyte chemoattractant (CXCL13) (BLC) at about 60 (pg/mL) to about 600 (pg/mL);
Chemokine ligand 1 (CCL1) (1-309) at about 4 (pg/mL) to about 40 (pg/mL);
Eotaxin-2 at about 0.13 (pg/mL) to about 1.3 (pg/mL);
Monokine induced by gamma interferon (CXCL9) (MIG) at about 780 (pg/mL) to about 7800 (pg/mL);
Regulated on activation, normal T cell expressed and secreted (CCL5) (RANTES) at about 203 (pg/mL) to about 2030 (pg/mL);
Bone morphogenetic protein 5 (BMP-5) at about 90.55 (pg/mL) to about 905.5 (pg/mL);
Endocrine gland-derived vascular endothelial growth factor (EG-VEGF) at about 496.68 (pg/mL) to about 4966.8 (pg/mL);
Fibroblast growth factor 4 (FGF-4) at about 353.37 (pg/mL) to about 3533.7 (pg/mL); and
c. at least 5 of the following additional recombinant peptides:
Epidermal growth factor (EGF);
Granulocyte colony-stimulating factor (GCSF);
Platelet-derived growth factor (PDGF-AA);
Platelet-derived growth factor (PDGF-BB);
Placental growth factor (PLGF);
Transforming growth factor beta 1 (TGF-B1);
Interleukin 6 (IL-6);
Interleukin 8 (IL-8);
Interleukin 10 (IL-10);
Tissue inhibitor of metalloproteinase (TIMP-2);
Tissue inhibitor of metalloproteinase (TIMP-4);
Monocyte chemotactic protein 1 (CCL2) (MCP-1);
Macrophage inflammatory protein 1 alpha (CCL3) (MIP-1α);
Granulocyte macrophage colony-stimulating factor (GM-CSF);
Interleukin 1 alpha (IL-1alpha)
Interleukin 1 Beta (IFNβ);
Interleukin 1 receptor antagonist (IL-1ra);
Interleukin 5 (IL-5);
Interleukin 7 (IL-7);
Interleukin 12 p40 (IL-12p40);
Interleukin 12 p70 (IL-12p70);
Interleukin 15 (IL-15);
Interleukin 17 (IL-17);
Interleukin 16 (IL-16);
Macrophage inflammatory protein 1 alpha (CCL3) (MIP-α);
Macrophage inflammatory protein 1 beta (CCL4) (MIP-1β);
Macrophage inflammatory protein 1D (MIP-5, CCL15) (MIP-1d);
Keratinocyte growth factor (FGF-7);
Growth hormone (GH);
Osteoprotegerin (OPG);
Brain-derived neurotrophic factor (BDNF));
Insulin-like growth factor binding protein-1 (IGFBP-1);
Insulin-like growth factor binding protein-2 (IGFBP-2);
Insulin-like growth factor binding protein-3 (IGFBP-3);
Insulin-like growth factor binding protein-4 (IGFBP-4); and
Insulin-like growth factor binding protein-6 (IGFBP-6),
thereby regenerating, rejuvenating or remodeling the connective tissue.

2. The method of claim 1, wherein the formulation comprises at least 30 of the additional recombinant peptides.

3. The method of claim 1, wherein the formulation comprises each of the additional recombinant peptides.

4. The method of claim 1, wherein the carrier is a liquid carrier comprising one or more of a buffer, a salt, water and serum.

5. A method of treating a connective tissue, comprising:
administering via injection to the connective tissue a formulation, comprising:
a. a carrier; and
b. the following recombinant peptides:
at least one of:
Basic fibroblast growth factor (bFGF) at about 4440 (pg/mL) to about 44400 (pg/mL); or
Epidermal growth factor (EGF) at about 16.4 (pg/mL) to about 164 (pg/mL); or
Granulocyte colony-stimulating factor (GCSF) at about 144 (pg/mL) to about 1440 (pg/mL); and
at least one of:
Platelet-derived growth factor (PDGF-AA) at about 34327 (pg/mL) to about 343270 (pg/mL); or
Platelet-derived growth factor (PDGF-BB) at about 106 (pg/mL) to about 1060 (pg/mL); or
Placental growth factor (PLGF) at about 370 (pg/mL) to about 3700 (pg/mL); and
at least one of:
Transforming growth factor alpha (TGF)-alpha at about 3.4 (pg/mL) to about 34 (pg/mL); or
Transforming growth factor beta 1 (TGF-B1) at about 1180 (pg/mL) to about 11800 (pg/mL); and
at least one of:
interleukin 4 (IL-4) at about 2.2 (pg/mL) to about 22 (pg/mL); or
interleukin 6 (IL-6) at about 74 (pg/mL) to about 740 (pg/mL); or
interleukin 8 (IL-8) at about 2875 (pg/mL) to about 28750 (pg/mL); or
interleukin 10 (IL-10) at about 4.1 (pg/mL) to about 41 (pg/mL); and
at least one of:
Tissue inhibitor of metalloproteinase (TIMP-1) at about 16630 (pg/mL) to about 166300 (pg/mL); or
Tissue inhibitor of metalloproteinase (TIMP-2) at about 2960 (pg/mL) to about 29600 (pg/mL); or
Tissue inhibitor of metalloproteinase (TIMP-4) at about 111 (pg/mL) to about 1110 (pg/mL); and
at least one of:
Growth Differentiation Factor (GDF-15) at about 81.25 (pg/mL) to about 812.5 (pg/mL); or
Granulocyte macrophage colony-stimulating factor (GM-CSF) at about 0.21 (pg/mL) to about 2.1 (pg/mL); and
Interferon gamma (IFNγ) at about 2.75 (pg/mL) to about 27.5 (pg/mL); and
at least one of:
Interleukin 1 alpha (IL1-alpha) at about 12.5 (pg/mL) to about 125 (pg/mL); or
Interleukin 1 Beta (IFN-β) at about 27.8 (pg/mL) to about 278 (pg/mL); or
Interleukin 1 receptor antagonist (IL-1ra) at about 78.5 (pg/mL) to about 785 (pg/mL); or
Interleukin 5 (IL-5) at about 2.88 (pg/mL) to about 28.8 (pg/mL); or
Interleukin 7 (IL-7) at about 1.37 (pg/mL) to about 13.7 (pg/mL); or
Interleukin 12 p40 (IL-12p40) at about 9.55 (pg/mL) to about 95.5 (pg/mL); or
Interleukin 12 p70 (IL-12p70) at about 0.79 (pg/mL) to about 7.9 (pg/mL); or
Interleukin 15 (IL-15) at about 1.35 (pg/mL) to about 13.5 (pg/mL); or
Interleukin 17 (IL-17) at about 1.1 (pg/mL) to about 11 (pg/mL); or
Interleukin 16 (IL-16) at about 34.4 (pg/mL) to about 344 (pg/mL); and
Macrophage colony-stimulating factor (MCSF) at about 4.3 (pg/mL) to about 43 (pg/mL); and
Osteoprotegerin (OPG) at about 319.69 (pg/mL) to about 3196.9 (pg/mL); and
B lymphocyte chemoattractant (CXCL13) (BLC) at about 60 (pg/mL) to about 600 (pg/mL); and
Chemokine ligand 1 (CCL1) (1-309) at about 4 (pg/mL) to about 40 (pg/mL); and
Eotaxin-2 at about 0.13 (pg/mL) to about 1.3 (pg/mL); and
Monocyte chemotactic protein 1 (CCL2) (MCP-1) at about 76.95 (pg/mL) to about 769.5 (pg/mL); and
Monokine induced by gamma interferon (CXCL9) (MIG) at about 780 (pg/mL) to about 7800 (pg/mL); and
at least one of:
Macrophage inflammatory protein 1 alpha (CCL3) (MIP-1α) at about 13.08 (pg/mL) to about 130.8 (pg/mL); or
Macrophage inflammatory protein 1 beta (CCL4) (MIP-1β) at about 6.56 (pg/mL) to about 65.6 (pg/mL); or
Macrophage inflammatory protein 1D (MIP-5, CCL15) (MIP-1d) at about 4.3 (pg/mL) to about 43 (pg/mL); and
Regulated on activation, normal T cell expressed and secreted (CCLS) (RANTES) at about 203 (pg/mL) to about 2030 (pg/mL); and
Brain-derived neurotrophic factor (BDNF) at about 45.03 (pg/mL) to about 450.3 (pg/mL); and
Bone morphogenetic protein 5 (BMP-5) at about 90.55 (pg/mL) to about 905.5 (pg/mL); and
Endocrine gland-derived vascular endothelial growth factor (EG-VEGF) at about 496.68 (pg/mL) to about 4966.8 (pg/mL); and
Fibroblast growth factor 4 (FGF-4) at about 353.37 (pg/mL) to about 3533.7 (pg/mL); and
Keratinocyte growth factor (FGF-7) at about 46.7 (pg/mL) to about 467 (pg/mL); and
Growth hormone (GH) at about 114.23 (pg/mL) to about 1142.3 (pg/mL); and
at least one of:
Insulin-like growth factor (IGF-I) at about 27.65 (pg/mL) to about 276.5 (pg/mL); or
Insulin-like growth factor binding protein-1 (IGFBP-1) at about 353.51 (pg/mL) to about 3535.1 (pg/mL); or
Insulin-like growth factor binding protein-2 (IGFBP-2) at about 1072.52 (pg/mL) to about 10725.2 (pg/mL); or
Insulin-like growth factor binding protein-3 (IGFBP-3) at about 7701.19 (pg/mL) to about 77011.9 (pg/mL); or
Insulin-like growth factor binding protein-4 (IGFBP-4) at about 2954.34 (pg/mL) to about 29543.4 (pg/mL); or
Insulin-like growth factor binding protein-6 (IGFBP-6) at about 5162.16 (pg/mL) to about 51621.6 (pg/mL),
thereby regenerating, rejuvenating or remodeling the connective tissue.

6. The method of claim 5, wherein the formulation comprises the following recombinant peptides:
at least one of:
Basic fibroblast growth factor (bFGF) at about 4440 (pg/mL) to about 13320 (pg/mL); or
Epidermal growth factor (EGF) at about 16.4 (pg/mL) to about 49.2 (pg/mL); or
Granulocyte colony-stimulating factor (GCSF) at about 144 (pg/mL) to about 432 (pg/mL); and
at least one of:
Platelet-derived growth factor (PDGF-AA) at about 34327 (pg/mL) to about 102981 (pg/mL); or
Platelet-derived growth factor (PDGF-BB) at about 106 (pg/mL) to about 318 (pg/mL); or
Placental growth factor (PLGF) at about 370 (pg/mL) to about 1110 (pg/mL); and
at least one of:
Transforming growth factor alpha (TGF)-alpha at about 3.4 (pg/mL) to about 10.2 (pg/mL); or
Transforming growth factor beta 1 (TGF-B1) at about 1180 (pg/mL) to about 3540 (pg/mL); and
at least one of:
interleukin 4 (IL-4) at about 2.2 (pg/mL) to about 6.6 (pg/mL); or
interleukin 6 (IL-6) at about 74 (pg/mL) to about 222 (pg/mL); or
interleukin 8 (IL-8) at about 2875 (pg/mL) to about 8625 (pg/mL); or
interleukin 10 (IL-10) at about 4.1 (pg/mL) to about 12.3 (pg/mL); and
at least one of:
Tissue inhibitor of metalloproteinase (TIMP-1) at about 16630 (pg/mL) to about 49890 (pg/mL); or
Tissue inhibitor of metalloproteinase (TIMP-2) at about 2960 (pg/mL) to about 8880 (pg/mL); or
Tissue inhibitor of metalloproteinase (TIMP-4) at about 111 (pg/mL) to about 333 (pg/mL); and
at least one of:
Growth Differentiation Factor (GDF-15) at about 81.25 (pg/mL) to about 243.75 (pg/mL); or
Granulocyte macrophage colony-stimulating factor (GM-CSF) at about 0.21 (pg/mL) to about 0.63 (pg/mL); and
Interferon gamma (IFNγ) at about 2.75 (pg/mL) to about 8.25 (pg/mL); and
at least one of:
Interleukin 1 alpha (IL1-alpha) at about 12.5 (pg/mL) to about 37.5 (pg/mL); or
Interleukin 1 Beta (IFN-β) at about 27.8 (pg/mL) to about 83.4 (pg/mL); or
Interleukin 1 receptor antagonist (IL-1ra) at about 78.5 (pg/mL) to about 235.5 (pg/mL); or
Interleukin 5 (IL-5) at about 2.88 (pg/mL) to about 8.64 (pg/mL); or
Interleukin 7 (IL-7) at about 1.37 (pg/mL) to about 4.11 (pg/mL); or
Interleukin 12 p40 (IL-12p40) at about 9.55 (pg/mL) to about 28.65 (pg/mL); or
Interleukin 12 p70 (IL-12p70) at about 0.79 (pg/mL) to about 2.37 (pg/mL); or
Interleukin 15 (IL-15) at about 1.35 (pg/mL) to about 4.05 (pg/mL); or
Interleukin 17 (IL-17) at about 1.1 (pg/mL) to about 3.3 (pg/mL); or
Interleukin 16 (IL-16) at about 34.4 (pg/mL) to about 103.2 (pg/mL); and
Macrophage colony-stimulating factor (MCSF) at about 4.3 (pg/mL) to about 12.9 (pg/mL); and
Osteoprotegerin (OPG) at about 319.69 (pg/mL) to about 959.07 (pg/mL); and
B lymphocyte chemoattractant (CXCL13) (BLC) at about 60 (pg/mL) to about 180 (pg/mL); and
Chemokine ligand 1 (CCL1) (1-309) at about 4 (pg/mL) to about 12 (pg/mL); and
Eotaxin-2 at about 0.13 (pg/mL) to about 0.39 (pg/mL); and
Monocyte chemotactic protein 1 (CCL2) (MCP-1) at about 76.95 (pg/mL) to about 230.85 (pg/mL); and
Monokine induced by gamma interferon (CXCL9) (MIG) at about 780 (pg/mL) to about 2340 (pg/mL); and
at least one of:
Macrophage inflammatory protein 1 alpha (CCL3) (MIP-1α) at about 13.08 (pg/mL) to about 39.24 (pg/mL); or
Macrophage inflammatory protein 1 beta (CCL4) (MIP-1β) at about 6.56 (pg/mL) to about 19.68 (pg/mL); or
Macrophage inflammatory protein 1D (MIP-5, CCL15) (MIP-1d) at about 4.3 (pg/mL) to about 12.9 (pg/mL); and
Regulated on activation, normal T cell expressed and secreted (CCL5) (RANTES) at about 203 (pg/mL) to about 609 (pg/mL); and
Brain-derived neurotrophic factor (BDNF) at about 45.03 (pg/mL) to about 135.09 (pg/mL); and
Bone morphogenetic protein 5 (BMP-5) at about 90.55 (pg/mL) to about 271.65 (pg/mL); and
Endocrine gland-derived vascular endothelial growth factor (EG-VEGF) at about 496.68 (pg/mL) to about 1490.04 (pg/mL); and
Fibroblast growth factor 4 (FGF-4) at about 353.37 (pg/mL) to about 1060.11 (pg/mL); and
Keratinocyte growth factor (FGF-7) at about 46.7 (pg/mL) to about 140.1 (pg/mL); and
Growth hormone (GH) at about 114.23 (pg/mL) to about 342.69 (pg/mL); and
at least one of:
Insulin-like growth factor (IGF-I) at about 27.65 (pg/mL) to about 82.95 (pg/mL); or
Insulin-like growth factor binding protein-1 (IGFBP-1) at about 353.51 (pg/mL) to about 1060.53 (pg/mL); or
Insulin-like growth factor binding protein-2 (IGFBP-2) at about 1072.52 (pg/mL) to about 3217.56 (pg/mL); or
Insulin-like growth factor binding protein-3 (IGFBP-3) at about 7701.19 (pg/mL) to about 23103.57 (pg/mL); or
Insulin-like growth factor binding protein-4 (IGFBP-4) at about 2954.34 (pg/mL) to about 8863.02 (pg/mL); or
Insulin-like growth factor binding protein-6 (IGFBP-6) at about 5162.16 (pg/mL) to about 15486.48 (pg/mL).

7. The method of claim 6, wherein the formulation comprises the following recombinant peptides:
at least one of:
Basic fibroblast growth factor (bFGF) at about 13320 (pg/mL) to about 44400 (pg/mL); or
Epidermal growth factor (EGF) at about 49.2 (pg/mL) to about 164 (pg/mL); or
Granulocyte colony-stimulating factor (GCSF) at about 432 (pg/mL) to about 1440 (pg/mL); and
at least one of:
Platelet-derived growth factor (PDGF-AA) at about 102981 (pg/mL) to about 343270 (pg/mL); or
Platelet-derived growth factor (PDGF-BB) at about 318 (pg/mL) to about 1060 (pg/mL); or
Placental growth factor (PLGF) at about 1110 (pg/mL) to about 3700 (pg/mL); and
at least one of:
Transforming growth factor alpha (TGF)-alpha at about 10.2 (pg/mL) to about 34 (pg/mL); or
Transforming growth factor beta 1 (TGF-B1) at about 3540 (pg/mL) to about 11800 (pg/mL); and
at least one of:
interleukin 4 (IL-4) at about 6.6 (pg/mL) to about 22 (pg/mL); or
interleukin 6 (IL-6) at about 222 (pg/mL) to about 740 (pg/mL); or
interleukin 8 (IL-8) at about 8625 (pg/mL) to about 28750 (pg/mL); or
interleukin 10 (IL-10) at about 12.3 (pg/mL) to about 41 (pg/mL); and
at least one of:
Tissue inhibitor of metalloproteinase (TIMP-1) at about 49890 (pg/mL) to about 166300 (pg/mL); or
Tissue inhibitor of metalloproteinase (TIMP-2) at about 8880 (pg/mL) to about 29600 (pg/mL); or
Tissue inhibitor of metalloproteinase (TIMP-4) at about 333 (pg/mL) to about 1110 (pg/mL); and
at least one of:
Growth Differentiation Factor (GDF-15) at about 243.75 (pg/mL) to about 812.5 (pg/mL); or
Granulocyte macrophage colony-stimulating factor (GM-CSF) at about 0.63 (pg/mL) to about 2.1 (pg/mL); and
Interferon gamma (IFNγ) at about 8.25 (pg/mL) to about 27.5 (pg/mL); and
at least one of:
Interleukin 1 alpha (IL1-alpha) at about 37.5 (pg/mL) to about 125 (pg/mL); or
Interleukin 1 Beta (IFN-β) at about 83.4 (pg/mL) to about 278 (pg/mL); or
Interleukin 1 receptor antagonist (IL-1ra) at about 235.5 (pg/mL) to about 785 (pg/mL); or
Interleukin 5 (IL-5) at about 8.64 (pg/mL) to about 28.8 (pg/mL); or
Interleukin 7 (IL-7) at about 4.11 (pg/mL) to about 13.7 (pg/mL); or
Interleukin 12 p40 (IL-12p40) at about 28.65 (pg/mL) to about 95.5 (pg/mL); or
Interleukin 12 p70 (IL-12p70) at about 2.37 (pg/mL) to about 7.9 (pg/mL); or
Interleukin 15 (IL-15) at about 4.05 (pg/mL) to about 13.5 (pg/mL); or
Interleukin 17 (IL-17) at about 3.3 (pg/mL) to about 11 (pg/mL); or
Interleukin 16 (IL-16) at about 103.2 (pg/mL) to about 344 (pg/mL); and
Macrophage colony-stimulating factor (MCSF) at about 12.9 (pg/mL) to about 43 (pg/mL); and
Osteoprotegerin (OPG) at about 959.07 (pg/mL) to about 3196.9 (pg/mL); and
B lymphocyte chemoattractant (CXCL13) (BLC) at about 180 (pg/mL) to about 600 (pg/mL); and
Chemokine ligand 1 (CCL1) (1-309) at about 12 (pg/mL) to about 40 (pg/mL); and
Eotaxin-2 at about 0.39 (pg/mL) to about 1.3 (pg/mL); and
Monocyte chemotactic protein 1 (CCL2) (MCP-1) at about 230.85 (pg/mL) to about 769.5 (pg/mL); and
Monokine induced by gamma interferon (CXCL9) (MIG) at about 2340 (pg/mL) to about 7800 (pg/mL); and
at least one of:
Macrophage inflammatory protein 1 alpha (CCL3) (MIP-1α) at about 39.24 (pg/mL) to about 130.8 (pg/mL); or
Macrophage inflammatory protein 1 beta (CCL4) (MIP-1β) at about 19.68 (pg/mL) to about 65.6 (pg/mL); or
Macrophage inflammatory protein 1D (MIP-5, CCL15) (MIP-1d) at about 12.9 (pg/mL) to about 43 (pg/mL); and
Regulated on activation, normal T cell expressed and secreted (CCLS) (RANTES) at about 609 (pg/mL) to about 2030 (pg/mL); and
Brain-derived neurotrophic factor (BDNF) at about 135.09 (pg/mL) to about 450.3 (pg/mL); and
Bone morphogenetic protein 5 (BMP-5) at about 271.65 (pg/mL) to about 905.5 (pg/mL); and
Endocrine gland-derived vascular endothelial growth factor (EG-VEGF) at about 1490.04 (pg/mL) to about 4966.8 (pg/mL); and
Fibroblast growth factor 4 (FGF-4) at about 1060.11 (pg/mL) to about 3533.7 (pg/mL); and
Keratinocyte growth factor (FGF-7) at about 140.1 (pg/mL) to about 467 (pg/mL); and
Growth hormone (GH) at about 342.69 (pg/mL) to about 1142.3 (pg/mL); and
at least one of:
Insulin-like growth factor (IGF-I) at about 82.95 (pg/mL) to about 276.5 (pg/mL); or
Insulin-like growth factor binding protein-1 (IGFBP-1) at about 1060.53 (pg/mL) to about 3535.1 (pg/mL); or
Insulin-like growth factor binding protein-2 (IGFBP-2) at about 3217.56 (pg/mL) to about 10725.2 (pg/mL); or
Insulin-like growth factor binding protein-3 (IGFBP-3) at about 23103.57 (pg/mL) to about 77011.9 (pg/mL); or
Insulin-like growth factor binding protein-4 (IGFBP-4) at about 8863.02 (pg/mL) to about 29543.4 (pg/mL); or
Insulin-like growth factor binding protein-6 (IGFBP-6) at about 15486.48 (pg/mL) to about 51621.6 (pg/mL).

8. A method of treating a connective tissue, comprising:
administering via injection to the connective tissue a formulation, comprising:
a. a carrier; and
b. at least 20 of the following recombinant peptides at the following amounts:

| | |
|---|---|
| Recombinant Peptide | About (pg/mL) to About (pg/mL) |
| | | |
|---|---|---|
| ENA-78 | 0.2 | 1608.3517 |
| GRO | 0.1 | 1700 |
| IL-18 BPa | 0.4 | 4081.5124 |
| IL-31 | 0.1 | 196.84694 |
| I-TAC | 0.4 | 4233.881 |
| LIF | 0.3 | 3378.533 |
| LIGHT | 0.3 | 2500.93 |
| Lymphotactin | 2.4 | 23932.025 |
| MCP-2 | 0.1 | 551.96996 |
| MCP-3 | 0.1 | 609.13575 |
| MCP-4 | 0.1 | 292.47905 |
| MIP-3a | 0.1 | 229.96335 |
| MIP-3b | 0.1 | 3500 |
| MPIF-1 | 0.1 | 399.99117 |
| MSP | 2.1 | 20958.324 |
| NAP-2 | 0.1 | 689.13343 |
| OPN | 0.1 | 756.41221 |
| PARC | 0.1 | 7300 |
| PF4 | 0.1 | 433400 |
| SDF-1a | 0.2 | 1562.9177 |
| TECK | 0.9 | 9440.3426 |
| ADAM9 | 40.0 | 399782.39 |
| Cathepsin L | 5.1 | 50758.58 |
| CD97 | 165.7 | 1657408.3 |
| FAP | 0.9 | 8578.3726 |
| Galectin-3 | 37.3 | 372658.09 |
| IGF-2R | 598.5 | 5984898.6 |
| Neprilysin | 83.2 | 832141.8 |
| NOV | 0.2 | 2458.9006 |
| PGRP-S | 0.9 | 9359.735 |
| Serpin A4 | 464.1 | 4640831.7 |
| TLR2 | 12.2 | 122039.83 |
| Transferrin | 462.6 | 16238900 |
| Albumin | 23.9 | 540900 |
| CA19-9 | 151.1 | 1511427.3 |
| CD163 | 0.8 | 8373.8074 |
| Cystatin C | 8.1 | 80573.142 |
| Decorin | 88.7 | 887080.79 |
| Dkk-3 | 4.3 | 43053.353 |
| Fetuin A | 22.6 | 199374000 |
| FOLR1 | 5.8 | 57601.162 |
| HAI-2 | 0.8 | 7764.0163 |
| IL-27 | 0.1 | 1411.1913 |
| LAG-3 | 2.1 | 20627.085 |
| RBP4 | 165.5 | 5745200 |
| TACI | 14.1 | 141318.11 |
| Adiponectin | 24.5 | 244589.78 |
| ANGPTL4 | 3.3 | 32962.016 |
| B2M | 6.9 | 520100 |
| BCAM | 1.9 | 19230.662 |
| CA125 | 10.9 | 138194000 |
| CEA | 10.2 | 102497.22 |
| CRP | 1.1 | 10624.726 |
| Ferritin | 63.6 | 635916.3 |
| FSH | 0.7 | 7136.6774 |
| hCGb | 2.3 | 22543.625 |
| IL-3 | 1.6 | 15602.928 |
| IL-21 | 32.5 | 324558.31 |
| Leptin | 1.0 | 9811.1399 |
| MMP-1 | 3.8 | 38265.885 |
| MMP-2 | 49.3 | 493442.5 |
| MMP-3 | 1.7 | 16884.21 |
| MMP-8 | 1.5 | 18900 |
| MMP-9 | 3.3 | 33175.025 |
| MMP-10 | 0.1 | 1300 |
| MMP-13 | 1.0 | 59900 |
| NCAM-1 | 7.5 | 74728.399 |
| Nidogen-1 | 1.1 | 10663700 |
| NSE | 1.6 | 16204.701 |
| OSM | 2.0 | 19589.185 |
| Prolactin | 44.4 | 444334.55 |
| PSA-free | 1.7 | 16933.554 |
| Siglec-9 | 1.9 | 18778.24 |
| TACE | 1.8 | 174800 |
| TIMP-4 | 0.6 | 5910.0444 |
| Activin A | 36.7 | 366604.67 |
| AgRP | 0.1 | 1235.4788 |
| Angiogenin | 0.5 | 64400 |
| ANG-1 | 1.0 | 10092.204 |
| Angiostatin | 41.3 | 5376000 |
| Cathepsin S | 1.0 | 10400 |
| CD40 | 1.5 | 14967.535 |
| Cripto-1 | 0.5 | 4975.1248 |
| DAN | 10.5 | 105061.11 |
| DKK-1 | 2.7 | 27128.196 |
| E-Cadherin | 14.2 | 141675.42 |
| EpCAM | 0.3 | 3198.2786 |
| FAS L | 3.1 | 30735.803 |
| Fcg RIIBC | 8.1 | 81141.464 |
| Follistatin | 1.7 | 12500 |
| Galectin-7 | 3.9 | 292400 |
| ICAM-2 | 14.9 | 149308.12 |
| IL-13 R1 | 0.1 | 6300 |
| IL-13 R2 | 4.7 | 209000 |
| IL-17B | 0.5 | 5206.2476 |
| IL-2 Ra | 0.6 | 6040.3327 |
| IL-23 | 0.3 | 34800 |
| LAP(TGFb1) | 0.1 | 52700 |
| NrCAM | 3.1 | 31375.447 |
| PAI-1 | 0.1 | 2103300 |
| PDGF-AB | 1.3 | 12601.852 |
| Resistin | 3.0 | 29595.005 |
| SDF-1b | 0.4 | 3688.2262 |
| gp130 | 15.7 | 157247.2 |
| Shh-N | 2.4 | 23906.235 |
| Siglec-5 | 2.4 | 23926.46 |
| IL-1 R4 | 0.7 | 7107.8063 |
| TGFb2 | 2.8 | 27547.662 |
| Tie-2 | 7.7 | 76819.804 |
| TPO | 6.7 | 67405.385 |
| TRAIL R4 | 5.8 | 57815.572 |
| TREM-1 | 2.4 | 23962.94 |
| VEGF-C | 1.2 | 11670.245 |
| VEGF R1 | 5.9 | 444700 |
| ADAM8 | 0.6 | 5937.2801 |
| B7-H3 | 7.1 | 107400 |
| Cadherin-13 | 1.1 | 10951.828 |
| CD48 | 23.3 | 233338.03 |
| CD99 | 0.3 | 83700 |
| CD229 | 4.2 | 42331.572 |
| CEACAM-5 | 20.7 | 366700 |
| Cystatin A | 0.1 | 2500 |
| Cystatin B | 0.6 | 77300 |
| Cystatin E M | 0.5 | 247900 |
| Desmoglein 2 | 0.1 | 12800 |
| DR3 | 21.2 | 212172.13 |
| ErbB4 | 0.1 | 1070.307 |
| ESAM | 0.3 | 2735.6701 |
| FGF-21 | 0.7 | 6824.9236 |
| Galectin-2 | 11.9 | 119140.34 |
| Galectin-9 | 1.9 | 367600 |
| JAM-B | 2.2 | 22105.271 |
| Kallikrein 5 | 12.9 | 129388.03 |
| Midkine | 2.0 | 32000 |
| Pentraxin 3 | 3.1 | 100900 |
| Pref-1 | 55.5 | 554824.44 |
| Siglec-10 | 26.7 | 266781.06 |
| SLAM | 13.8 | 138165.88 |
| SP-D | 11.0 | 109685.54 |
| Syndecan-4 | 0.3 | 77900 |
| Testican 2 | 3.0 | 30239.317 |
| TIM-3 | 2.5 | 25444.14 |
| TLR4 | 1.2 | 11729.567 |
| TRAIL | 0.4 | 4190.4283 |
| ULBP-1 | 5.4 | 54340.852 |
| ADAMTS13 | 3.9 | 38692.371 |
| Aggrecan | 0.0 | 421.92648 |
| Angiotensinogen | 3.3 | 368600 |
| B7-H1 | 7.0 | 69781.9 |
| BMPR-IA | 9.2 | 92023.533 |
| BMPR-II | 10.9 | 109051.17 |
| Cadherin-11 | 0.4 | 4500 |
| CD27 | 0.4 | 4013.197 |
| Ck beta 8-1 | 0.2 | 1524.2229 |
| EMMPRIN | 2.1 | 1796500 |
| Follistatin-like 1 | 6.1 | 61435.008 |
| Fractalkine | 1.8 | 18345.526 |
| Galectin-1 | 5.0 | 1499500 |
| GITR L | 6.3 | 63489.982 |
| Granulysin | 3.7 | 15016500 |
| IL-1 R3 | 0.1 | 47300 |
| IL-15 R | 0.2 | 2281.6196 |
| IL-17E | 0.1 | 1424.0357 |
| L1CAM-2 | 28.0 | 280303.55 |
| LRIG3 | 4.6 | 46162.447 |
| MEPE | 0.3 | 2501.2571 |
| Nectin-4 | 2.0 | 19745.357 |
| Periostin | 0.1 | 11094900 |
| Persephin | 6.6 | 65802.305 |
| Renin | 0.2 | 2400 |
| RGM-B | 1.9 | 19158.505 |
| ROBO3 | 0.9 | 9057.6684 |
| S100A8 | 0.5 | 4948.3702 |
| Siglec-7 | 0.4 | 4398.0532 |
| Syndecan-3 | 11.0 | 110129.64 |
| Thrombospondin-2 | 2.5 | 24586.146 |
| Thrombospondin-5 | 0.1 | 616.61911 |
| Tie-1 | 2.6 | 26428.685 |
| ULBP-2 | 6.6 | 66122.681 |
| ANGPTL3 | 6.0 | 59846.266 |
| bIG-H3 | 12.0 | 4221200 |
| CA9 | 1.2 | 12034.621 |
| Cathepsin B | 2.3 | 23154.156 |
| CD23 | 0.7 | 6539.8579 |
| CHI3L1 | 2.1 | 21118.734 |
| Dkk-4 | 26.5 | 264677.25 |
| DPPIV | 48.6 | 35659000 |
| EDA-A2 | 14.4 | 144004.74 |
| Epo R | 3.3 | 33108.103 |
| FGF-6 | 1.7 | 17005.984 |
| FGF-9 | 2.7 | 26713.438 |
| Gas 1 | 1.2 | 11557.807 |
| IGFBP-5 | 124.6 | 1245948 |
| IL-1 F5 | 0.6 | 6349.2077 |
| IL-1 F6 | 10.3 | 102653.98 |
| IL-1 F8 | 0.2 | 1561.463 |
| IL-1 F9 | 26.8 | 268132.23 |
| IL-1 F10 | 70.6 | 706256.28 |
| IL-1 R5 | 1.6 | 15748.91 |
| IL-17C | 1.6 | 15815.29 |
| IL-18 | 1.2 | 12203.999 |
| IL-20 | 5.1 | 1194000 |
| IL-34 | 0.4 | 3547.4731 |
| IL-5 Ra | 104.0 | 1040223.9 |
| IL-10 Ra | 506.6 | 5066467.2 |
| Layilin | 1.5 | 14863.552 |
| Leptin R | 69.3 | 693409.21 |
| Marapsin | 2.6 | 25733.96 |
| Mer | 19.1 | 191389.77 |
| MMP-7 | 2.2 | 21509.624 |
| P-Cadherin | 1.3 | 12621.709 |
| Prostasin | 3.3 | 32547.02 |
| PSMA | 263.0 | 2629597.7 |
| SIGIRR | 40.9 | 409390.78 |
| TGFb RIII | 36.2 | 362176 |
| TF | 0.6 | 88400 |
| Albumin | 23.9 | 540900 |
| CA19-9 | 151.1 | 1511427.3 |
| CD163 | 0.8 | 8373.8074 |
| Cystatin C | 8.1 | 80573.142 |
| Decorin | 88.7 | 887080.79 |
| Dkk-3 | 4.3 | 43053.353 |
| Fetuin A | 22.6 | 199374200 |
| FOLR1 | 5.8 | 57601.162 |
| HAI-2 | 0.8 | 7764.0163 |
| IL-27 | 0.1 | 1411.1913 |
| LAG-3 | 2.1 | 20627.085 |
| RBP4 | 16.5 | 5745200 |
| TACI | 14.1 | 141318.11 |
| AR | 2.8 | 27899.188 |
| BDNF | 0.1 | 480.40447 |
| bFGF | 2.0 | 25200 |
| BMP-4 | 0.2 | 1748.5354 |
| BMP-5 | 200.8 | 2007757.6 |
| BMP-7 | 23.3 | 233472.15 |
| b-NGF | 0.1 | 50.686799 |
| EGF | 0.1 | 500 |
| EGF R | 12.5 | 187600 |
| FGF-4 | 8.5 | 84904.202 |
| GDF-15 | 0.1 | 19700 |
| GH | 0.4 | 3732.1841 |
| HB-EGF | 0.2 | 2360.5215 |
| HGF | 2.6 | 81800 |
| IGFBP-1 | 0.1 | 1781200 |
| IGFBP-2 | 2.5 | 91300 |
| IGFBP-3 | 53.3 | 532982.09 |
| IGFBP-4 | 186.2 | 1861630.9 |
| IGFBP-6 | 4.5 | 183100 |
| Insulin | 1,032.6 | 10326254 |
| MCSF R | 39.3 | 393031.19 |
| NGF R | 0.1 | 1138.7885 |
| NT-3 | 0.2 | 1683.605 |
| PDGF-AA | 0.5 | 4770.0148 |
| PIGF | 0.1 | 10600 |
| SCF | 0.1 | 841.7876 |
| SCF R | 10.8 | 108461.9 |
| TGFa | 2.8 | 28394.755 |
| TGFb1 | 28.1 | 3166100 |
| TGFb3 | 6.2 | 61590.355 |
| VEGF | 0.2 | 109000 |
| VEGF R2 | 2.4 | 24401.273 |
| BCMA | 26.1 | 261322.53 |
| CEACAM-1 | 8.9 | 88745.144 |
| E-Selectin | 1.8 | 17903.141 |
| Fas | 0.6 | 6328.2205 |
| Flt-3L | 0.2 | 1690.4949 |
| HVEM | 0.8 | 7586.0775 |
| IL-2 Rg | 2.4 | 24170.021 |
| IL-10 Rb | 0.1 | 507.00864 |
| IL-17R | 0.2 | 2040.8122 |
| IL-21R | 0.6 | 5513.407 |
| LIMPII | 69.0 | 689881.16 |
| Lipocalin-2 | 2.8 | 390300 |
| LYVE-1 | 18.5 | 185333.66 |
| NRG1-b1 | 0.1 | 643.53448 |
| PECAM-1 | 0.6 | 6049.5214 |
| TRAIL R3 | 0.2 | 1773.4435 |
| Trappin-2 | 0.1 | 28900 |
| uPAR | 0.6 | 191000 |
| XEDAR | 0.2 | 2036.9134 |

9. The method of claim 8, wherein the formulation comprises about 50 to about 200 of the recombinant peptides.

10. The method of claim 8, wherein the formulation comprises at least about 100 of the recombinant peptides.

11. The method of claim 8, wherein the formulation comprises at least about 20 of the recombinant peptides at the amounts listed below:

| Recombinant Peptide | About (pg/mL) |
|---|---|
| ENA-78 | 1.6 |
| GRO | 0.6-1.7 |
| IL-18 BPa | 4.1 |
| IL-31 | 0.2 |
| I-TAC | 4.2 |
| LIF | 3.4 |
| LIGHT | 2.5 |
| Lymphotactin | 23.9 |
| MCP-2 | 0.6 |
| MCP-3 | 0.6 |
| MCP-4 | 0.3 |
| MIP-3a | 0.2 |
| MIP-3b | 1.1-3.5 |
| MPIF-1 | 0.4 |
| MSP | 21.0 |
| NAP-2 | 0.7 |
| OPN | 0.8 |
| PARC | 0.6-7.3 |
| PF4 | 0.2-433.4 |
| SDF-1a | 1.6 |
| TECK | 9.4 |
| ADAM9 | 399.8 |
| Cathepsin L | 50.8 |
| CD97 | 1,657.4 |
| FAP | 8.6 |
| Galectin-3 | 372.7 |
| IGF-2R | 5,984.9 |
| Neprilysin | 832.1 |
| NOV | 2.5 |
| PGRP-S | 9.4 |
| Serpin A4 | 4,640.8 |
| TLR2 | 122.0 |
| Transferrin | 4625.7-16238.9 |
| Albumin | 239-540.9 |
| CA19-9 | 1,511.4 |
| CD163 | 8.4 |
| Cystatin C | 80.6 |
| Decorin | 887.1 |
| Dkk-3 | 43.1 |
| Fetuin A | 225.6-199374 |
| FOLR1 | 57.6 |
| HAI-2 | 7.8 |
| IL-27 | 1.4 |
| LAG-3 | 20.6 |
| RBP4 | 1654.5-5745.2 |
| TACI | 141.3 |
| Adiponectin | 244.6 |
| ANGPTL4 | 33.0 |
| B2M | 68.6-520.1 |
| BCAM | 19.2 |
| CA125 | 108.8-138194.8 |
| CEA | 102.5 |
| CRP | 10.6 |
| Ferritin | 635.9 |
| FSH | 7.1 |
| hCGb | 22.5 |
| IL-3 | 15.6 |
| IL-21 | 324.6 |
| Leptin | 9.8 |
| MMP-1 | 38.3 |
| MMP-2 | 493.4 |
| MMP-3 | 16.9 |
| MMP-8 | 14.7-18.9 |
| MMP-9 | 33.2 |
| MMP-10 | 0.5-1.3 |
| MMP-13 | 9.9-59.9 |
| NCAM-1 | 74.7 |
| Nidogen-1 | 10.5-10663.7 |
| NSE | 16.2 |
| OSM | 19.6 |
| Prolactin | 444.3 |
| PSA-free | 16.9 |
| Siglec-9 | 18.8 |
| TACE | 17.7-174.8 |
| TIMP-4 | 5.9 |
| Activin A | 366.6 |
| AgRP | 1.2 |
| Angiogenin | 4.6-64.4 |
| ANG-1 | 10.1 |
| Angiostatin | 413.3-5376.2 |
| Cathepsin S | 9.6-10.4 |
| CD40 | 15.0 |
| Cripto-1 | 5.0 |
| DAN | 105.1 |
| DKK-1 | 27.1 |
| E-Cadherin | 141.7 |
| EpCAM | 3.2 |
| FAS L | 30.7 |
| Fcg RIIBC | 81.1 |
| Follistatin | 1.8-12.5 |
| Galectin-7 | 38.8-292.4 |
| ICAM-2 | 149.3 |
| IL-13 R1 | 1.4-6.3 |
| IL-13 R2 | 47.4-209.0 |
| IL-17B | 5.2 |
| IL-2 Ra | 6.0 |
| IL-23 | 2.7-34.8 |
| LAP(TGFb1) | 0.1-52.7 |
| NrCAM | 31.4 |
| PAI-1 | 0.6-2103.3 |
| PDGF-AB | 12.6 |
| Resistin | 29.6 |
| SDF-1b | 3.7 |
| gp130 | 157.2 |
| Shh-N | 23.9 |
| Siglec-5 | 23.9 |
| IL-1 R4 | 7.1 |
| TGFb2 | 27.5 |
| Tie-2 | 76.8 |
| TPO | 67.4 |
| TRAIL R4 | 57.8 |
| TREM-1 | 24.0 |
| VEGF-C | 11.7 |
| VEGF R1 | 59.4-444.7 |
| ADAM8 | 5.9 |
| B7-H3 | 70.9-107.4 |
| Cadherin-13 | 11.0 |
| CD48 | 233.3 |
| CD99 | 3.3-83.7 |
| CD229 | 42.3 |
| CEACAM-5 | 207.0-366.7 |
| Cystatin A | 1.4-2.5 |
| Cystatin B | 5.8-77.3 |
| Cystatin E M | 2.6-247.9 |
| Desmoglein 2 | 0.5-12.8 |
| DR3 | 212.2 |
| ErbB4 | 1.1 |
| ESAM | 2.7 |
| FGF-21 | 6.8 |
| Galectin-2 | 119.1 |
| Galectin-9 | 18.8-367.6 |
| JAM-B | 22.1 |
| Kallikrein 5 | 129.4 |
| Midkine | 19.8-32 |
| Pentraxin 3 | 31.4-100.9 |
| Pref-1 | 554.8 |
| Siglec-10 | 266.8 |
| SLAM | 138.2 |
| SP-D | 109.7 |
| Syndecan-4 | 3.4-77.9 |
| Testican 2 | 30.2 |
| TIM-3 | 25.4 |
| TLR4 | 11.7 |
| TRAIL | 4.2 |
| ULBP-1 | 54.3 |
| ADAMTS13 | 38.7 |
| Aggrecan | 0.4 |
| Angiotensinogen | 32.7-368.6 |
| B7-H1 | 69.8 |
| BMPR-IA | 92.0 |
| BMPR-II | 109.1 |
| Cadherin-11 | 3.6-4.5 |
| CD27 | 4.0 |
| Ck beta 8-1 | 1.5 |
| EMMPRIN | 21.0-1796.5 |
| Follistatin-like 1 | 61.4 |
| Fractalkine | 18.3 |
| Galectin-1 | 49.6-1499.5 |
| GITR L | 63.5 |
| Granulysin | 37.4-15016.5 |
| IL-1 R3 | 0.5-47.3 |
| IL-15 R | 2.3 |
| IL-17E | 1.4 |
| L1CAM-2 | 280.3 |
| LRIG3 | 46.2 |
| MEPE | 2.5 |
| Nectin-4 | 19.7 |
| Periostin | 0.9-11094.9 |
| Persephin | 65.8 |
| Renin | 2.0-2.4 |
| RGM-B | 19.2 |
| ROBO3 | 9.1 |
| S100A8 | 4.9 |
| Siglec-7 | 4.4 |
| Syndecan-3 | 110.1 |
| Thrombospondin-2 | 24.6 |
| Thrombospondin-5 | 0.6 |
| Tie-1 | 26.4 |
| ULBP-2 | 66.1 |
| ANGPTL3 | 59.8 |
| bIG-H3 | 119.6-4221.2 |
| CA9 | 12.0 |
| Cathepsin B | 23.2 |
| CD23 | 6.5 |
| CHI3L1 | 21.1 |
| Dkk-4 | 264.7 |
| DPPIV | 486.4-35659.0 |
| EDA-A2 | 144.0 |
| Epo R | 33.1 |
| FGF-6 | 17.0 |
| FGF-9 | 26.7 |
| Gas 1 | 11.6 |
| IGFBP-5 | 1,245.9 |
| IL-1 F5 | 6.3 |
| IL-1 F6 | 102.7 |
| IL-1 F8 | 1.6 |
| IL-1 F9 | 268.1 |
| IL-1 F10 | 706.3 |
| IL-1 R5 | 15.7 |
| IL-17C | 15.8 |
| IL-18 | 12.2 |
| IL-20 | 51-1194.5 |
| IL-34 | 3.5 |
| IL-5 Ra | 1,040.2 |
| IL-10 Ra | 5,066.5 |
| Layilin | 14.9 |
| Leptin R | 693.4 |
| Marapsin | 25.7 |
| Mer | 191.4 |
| MMP-7 | 21.5 |
| P-Cadherin | 12.6 |
| Prostasin | 32.5 |
| PSMA | 2,629.6 |
| SIGIRR | 409.4 |
| TGFb RIII | 362.2 |
| TF | 5.9-88.4 |
| Albumin | 239.0-540.9 |
| CA19-9 | 1,511.4 |
| CD163 | 8.4 |
| Cystatin C | 80.6 |
| Decorin | 887.1 |
| Dkk-3 | 43.1 |
| Fetuin A | 225.6-199374.2 |
| FOLR1 | 57.6 |
| HAI-2 | 7.8 |
| IL-27 | 1.4 |
| LAG-3 | 20.6 |
| RBP4 | 164.5-5745.2 |
| TACI | 141.3 |
| AR | 27.9 |
| BDNF | 0.5 |
| bFGF | 20.1-25.2 |
| BMP-4 | 1.7 |
| BMP-5 | 2,007.8 |
| BMP-7 | 233.5 |
| b-NGF | 0.1 |
| EGF | 0.1-0.5 |
| EGF R | 125.3-187.6 |
| FGF-4 | 84.9 |
| GDF-15 | 0.4-19.7 |
| GH | 3.7 |
| HB-EGF | 2.4 |
| HGF | 26.0-81.8 |
| IGFBP-1 | 1.4-1781.2 |
| IGFBP-2 | 25.3-91.3 |
| IGFBP-3 | 533.0 |
| IGFBP-4 | 1,861.6 |
| IGFBP-6 | 45.1-183.1 |
| Insulin | 10,326.3 |
| MCSF R | 393.0 |
| NGF R | 1.1 |
| NT-3 | 1.7 |
| PDGF-AA | 4.8 |
| PIGF | 0.8-10.6 |
| SCF | 0.8 |
| SCF R | 108.5 |
| TGFa | 28.4 |
| TGFb1 | 281.2-3166.1 |
| TGFb3 | 61.6 |
| VEGF | 2.0-109.0 |
| VEGF R2 | 24.4 |
| BCMA | 261.3 |
| CEACAM-1 | 88.7 |
| E-Selectin | 17.9 |
| Fas | 6.3 |
| Flt-3L | 1.7 |
| HVEM | 7.6 |
| IL-2 Rg | 24.2 |
| IL-10 Rb | 0.5 |
| IL-17R | 2.0 |
| IL-21R | 5.5 |
| LIMPII | 689.9 |
| Lipocalin-2 | 27.7-390.9 |
| LYVE-1 | 185.3 |
| NRG1-b1 | 0.6 |
| PECAM-1 | 6.0 |
| TRAIL R3 | 1.8 |
| Trappin-2 | 1.0-28.9 |
| uPAR | 6.2-191.0 |
| XEDAR | 2.0 |

12. The method of claim 1, wherein the carrier is a lyophilized or dried carrier.

13. The method of claim 5, wherein the carrier is a lyophilized or dried carrier.

14. The method of claim 8, wherein the carrier is a lyophilized or dried carrier.

15. The method of claim 1, wherein the connective tissue is scar tissue that is remodeled.

16. The method of claim 5, wherein the connective tissue is scar tissue that is remodeled.

17. The method of claim 8, wherein the connective tissue is scar tissue that is remodeled.

18. The method of claim 1, wherein the method provides wrinkle treatment and elimination.

19. The method of claim 5, wherein the method provides wrinkle treatment and elimination.

20. The method of claim 8, wherein the method provides wrinkle treatment and elimination.
